(19) United States
(12) Patent Application Publication
(10) Pub. No.: US 2005/0282190 A1
(43) Pub. Date: Dec. 22, 2005

(54) Modular design and construction of nucleic acid molecules, aptamer-derived nucleic acid constructs, RNA scaffolds, their expression, and methods of use

(76) Inventors: Hua Shi, Ithaca, NY (US); John T. Lis, Ithaca, NY (US)

Correspondence Address: Michael L. Goldman, Esq.;Nixon Peabody LLP, Clinton Square, P.O. Box 31051, Rochester, NY, 14603-1051, US

(21) Appl. No.: 11/103,122

(22) Filed: Apr. 11, 2005

Related U.S. Application Data
(60) Provisional application No. 60/560,895, filed on Apr. 9, 2004.

(51) Int. Cl. C12Q 1/68; C07H 21/04

(52) U.S. Cl. 435/6; 536/24.3

(57) ABSTRACT

The present invention relates to a nucleic acid molecule comprised of first and second nucleic acid elements that each bind a target molecule, and a three-way junction operably linking the first and second nucleic elements. Also disclosed is an RNA scaffold comprising first and second RNA receptor regions operably linked by a three-way junction, wherein the first and second RNA receptor regions each comprise a stem defined by at least two sets of consecutive, canonic, paired bases. A method of using a multivalent nucleic acid aptamer to bring a first and second target molecule into proximity of one another is also disclosed. Also disclosed are constructed DNA molecules, engineered genes, transgenic non-human organisms, methods of modifying activity of target molecules, and functional RNA molecules comprising an RNA scaffold and one or more functional modules. A method for modular design and construction of nucleic acid molecules is also disclosed.

This application claims the priority benefit of U.S. Provisional Patent Application Ser. No. 60/560,895, filed Apr. 9, 2004, which is hereby incorporated by reference in its entirety.

This invention was made in part with government support under U.S. Public Health Service Grant GM40918. The U.S. government may have certain rights to this invention.

### FIELD OF THE INVENTION

This invention relates to aptamer-derived nucleic acid constructs, RNA scaffolds, their expression, and methods of use, as well as modular design and construction of nucleic acid molecules.

### BACKGROUND OF THE INVENTION

The probability of an effective collision between two molecules decreases as a cube function of distance. As a result, a qualitative biological response may be mediated by mere proximity. Inside cells, mechanisms bringing two or more protein molecules together play an important role in cellular regulatory networks (Jeong et al., “Lethality and Centrality in Protein Networks,” Nature 411:41-42 (2001)). The same principle can be employed for therapeutic or experimental purposes. For the purpose of studying and controlling cellular regulatory networks, it is desirable to have the capability of bridging any pair or bringing together more than two molecules or supra-molecular assemblies, especially proteins. A general approach to develop such bridging constructs is to develop two simultaneously available capabilities: the capability of generating ligands to the target proteins or non-proteins at will, and the capability of connecting and recombining these single-site ligands in a single molecular entity at will.

In nature, induced proximity is usually mediated by proteins. There is well-documented involvement of linkers or adapters in signal transduction pathways (Austin et al., “Proximity Versus Allostery: The Role of Regulated Protein Dimerization in Biology,” Chem. Biol. 1:131-136 (1994)). Many proteins are composed of multiple structural domains, each bearing its own interface with its partner or partners. New connections can be generated through domain shuffling (Park et al., “Rewiring MAP Kinase Pathways Using Alternative Scaffold Assembly Mechanisms,” Science 299:1061-1064 (2003)). For example, many transcription factors have a DNA binding domain and an activation domain that fold independently and can be separated and recombined (Sadowski et al., “GAL4-VP 16 is an Unusually Potent Transcriptional Activator,” Nature 335:563-564 (1988)). In some cases, the DNA binding specificity of a DNA binding domain is determined by modular assembly of smaller units such as zinc fingers, which can be used to generate novel DNA recognition specificity (Park et al., “Phenotypic Alteration of Eukaryotic Cells Using Randomized Libraries of Artificial Transcription Factors,” Nat. Biotechnol. 21:1208-1214 (2003)). However, protein constructs have limitations when considered in light of the two capabilities stated above. First, the existing repertoire of useful naturally-occurring single-site domains is limited, and generating novel affinity by de novo protein design is difficult. Second, although novel affinity reagents can be generated in the form of peptide aptamers, the less controllable protein folding process makes it difficult to connect them together without compromising their individual activities.

A few small organic molecules have been found to be able to function as inducers of protein dimerization. Some of these, such as FK506 and cyclosporin A, have been further developed and used in experiments to control intracellular signaling (Crabtree et al., “Three-Part Inventions: Intracellular Signaling and Induced Proximity,” Trends Biochem. Sci. 21:418-422 (1996)). Novel ligands to proteins in the form of small compounds are being generated efficiently through combinatorial chemistry and other methods (Freidinger, “Nonpeptidic Ligands for Peptide and Protein Receptors,” Curr. Opin. Chem. Biol. 3:395-406 (1999)). While small molecules possess the first capability, their second capability is severely limited by their size. Protein surfaces in direct contact with each other usually involve an area of about 1600 Å² with relatively flat topography (Lo Conte et al., “The Atomic Structure of Protein-Protein Recognition Sites,” J. Mol. Biol. 285:2177-2198 (1999)), but small molecules of less than 500 Da have less than 500 Å² of total solvent-accessible surface area (Gadek et al., “Small Molecule Antagonists of Proteins,” Biochem. Pharmacol. 65:1-8 (2003)). With this restriction, only very few inducers of dimerization can be constructed, and it is extremely difficult, if not impossible, to bring more than two proteins together using a small molecule.

While both goals (generating ligands to the target proteins or non-proteins at will, and connecting and recombining these single-site ligands in a single molecular entity at will) are difficult to meet simultaneously by proteins or small compounds, an alternative approach seems to be better fit for the task of inducing proximity. The methodology of in vitro selection (Ellington et al., “In Vitro Selection of RNA and DNA Molecules that Bind Specific Ligands,” Nature 346:818-822 (1990); Tuerk et al., “Systematic Evolution of Ligands by Exponential Enrichment: RNA Ligands to Bacteriophage T4 DNA Polymerase,” Science 249:505-510 (1990)) can produce ligands (aptamers) against targets of diverse chemical identities, including ions, lipids, antibiotics, metabolites, proteins, organelles, and viral particles, thus fulfilling the requirements of the first capability as efficiently as small molecules (Gold et al., “Diversity of Oligonucleotide Functions,” Annu. Rev. Biochem. 64:763-797 (1995)). Unlike protein, most of RNA folding energy can be attributed to its secondary structures (Flamm et al., “RNA Folding at Elementary Step Resolution,” RNA 6:325-338 (2000)), making it easier to piece together different elements in a composite construct, required by the second capability stated above. A case of naturally occurring RNA induced protein proximity has been reported recently. In mammalian cells, 7SK snRNA brings the HEXIM1 and P-TEFb proteins together, resulting in the inhibition of P-TEFb Kinase by HEXIM1 (Yik et al., “Inhibition of P-TEFb (CDK9/Cyclin T) Kinase and RNA Polymerase II Transcription by the Coordinated Actions of HEXIM1 and 7SK snRNA,” Mol. Cell 12:971-982 (2003)). While 7SK snRNA seems to bind to both proteins, its structure and binding sites are not well characterized. Artificial constructs using RNA to mediate protein proximity have been developed in a three-hybrid system in yeast to detect RNA-protein interactions (U.S. Pat. Nos. 5,610,015, 5,677,131, and 5,750,667 to Wickens et al.). This is a screening or selection technique in which the structural relationship of the two components in the RNA “hybrid” was not designed. This “hybrid” would only work when both components, the MS2 coat protein ligand RNA and an RNA fragment undefined and to be tested, function independently in a single RNA transcript. Multivalent aptamer constructs in which two or more aptamers are strung together in one molecule by design have previously been described (U.S. Pat. No. 6,458,559 to Shi et al.). This method can be extended to aptamers or non-aptamer functional elements with different specificity. For in vivo use, RNA constructs have the added advantage of being less antigenic than peptides.

Most cellular functions are actualized by multi-protein complexes and regulated by multi-protein networks. RNA constructs have been made to imitate one aspect of proteins: bio-synthesis under genetic control (U.S. Pat. No. 6,458,559 to Shi et al. and U.S. patent application Ser. No. 10/173,368 to Shi et al.).

The present invention is directed to overcoming the above-identified deficiencies in the art and achieving the above-identified objectives.

### SUMMARY OF THE INVENTION

A first aspect of the present invention relates to a nucleic acid molecule that includes first and second nucleic acid elements that each bind a target molecule, and a three-way junction. The three-way junction is formed of the same type of nucleic acid as the first and second nucleic acid elements, and operably links the first and second nucleic acid elements. The nucleic acid molecule can be either DNA or RNA, or derivatives thereof. This aspect of the invention further relates to constructed DNA molecules and engineered genes that encode the RNA molecules of the present invention, as well as expression systems and recombinant host cells that contain the DNA molecules or engineered genes. Methods of expressing the RNA molecules of the present invention are also disclosed.

A second aspect of the present invention relates to a transgenic non-human organism whose somatic and/or germ cells contain an engineered gene of the present invention. The RNA molecule encoded by the engineered gene can be used to modify the activity of a target molecule, such as a protein, and thereby cause a phenotypic change (as compared to an otherwise identical non-transgenic organism).

A third aspect of the present invention relates to a method of modifying the activity of a target molecule in a cell. This method can be carried out by introducing into a cell a nucleic acid molecule of the present invention, where the nucleic acid molecule has an affinity for the target molecule that is sufficient to bind to the target molecule and thereby modify the activity thereof. The cell can be located either in vivo or ex vivo. Alternatively, this method can be practiced in an extracellular environment, e.g., in vitro.

A fourth aspect of the present invention relates to an RNA scaffold that includes first and second RNA receptor regions operably linked by a three-way junction. The first and second RNA receptor regions each contain a stem defined by at least two sets of consecutive, canonic paired bases. The receptor regions are adapted for receiving a functional RNA module that is characterized by a desired or selected activity. This aspect of the present invention further relates to constructed DNA molecules and engineered genes that encode the RNA scaffolds of the present invention, as well as expression systems and recombinant host cells that contain the constructed DNA molecules or engineered genes. Methods of expressing the RNA scaffolds of the present invention are also disclosed.

A fifth aspect of the present invention relates to a method of using a multivalent nucleic acid aptamer to bring a first and second target molecule into proximity of one another. This method involves providing a multivalent aptamer comprising first, second, and third aptamer sequences, wherein the first and second aptamer sequences are operably linked by a three-way junction, and the third aptamer sequence is operably linked to the first and second aptamer sequences, and wherein each of the first and second aptamer sequences is capable of binding the first target molecule, and the third aptamer sequence is capable of binding the second target molecule, which is different from the first target molecule. The method further involves exposing the multivalent nucleic acid aptamer to one or more samples that may contain the first and second target molecules, and then determining whether the multivalent nucleic acid aptamer binds the first and second target molecules.

A sixth aspect of the present invention relates to a method of detecting the presence of a target molecule in a sample. This method involves exposing a nucleic acid molecule of the present invention to a sample under conditions effective to allow binding of a target molecule in the sample by the nucleic acid molecule and then determining whether the nucleic acid molecule has bound to the target molecule.

A seventh aspect of the present invention relates to a method for modular design and construction of nucleic acid molecules. This method involves providing one or more structural nucleic acid modules and one or more functional nucleic acid modules. At least one of each of the structural and functional nucleic acid modules is combinatorially joined to form a single molecular entity according to a protocol for modular design and construction of nucleic acid molecules.

An eighth aspect of the present invention relates to a method of adsorbing (chelating) a target molecule. This method involves providing a multivalent nucleic acid molecule of the present invention, wherein first and second nucleic acid elements bind the same target molecule at distinct sites. Upon contacting the target molecule with the nucleic acid molecule, the first and second nucleic acid elements bind the target molecule with sufficient affinity to adsorb (chelate) the target molecule.

The present invention utilizes a nucleic acid molecule to mimic the multi-functional aspects of proteins. Since the biological properties of a protein molecule depend on its physical interaction with other molecules, a basic, generic, and unambiguous description of a protein's function consists of a list of interactions or interacting partners with which it is involved, including interactions with other individuals of the same chemical identity. As shown in FIG. 1, these interactions would increase or decrease the stability, function, or both, of one, the other, or both interacting partners. More concrete, higher-order functions are emergent properties of these interactions. Global analyses of intracellular protein interaction revealed a highly heterogeneous “scale-free” topology, in which many proteins interacting with very few others coexist with a few densely connected “hubs” (Jeong et al., “Lethality and Centrality in Protein Networks,” Nature 411:41-42 (2001), which is hereby incorporated by reference in its entirety). This architecture implies that many proteins interact with more than three partners. If every protein possesses only one interacting site, the result is a collection of pairs connected by dyadic interactions and some orphans that do not interact with others. If proteins can only possess up to two sites, the most complex interaction pattern would be a chain and not a true network. Therefore, for a non-protein chemical to imitate protein function, the mimic should be able to accommodate at least two, but preferably three or more interacting sites without difficulty, a feature of nucleic acid constructs that has not been described previously.

The specific binding sites on proteins are often present on discrete modular domains as a result of evolutionary duplication or shuffling of their coding DNA sequences. Modules confer versatility in molecular construction and assist folding of biomacromolecules. Some typical protein modules have a stable core of β-sheets from which loops of polypeptide chain protrude and form binding sites for other molecules (Baron et al., “Protein Modules,” Trends Biochem. Sci. 16:13-17 (1991), which is hereby incorporated by reference in its entirety). For example, antibody molecules are composed of a typical type of α-sheet-based module, the “immunoglobulin fold.” Other similar modules include complement control module, fibronectin type 1 and type 3 modules, growth factor module, and kringle module. The present invention also seeks to mimic this kind of convenient and versatile modular framework for the presentation of various binding sites through changes to the protruding loops. However, it is impossible to re-create this kind of arrangement in a different chemical form (e.g. nucleotide polymer rather than amino acid polymer) by simple “blueprint copying.” To emulate this useful feature and achieve functional nucleic acid aptamer structures, the design principle was extracted and adapted to accommodate the nature of nucleic acids.

### BRIEF DESCRIPTION OF THE DRAWINGS

FIG. 1 is a schematic representation of protein function through protein-protein interactions. This figure illustrates the functional consequences of interactions and their modulation by different means.

FIG. 2 is an overview of the modular design and construction of the present invention. Two types of modules, structural or functional, and the protocol to connect them are depicted. The process of assembling modules according to the protocol is also illustrated. The circles in the modules signify the active sites of the functional elements. The dotted lines signify the fusion of complementary strands (indicated by arrows) in the protocol. In the assembled constructs, the circles signify the active sites, and thick black lines signify structural elements, which include inter alia three-way junctions, stems, and U-turns.

FIGS. 3A-C illustrate the process of constructing a functional protein mimic through the protocol of stem fusion. Referring to FIG. 3A, the predicted secondary structure of two full-length RNA aptamer clones are given. The structures were generated by the computer program Mfold. RA1-HSF (SEQ ID NO: 15) is an RNA aptamer against Drosophila heat shock factor generated in the inventors' laboratory; S1 (SEQ ID NO: 14) is a published RNA aptamer against streptavidin (Srisawat et al., “Streptavidin Aptamers: Affinity Tags for the Study of RNAs and Ribonucleoproteins,” RNA 7:632-641 (2001), which is hereby incorporated by reference in its entirety). It has been determined that in both cases the portion enclosed in the box with sequence represented in capital letters is sufficient to retain full aptamer activity. FIG. 3B shows a construct (SEQ ID NO: 17) designed by fusing together through stems one reduced RA1-HSF with two reduced S1's. This construct is analogous to an antibody that can be used in immuno assays, as depicted on the right side (SA stands for streptavidin). The secondary structure depicted here has been checked by Mfold (Zuker et al., “On Finding All Suboptimal Foldings of an RNA Molecule,” Science 244:48-52 (1989), which is hereby incorporated by reference in its entirety) to ensure the correct folded form of each aptamer components in this composite structure. FIG. 3C shows two antibody-like DNA constructs (SEQ ID NOS: 18, 19). The DNA version of streptavidin aptamer is taken from Bittker et al., “Nucleic Acid Evolution and Minimization by Nonhomologous Random Recombination,” Nat. Biotechnol. 20:1024-1029 (2002), which is hereby incorporated by reference in its entirety. The DNA three way junction is taken from van Buuren et al., “Solution Structure of a DNA Three-way Junction Containing Two Unpaired Thymidine Bases. Identification of Sequence Features that Decide Conformer Selection,” J. Mol. Biol. 304:371-383 (2000), which is hereby incorporated by reference in its entirety. The HSE is described in Xiao et al., “Cooperative Binding of Drosophila Heat Shock Factor to Arrays of a Conserved 5 bp Unit,” Cell 64:585-593 (1991), which is hereby incorporated by reference in its entirety. The Taq DNA polymerase aptamer is taken from Dang et al., “DNA Inhibitors of Taq DNA Polymerase Facilitate Detection of Low Copy Number Targets by PCR,” J. Mol. Biol. 264:268-278 (1996), which is hereby incorporated by reference in its entirety.

FIGS. 4A-B give examples of RNA three-way junctions that can play structural roles in a construct to organize functional modules. FIG. 4A shows a 2-D representation of the three-way junction that incorporates information, in particular strand and base pair orientations (SEQ ID NO: 20), from both predicted secondary structure and 3-D crystal structure of H. marismortui 5S rRNA (Ban et al., “The Complete Atomic Structure of the Large Ribosomal Subunit at 2.4 A Resolution,” Science 289:905-920 (2000), which is hereby incorporated by reference in its entirety). “F” designates the end of the strand in front of the plane; “B” designates the end of the strand behind the plane. The part represented is enclosed in the secondary structure and demarcated by thick bars in the 3-D structure. Both structures were taken from Ban et al., “The Complete Atomic Structure of the Large Ribosomal Subunit at 2.4 A Resolution,” Science 289:905-920 (2000), which is hereby incorporated by reference in its entirety. FIG. 4B gives two other three-way junctions, System D (SEQ ID NO: 21) and System F (SEQ ID NO: 22), described in Diamond et al., “Thermodynamics of Three-Way Multibranch Loops in RNA,” Biochemistry 40:6971-6981 (2001), which is hereby incorporated by reference in its entirety. They were shown to be thermodynamically stable and analogous to the structure in FIG. 4A. They differ only by one nucleotide as indicated by an arrow for System D.

FIGS. 5A-C illustrate predicted secondary structures of aptabodies for B52 detection. These aptabodies have a “di-dimer” configuration based on two three-way junctions fused together by a linker region. Each building block is indicated by a box. FIG. 5A gives the structure of aptabody B-S (SEQ ID NO: 23), which induces the proximity between B52 and streptavidin. The inset shows Mfold output for this sequence. FIGS. 5B and 5C give the structures of Aptabodies B-R (SEQ ID NO: 24) and T-S (SEQ ID NO: 25), respectively. They are built with the same building blocks as those of B-S, plus a pair of interacting RNA modules (Duconge et al., “In Vitro Selection Identifies Key Determinants for Loop-Loop Interactions: RNA Aptamers Selective for the TAR RNA Element of HIV-1,” RNA 5:1605-1614 (1999), which is hereby incorporated by reference in its entirety). While B-R is a specific aptabody against B52, T-S is a generic secondary aptabody for the detection of B-R-like primary aptabodies.

FIGS. 6A-C illustrate the construction and testing of aptabodies, which demonstrates that the aptabodies mirror the function of antibodies and are useful in immuno assay formats. FIG. 6A is a schematic representation of template construction. Separate templates for homodimers are made first for the testing of single specificity. The gray bars with arrow indicate the T7 promoter sequence included in the 5′ side of the templates. To make the templates for di-dimers, the dimer templates were digested with BamHI and ligated. FIG. 6B presents images of electrophoretic mobility shift assays, showing the three interactions involved in the aptabodies, each tested with a high (H) and a low (L) concentration. For the B52 aptamer BBS, the activity of the dimer construct was compared with that of the full-length aptamer clone #8. For the streptavidin aptamer S1, a reduced version, S1 con, was also tested. For the RNA-RNA interaction between TAR and its aptamer R-06, reciprocal binding assays with radiolable on either partner are shown. FIG. 6C shows the performance of aptabodies in two immuno-assays where antigens were presented on solid phases. Western blot analyses using both aptabodies and similar amount of monoclonal antibodies are shown in the left panel. The middle panel is a dot blot analysis measuring the sensitivity of aptabodies. The right panel shows immunofluorescence on Drosophila polytene chromosome stained with aptabody B-S and Texas-red conjugated streptavidin. Heat shock loci are indicated in the lower image. A regular immunofluorescence result can be found in FIG. 13B of U.S. Pat. No. 6,458,559 to Shi et al., which is hereby incorporated by reference in its entirety.

FIGS. 7A-C illustrate the design of dendritic scaffolds with four receptacles. FIG. 7A shows two alternative ways of fusing two three-way junctions based on their secondary structures (SEQ ID NOS: 26, 27). FIG. 7B illustrates how to control the orientation of non-stacking stems based on the information incorporated into the 2-D representation of three-way junctions illustrated in FIG. 4A. In particular, two dendritic scaffolds—I and II—each with four receptacle stems, are depicted, and the method of adding another three-way junction in either of two orientations to these scaffolds is illustrated. The arrows indicate the 5′→3′ direction of the strand, and numbers indicate base-pairs. FIG. 7C gives the sequence of the two scaffolds, I and II, depicted in FIG. 7B. N can be any nucleic acid. The four receptacles are designated 1 through 4.

FIGS. 8A-E depict ancillary functional modules that can be grafted to receptacles 1, 3, and 4 in combination to facilitate the in vivo delivery of an aptamer presented through receptacle 2. FIG. 8A illustrates the addition of a hammerhead ribozyme (SEQ ID NO: 36) to receptacle 1. A six-base pair insertion is included for polymerization of the coding units in a synthetic gene. In the depiction of linear sequence arrangement of both DNA polymerizing units and RNA cleavage products, parts of the ribozyme are indicated by boxes. The inset showing sequence and stacking relationships of the ribozyme from sTRSV are taken from Khvorova et al., “Sequence Elements Outside the Hammerhead Ribozyme Catalytic Core Enable Intracellular Activity,” Nature Structural Biology 10:708-712 (2003), which is hereby incorporated by reference in its entirety. FIG. 8B illustrates a method of making a homodimer: RNA-RNA proximity via tetraloop and its receptor (SEQ ID NO: 37) engrafted to receptacle 3. The sequences and computer models are taken from Jaeger et al., “TectoRNA: Modular Assembly Units for the Construction of RNA Nano-Objects,” Nucleic Acids Res. 29:455-463 (2001), which is hereby incorporated by reference in its entirety. A method of control dimer configuration through phasing of the RNA-RNA interaction is also depicted. FIG. 8C illustrates a method of making a heterodimer: RNA-RNA proximity via TAR Mal (SEQ ID NO: 38) and its aptamer R-06₂₄A54G (SEQ ID NO: 39) grafted respectively to receptacle 3 of different molecules. FIG. 8D illustrates a method to export the RNA transcript cleaved by the built-in ribozymes. The constitutive transport element (CTE) of Mason-Pfizer monkey virus (SEQ ID NO: 40) (Ernst et al., “Secondary Structure and Mutational Analysis of the Mason-Pfizer Monkey Virus RNA Constitutive Transport Element,” RNA 3:210-222 (1997), which is hereby incorporated by reference in its entirety), which binds to the protein TAP (NXF1), can be engrafted to receptacle 4. FIG. 8E illustrates a method to recruit the target of the aptamer to a particular promoter. An RNA ligand (SEQ ID NO: 41) that binds to MS2 coat protein can be grafted to receptacle 4 to induce the proximity between the aptamer target and MS2 coat protein. In a cell that expresses an MS2 coat protein-DNA binding domain fusion protein, the target of the aptamer will be recruited to the promoter recognized by the DNA binding domain of the fusion protein. The native version of MS2 coat protein ligand is depicted on the left. A U-to-C change is adopted in the construct according to the result of an in vitro selection experiment (Schneider et al., “Selection of High Affinity RNA Ligands to the Bacteriophage R17 Coat Protein,” J. Mol. Biol. 228:862-869 (1992), which is hereby incorporated by reference in its entirety).

FIGS. 9A-F illustrate the process of designing a specific construct using the building blocks depicted in FIGS. 7 and 8. The general layout of the “three-hybrid” system and the predicted secondary structure of the existing “RNA hybrid” are depicted in FIGS. 9A and 9B, respectively, both taken from Cassiday et al., “Yeast Genetic Selections to Optimize RNA Decoys for Transcription Factor NF-Kappa B,” Proc. Natl. Acad. Sci. USA 100:3930-3935 (2003), which is hereby incorporated by reference in its entirety), with minor modification. FIG. 9C depicts a construct design (SEQ ID NO: 42) using modules described herein to substitute the RNA hybrid and introduce additional features. All structural and functional elements are indicated by boxes and other annotations. An HSF aptamer and a NF-kappaB aptamer (Cassiday et al., “In Vivo Recognition of an RNA Aptamer by Its Transcription Factor Target,” Biochemistry 40:2433-2438 (2001), which is hereby incorporated by reference in its entirety) are arbitrarily placed at receptacle 2. FIG. 9D shows Mfold outputs for different constructs. In the upper left panel (SEQ ID NO: 42), receptacle 2 is filled by 20 undefined nucleotides. In the lower left panel (SEQ ID NO: 44), the undefined nucleotides are displaced by a NF-kappaB aptamer, αP50. In the upper right panel (SEQ ID NO: 54), receptacle 2 is filled by a tetraloop. In the lower right panel (SEQ ID NO: 43), the HSF aptamer, RA1-HSF, is inserted between the tetraloop and receptacle 2. FIGS. 9E-F illustrate two more variants of the construct shown in FIG. 9C. These contain aptamers to a general transcription factor, the TATA-binding protein (“TBP”), and therefore act as in situ functional probes for this factor. FIG. 9E (left panel) (SEQ ID NO: 45) is a predicted secondary structure of a construct containing two AptTBP-12 aptamers (formerly named #12 in Fan et al., “Probing TBP Interactions in Transcrition Initiation and Reinitiation with RNA Aptamers that Act in Distinct Modes,” PNAS 101(18):6934-6939 (2004), which is hereby incorporated by reference in its entirety) and one MS2 binding site. FIG. 9E (right panel) is a predicted secondary structure (SEQ ID NO: 52) of a precursor of the construct shown in FIG. 9E (left panel). This is an RNA transcript from a yeast RNA polymerase III promoter (RPR1) prior to cleavage by two cis-acting hammerhead ribozyme. FIG. 9F (left panel) (SEQ ID NO: 46) is a predicted secondary structure of a construct containing one AptTBP-101 aptamer (SEQ ID NO: 47) and two MS2 binding sites. FIG. 9F (right panel) is a predicted secondary structure (SEQ ID NO: 53) of a precursor of the construct shown in FIG. 9F (left panel). This is an RNA transcript from a yeast RNA polymerase III promoter (RPR1) prior to cleavage by two cis-acting hammer head ribozyme.

FIGS. 10A-B show enrichment of MGMs and RA1-HSF in two stages of in vitro evolution. The graphs in FIG. 10A show relative abundance of MGMs and RA1-HSF in different generations, detected by oligonucleotide probes in Southern dot-blot analyses of DNA pools. The MGM probe was described previously (Shi et al., “Evolutionary Dynamics and Population Control During In Vitro Selection and Amplification with Multiple Targets,” RNA 8(11):1461-70 (2002), which is hereby incorporated by reference in its entirety). FIG. 10B shows the affinity and specificity of RA1-HSF to Drosophila HSF. His-tagged HSF and two fusion constructs prepared from bacteria are used to measure the affinity in an electrophoretic mobility shift (“EMS”) assay. RNA transcribed from clone 14-8 is included as a control and showed no binding at the highest concentration of the three constructs used (H=His-HSF, G=GST-HSF, M=MBP-HSF).

FIGS. 11A-D illustrate the isolation of aptamers for discrete functional sites on the surface of TBP. FIG. 11A is a structural model representing the DNA.TBP.TFIIA.TFIIB quaternary complex, taken from Geiger et al., “Crystal Structure of the Yeast TFIIA/TBP/DNA Complex,” Science 272(5263):830-6 (1996), which is hereby incorporated by reference in its entirety. Human TFIIB was modeled with the crystal structure of the yeast tertiary complex. FIG. 11B is a predicted secondary structure of AptTBP-101, using mfold developed by Zuker (Zuker et al., “On Finding All Suboptimal Foldings of an RNA Molecule,” Science 244:48-52 (1989), which is hereby incorporated by reference in its entirety). Capital letters represent randomized region; lower cases signify the constant regions. The “true” aptamer moiety defined by mutagenesis is enclosed in the box. FIG. 11C show the results of an EMS assay with labeled RNA probes. AptTBP-12 and AptTBP-101 recognize the DNA site and the TFIIA site respectively. In FIG. 11D, inhibitory effects on RNA polymerase II dependent in vitro transcription by AptTBP-12 and AptTBP-101 is shown. The TBP concentration in the whole cell extract is around 20 nM. The concentration indicated is that of the aptamers.

FIG. 12 is a predicted secondary structure (SEQ ID NO: 48) of a chelating aptabody specific to the TATA-binding protein. This construct comprises one Streptavidin aptamer, S1, and two TBP aptamers binding to distinct sites on TBP, AptTBP-12, and AptTBP-101, as annotated.

FIG. 13 is a predicted secondary structure (SEQ ID NO: 49) of a supramolecular assembly-specific aptabody specific to the TATA DNA.TBP.TFIIB complex. This construct comprises one Streptavidin aptamer, S1, one TBP aptamer, AptTBP101, and one aptamer directed to TFIIB, AptB4, as annotated.

FIG. 14 depicts the secondary structure of the building-block aptamers in the form in which they were isolated from a combinatorial pool. The structures in the circles were confirmed by mutational studies to be the active aptamer moieties. Comparing these structures with corresponding parts annotated in FIGS. 12 and 13 demonstrates the successful preservation of these structures, and in turn functions thereof, in the new context of aptabodies.

### DETAILED DESCRIPTION OF THE INVENTION

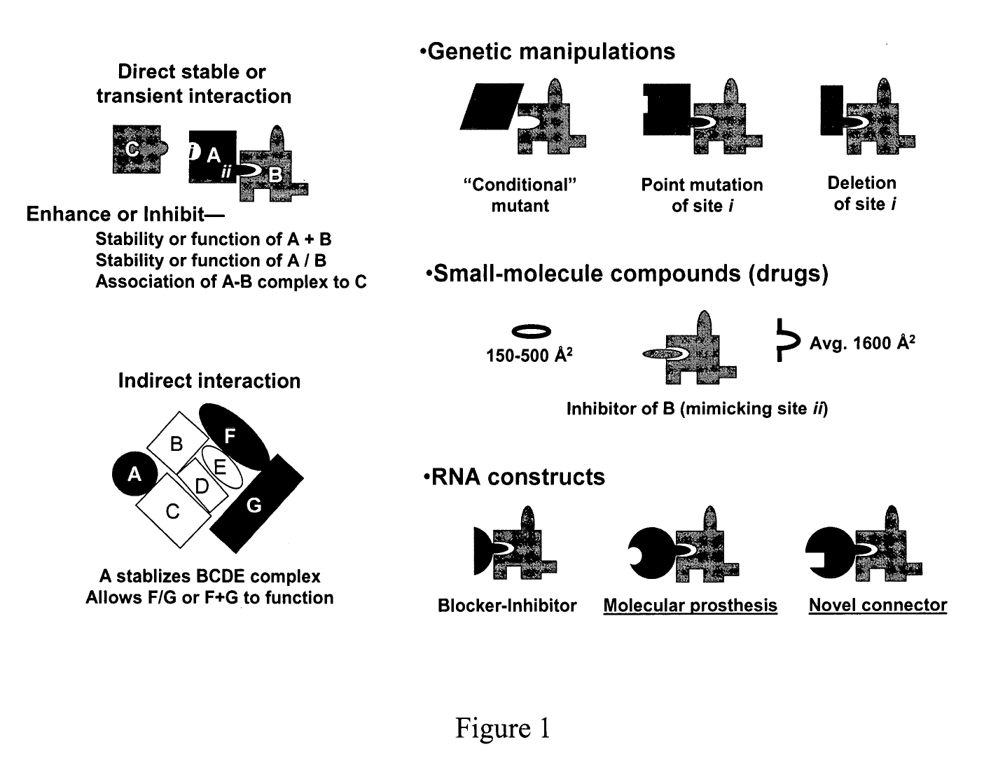

The present invention relates generally to the preparation and use of non-proteins, in particular nucleic acids, that imitate protein function. Proteins are able to play a predominant role in most biological processes largely because they can bear more than one (in many cases more than three) specific binding site for other molecules, including other proteins, which enables them to assemble into complexes or networks. For an experimentally tractable and objectively comparable definition of function for a particular protein, a “behavioral” approach has been taken, i.e., determining whether the non-protein is capable of imitating a given protein's behavior under conditions usually defined by the protein. As used herein, a non-protein can be considered a mimic of the protein if the non-protein is able to interact with at least two, but preferably all or substantially all partners of the protein with comparable affinity and specificity, and does not interact with any non-partner of the protein. Thus, a nucleic acid mimic of protein should have at least two, but preferably three or more, interacting sites in a single molecule that either coincide with the interacting pattern of a known protein (thus functioning as a prosthesis), or do not overlap with any known protein (“novel connector” in FIG. 1). In addition, a non-protein can be considered “protein-like” if the list of interactions of this non-protein includes at least two but preferably three or more partners, but the set of partners does not coincide with that of any known protein. “Protein-like” non-proteins should have a size similar to that of an average protein and should be able to function in biological environments. Its interacting partners should include at least one protein, so it can be incorporated into a protein network or assembly.

A nucleic acid molecule that can mimic protein function, or can otherwise be considered protein-like, includes both DNA and RNA, in both D and L enantiomeric forms, as well as derivatives thereof (including, but not limited to, 2′-fluoro-, 2′-amino, 2′O-methyl, 5′iodo-, and 5′-bromo-modified polynucleotides). Nucleic acids containing modified nucleotides (Kubik et al., “Isolation and Characterization of 2′fluoro-, 2′amino-, and 2′fluoro-/amino-modified RNA Ligands or Human IFN-gamma that Inhibit Receptor Binding,” J. Immunol. 159:259-267 (1997); Pagratis et al., “Potent 2′-amino, and 2′-fluoro-2′-deoxy-ribonucleotide RNA Inhibitors of Keratinocyte Growth Factor,” Nat. Biotechnol. 15:68-73 (1997), each of which is hereby incorporated by reference in its entirety) and the L-nucleic acids (sometimes termed Spiegelmers®) enantiomeric to natural D-nucleic acids (Klussmann et al., “Mirror-image RNA that Binds D-adenosine,” Nat. Biotechnol. 14:1112-1115 (1996); and Williams et al., “Bioactive and nuclease-resistant L-DNA Ligand of Vasopressin,” Proc. Natl. Acad. Sci. USA 94:11285-11290 (1997), each of which is hereby incorporated by reference in its entirety) are used to enhance biostability.

According to one embodiment, the nucleic acid molecule includes first and second nucleic acid elements that each bind a target molecule, and a three-way junction. The three-way junction is preferably formed of the same type of nucleic acid as the first and second nucleic acid elements, and operably links the first and second nucleic acid elements. In this embodiment, the nucleic acid molecule can interact with at least two target molecules, which can be the same or different.

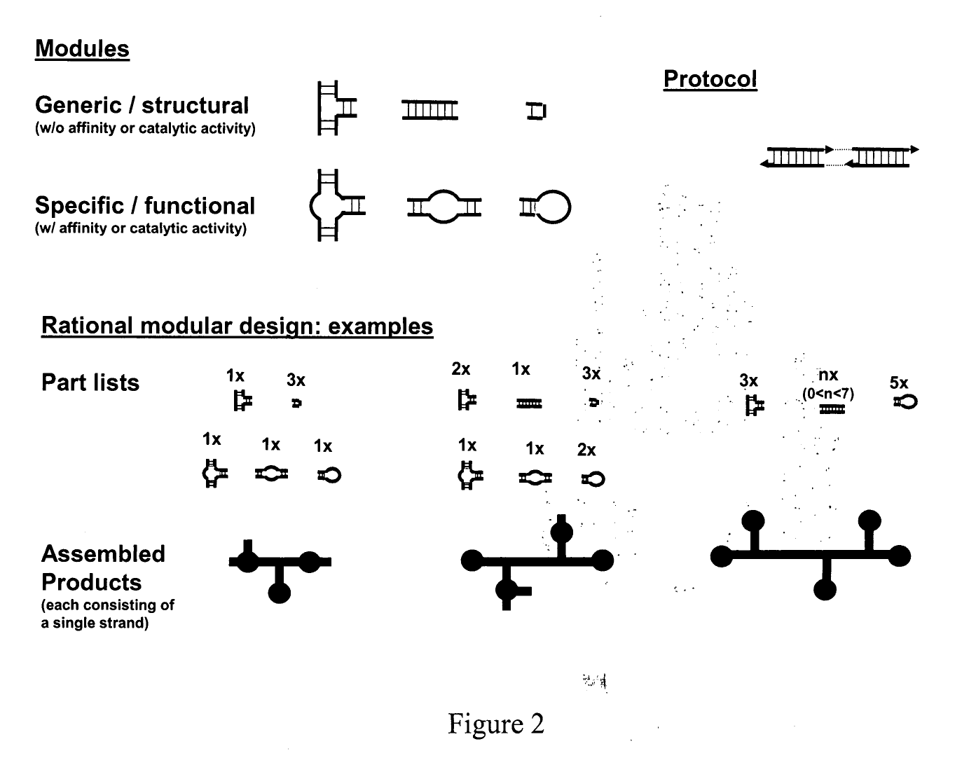

As shown in FIG. 2, the nucleic acid molecule can optionally include a third nucleic acid element operably connected to the three-way junction. The third nucleic acid element can also bind a target molecule. In this embodiment, the nucleic acid molecule can interact with three distinct target molecules, which can be the same or different.

Alternatively, the nucleic acid molecule can include a plurality of three-way junctions (n), wherein n is a positive integer that is greater than one, and a plurality of nucleic acid elements (≦n+2), each of which binds a target molecule. Each of the nucleic acid elements is operably linked to a three-way junction. Each of the three-way junctions is preferably of the same type of nucleic acid as the nucleic acid elements and is operably linked to another (i.e., at least one) of the plurality of three-way junctions.

When two or more three-way junctions are provided, the three-way junctions are connected to one another by a linker region that occupies a site on each three-way junction.

For example, as shown in one embodiment of FIG. 2, the nucleic acid molecule includes first, second, third, and fourth nucleic acid elements that each bind a target molecule. The first and second nucleic acid elements are operably linked by a first three-way junction, and the third and fourth nucleic acid elements are operably linked by a second three-way junction. The first and second three-way junctions are connected by a single linker region.

As yet another example, shown in another embodiment of FIG. 2, the nucleic acid molecule includes five nucleic acid elements that each bind a target molecule. The first and second nucleic acid elements, are operably linked by a first three-way junction; third and fourth nucleic acid elements are operably linked together by a second three-way junction; and a fifth nucleic acid element is operably linked to a third three-way junction located in between and operably coupled to the first and second three-way junctions via first and second linker regions, respectively.

Even greater numbers of nucleic acid elements can be present on nucleic acid molecules that contain larger numbers of three-way junctions coupled together via linker regions.

When employed, the linker region preferably contains as few nucleotides as possible. If formed of RNA, the linker region preferably contains not more than about 22 base pairs, i.e., achieves less than two helical turns. If formed of DNA, there is no structurally maximum length of the linker region, but preferably it is less than about 24 base pairs, more preferably less than about 12 base pairs. In principle, the linker region and other structural elements are preferably composed of the smallest number of bases that is sufficient to maintain correct folding pattern of the functional elements and confer desirable rigidity to the construct.

The three-way junctions used in preparing a nucleic acid molecule of the present invention are characterized structurally by the presence of three double-stranded nucleic acid chains that intersect, and three complementary base pairs (six nucleotides) that form the three-way junction. The three nucleic acid chains are preferably part of a single nucleic acid molecule containing stem and loop structures. Each stem preferably comprises two or more consecutive, canonical base-pairs between anti-parallel strands, and the junction region comprises either bases that do not participate in canonic pairing, or no bases at all. Suitable RNA three-way junctions include, without limitation, Loop A and accompanying 5S RNA (from H. marismortui 5S rRNA) (Ban et al., “The Complete Atomic Structure of the Large Ribosomal Subunit at 2.4 A Resolution,” Science 289:905:920 (2000), which is hereby incorporated by reference in its entirety), System D, or System F (Diamond et al., “Thermodynamics of Three-Way Multibranch Loops in RNA,” Biochemistry 40:6971-6981 (2000), which is hereby incorporated by reference in its entirety). Suitable DNA three-way junctions include, without limitation, TWJ1, J3CC, RPC2, and J3AA (van Buuren et al., “Solution Structure of a DNA Three-way Junction Containing Two Unpaired Thymidine Bases. Identification of Sequence Features that Decide Conformer Selection,” J. Mol. Biol. 304:371-383 (2000), which is hereby incorporated by reference in its entirety).

The nucleic acid elements can either have an affinity to and be able to bind a target molecule, have catalytic activity on a target molecule or the nucleic acid molecule itself, or have a role in accumulation, stability, oligomerization, or cellular localization of the nucleic acid molecule. The nucleic acid elements can be from about 10 nucleotides up to about 200 nucleotides in length, although larger or shorter nucleic acid elements can certainly be used. The nucleic acid elements are preferably between about 20 and about 120 nucleotides in length. In principle, the smallest functional form of the elements should be used.

One preferred type of nucleic acid element that has affinity to and can bind to a target molecule is an aptamer, such as an RNA aptamer or a DNA aptamer. Aptamers typically are generated and identified from a combinatorial library (typically in vitro) wherein a target molecule, generally although not exclusively a protein or nucleic acid, is used to select from a combinatorial pool of molecules, generally although not exclusively oligonucleotides, those that are capable of binding to the target molecule. The selected reagents can be identified as primary aptamers. The term “aptamer” includes not only the primary aptamer in its original form, but also secondary aptamers derived from (i.e., created by minimizing and/or modifying the structure of) the primary aptamer. Aptamers, therefore, behave as ligands, binding to their target molecule.

Identifying primary aptamers basically involves selecting aptamers that bind a target molecule with sufficiently high affinity (e.g., K_{d}=20-50 nM) and specificity from a pool of nucleic acids containing a random region of varying or predetermined length (Shi et al., “A Specific RNA Hairpin Loop Structure Binds the RNA Recognition Motifs of the Drosophila SR Protein B52,” Mol. Cell Biol. 17:1649-1657 (1997); Shi, “Perturbing Protein Function with RNA Aptamers,” Thesis, Cornell University, University Microfilms, Inc. (1997), which are hereby incorporated by reference in their entirety).

To identify primary aptamers of any particular target molecule, an established in vitro selection and amplification scheme, SELEX, can be used. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk and Gold, “Systematic Evolution of Ligands by Exponential Enrichment: RNA Ligands to Bacteriophage T4 DNA Polymerase,” Science 249:505-510 (1990), which are hereby incorporated by reference in their entirety.

Experimental data suggest that a pool of 1012 to 1015 different sequences seems to be comprehensive, i.e., contains nearly all chemically plausible RNA activities (Knight and Yarus, “Finding Specific RNA Motifs: Function in a Zeptomole World?” RNA 9:218-230 (2003), which is hereby incorporated by reference in its entirety). Commercial oligonucleotide synthesis generally yields more than 500 picomoles of the template at the 200 nmol synthesis scale. The synthetic oligodeoxynucleotide templates can be amplified by polymerase chain reaction (“PCR”) and then transcribed to generate the original RNA pool. Assuming that ten percent of the RNA molecules are free of chemical lesions that prevent second-strand synthesis and transcription, this pool would contain more than 3×10¹³ different sequences. In the inventors' laboratory, DNA and RNA pools containing up to 2.5×10¹⁶ different sequences have been generated and used in in vitro selection experiments. Because filter binding is applicable for most protein targets, it can be used as the partitioning device, although other suitable schemes can be used. To allow for exhaustive selection of all aptamers in a single pool, the iterative process described in U.S. patent application Ser. No. 10/173,368 to Shi et al. (which is hereby incorporated by reference in its entirety) can be used. The selected primary RNA aptamers can be cloned into any conventional subcloning vector and sequenced using any variation of the dideoxy method. Next, the secondary structure of each primary RNA aptamer can be predicted by computer programs such as MulFold (Jaeger et al., “Improved Predictions of Secondary Structures for RNA,” Proc. Natl. Acad. Sci. USA 86:7706-7710 (1989), and Zuker, “On Finding All Suboptimal Foldings of an RNA Molecule,” Science 244:48-52 (1989), which are hereby incorporated by reference in their entirety). Mutational studies can be conducted by preparing substitutions or deletions to map both binding sites on the RNA aptamer and its target molecule. See U.S. Pat. No. 6,458,559 to Shi et al., which is hereby incorporated by reference in its entirety.

Exemplary RNA aptamers are described in U.S. Pat. No. 5,270,163 to Gold et al., Ellington and Szostak, “In Vitro Selection of RNA Molecules that Bind Specific Ligands,” Nature 346:818-822 (1990), and Tuerk and Gold, “Systematic Evolution of Ligands by Exponential Enrichment: RNA Ligands to Bacteriophage T4 DNA Polymerase,” Science 249:505-510 (1990), which are hereby incorporated by reference in their entirety. Further exemplary RNA aptamers that bind to a Drosophila B52 splicing factor protein and to the Drosophila Heat Shock Factor (HSF) are described respectively in U.S. Pat. No. 6,458,559 to Shi et al., and in U.S. patent application Ser. No. 10/173,368 to Shi et al., each of which is hereby incorporated by reference in its entirety. Other known RNA aptamers include, without limitation, RNA ligands of T4 DNA polymerase, RNA ligands of HIV reverse transcriptase, RNA ligands of bacteriophage R17 coat protein, RNA ligands for nerve growth factor, RNA ligands of HSV-1 DNA polymerase, RNA ligands of Escherichia coli ribosomal protein S1, and RNA ligands of HIV-1 Rev protein (U.S. Pat. No. 5,270,163 to Gold et al., which is hereby incorporated by reference in its entirety); RNA ligands of Bacillus subtillus ribonuclease P (U.S. Pat. No. 5,792,613 to Schmidt et al., which is hereby incorporated by reference in its entirety); RNA ligands of ATP and RNA ligands of biotin (U.S. Pat. No. 5,688,670 to Szostak et al., which is hereby incorporated by reference in its entirety); RNA ligands of prion protein (Weiss et al., “RNA Aptamers Specifically Interact with the Prion Protein PrP,” J. Virol. 71(11):8790-8797 (1997), which is hereby incorporated by reference in its entirety); RNA ligands of hepatitis C virus protein NS3 (Kumar et al., “Isolation of RNA Aptamers Specific to the NS3 Protein of Hepatitis C Virus from a Pool of Completely Random RNA,” Virol 237(2):270-282 (1997); Urvil et al., “Selection of RNA Aptamers that Bind Specifically to the NS3 Protein of Hepatitis C Virus,” Eur. J. Biochem. 248(1):130-138 (1997); Fukuda et al., “Specific RNA Aptamers to NS3 Protease Domain of Hepatitis C Virus,” Nucleic Acids Symp. Ser. 37:237-238 (1997), which are hereby incorporated by reference in their entirety); RNA ligands of chloramphenicol (Burke et al., “RNA Aptamers to the Peptidyl Transferase Inhibitor Chloramphenicol,” Chem. Biol. 4(11):833-843 (1997), which is hereby incorporated by reference in its entirety); RNA ligands of the adenosine moiety of S-adenosyl methionine (Burke and Gold, “RNA Aptamers to the Adenosine Moiety of S-Adenosyl Methionine: Structural Inferences from Variations on a Theme and the Reproducibility of SELEX,” Nucleic Acids Res. 25(10):2020-2024 (1997), which is hereby incorporated by reference in its entirety); RNA ligands of protein kinase C (Conrad et al., “Isozyme-Specific Inhibition of Protein Kinase C by RNA Aptamers,” J. Biol. Chem. 269(51):32051-32054 (1994); Conrad and Ellington, “Detecting Immobilized Protein Kinase C Isozymes with RNA Aptamers,” Anal Biochem. 242(2):261-265 (1996), which are hereby incorporated by reference in their entirety); RNA ligands of subtilisin (Takeno et al., “RNA Aptamers of a Protease Subtilisin,” Nucleic Acids Symp. Ser. 37:249-250 (1997), which is hereby incorporated by reference in its entirety); RNA ligands of yeast RNA polymerase II (Thomas et al., “Selective Targeting and Inhibition of Yeast RNA Polymerase II by RNA Aptamers,” J. Biol. Chem. 272(44):27980-27986 (1997), which is hereby incorporated by reference in its entirety); RNA ligands of human activated protein C (Gal et al., “Selection of a RNA Aptamer that Binds to Human Activated Protein C and Inhibits its Protein Function,” Eur. J. Biochem. 252(3):553-562 (1998), which is hereby incorporated by reference in its entirety); and RNA ligands of cyanocobalamin (Lorsch and Szostak, “In vitro Selection of RNA Aptamers Specific for Cyanocobalamin,” Biochem. 33(4):973-982 (1994), which is hereby incorporated by reference in its entirety). Additional RNA aptamers are continually being identified and isolated by those of ordinary skill in the art.

The method of molecular design described above can also be applied to DNA without further modification. Single strand DNA molecules fold in a similar manner to RNA; double-stranded DNA usually assumes a B-form helix with 12 bp per turn. DNA three-way junctions have been studied, and rules of conformational selection in three-way junction in terms of stacking modes have been deduced with regards to base sequences close to the junction (van Buuren et al., “Solution Structure of a DNA Three-way Junction Containing Two Unpaired Thymidine Bases. Identification of Sequence Features that Decide Conformer Selection,” J. Mol. Biol. 304:371-383 (2000), which is hereby incorporated by reference in its entirety). DNA aptamers have been generated against a variety of targets including proteins (Gold et al., “Diversity of Oligonucleotide Functions,” Annu. Rev. Biochem. 64:763-797 (1995), which is hereby incorporated by reference in its entirety). A DNA aptamer of streptavidin is available and can be annexed to a double strand stem in the same manner described above (Bittker et al., “Nucleic Acid Evolution and Minimization by Nonhomologous Random Recombination,” Nat. Biotechnol. 20:1024-1029 (2002), which is hereby incorporated by reference in its entirety).

For DNA aptamers, a library of oligonucleotide sequences (sequence library) is synthesized comprising a randomized nucleotide region flanked by two defined PCR primer binding sites. The sequence library is amplified to yield double-stranded PCR products. To select for double-stranded DNA aptamers, the resultant population of double-stranded PCR products is then incubated (sans primer biotinylation and strand separation) with an identified target molecule. For preparation of single-stranded aptamers, the downstream PCR primer is biotinylated at the 5′ end and PCR products are applied to an avidin-agarose column. Single-stranded DNA oligonucleotides are recovered by elution with a weakly basic buffer. Resultant DNA strands are incubated with a selected target molecule either in solution or bound to a filter, chromatography matrix or other solid support. Nonbinding sequences are separated from binding sequences, e.g., by selective elution, filtration, electrophoresis, or alternative means of partitioning bound from free fractions. Typically, preselection and/or counterselection steps are included in the selection protocol to select against (i.e., remove or discard) nucleic acids that bind to nontarget substances (e.g., to a filter, gel, plastic surface, or other partitioning matrix) and/or irrelevant epitopes (e.g., the membrane portion of a membrane-associated receptor). Target-bound DNA sequences are then dissociated from the target and subjected to another round of PCR amplification, binding, and partitioning. After several rounds of enrichment and/or affinity maturation, the final amplification step may be performed with modified primers allowing subcloning into a plasmid restriction site and sequencing of target-binding positive clones.

Known DNA aptamers include, without limitation, those disclosed in Wang et al., “The Tertiary Structure of a DNA Aptamer which Binds to and Inhibits Thrombin Determines Activity,” Biochemistry 32:11285-11292 (1993); Huizenga et. al., “A DNA Aptamer that Binds Adenosine and ATP,” Biochemistry 34(2):656-665 (1995); Macaya et al., “Thrombin-Binding DNA Aptamer Forms a Unimolecular Quadruplex Structure in Solution,” Proc. Natl. Acad. Sci. USA 90:3745-3749 (1993); Mehedi et al., “Sialyllactose-Binding Modified DNA Aptamer Bearing Additional Functionality by SELEX,” Bioorg. Med. Chem. 12(5): 111-20 (2004); Saitoh et al., “Modified DNA Aptamers Against Sweet Agent Aspartame,” Nucleic Acids Res. Suppl. 2002(2):215-6 (2002); Moreno et al., “Selection of Aptamers Against KMP-11 Using Colloidal Gold During the SELEX Process,” Biochem. Biophys. Res. Commun. 308(2):214-8 (2003); Vianini et al., “In Vitro Selection of DNA Aptamers that Bind L-Tyrosinamide,” Bioorg. Med. Chem. 9(10):2543-8 (2001); Okazawa et al., “In Vitro Selection of Hematoporphyrin Binding DNA Aptamers,” Bioorg. Med. Chem. Lett. 10(23):2653-6 (2000); Wilson et al., “Isolation of a Fluorophore-Specific DNA Aptamer with Weak Redox Activity,” Chem. Biol. 5(11):609-17 (1998); which are hereby incorporated by reference in their entirety. Additional DNA aptamers are continually being identified and isolated by those of ordinary skill in the art.

Structurally, each aptamer sequence preferably has either a hairpin loop structure (i.e., with both a neck portion of various lengths that is characterized by a high degree of base-pairing and an apical loop portion that is characterized by non-paired bases of a target-binding sequence) or an internal loop structure (i.e., with a region characterized by non-paired bases positioned between two or more regions characterized by a high degree of pairing). Both the hairpin loop and internal loop structures are illustrated in FIG. 2 as functional modules.

Another preferred type of nucleic acid element is a catalytic element, such as a ribozyme, preferably a cis-acting ribozyme, as described infra.

Other preferred types of nucleic acid elements include various stabilization sequences, which can be incorporated into the nucleic acid molecule. A preferred stabilization sequence is an exonuclease-blocking sequence linked to an aptamer sequence. In particular, a stable tetra-loop near the 3′ end of the aptamer can be engineered. Because of its highly stacked and relatively inaccessible structure, the UUCG tetra-loop (Cheong et al., “Solution Structure of an Unusually Stable RNA Hairpin, 5′GGAC(UUCG)GUCC,” Nature 346:680-682 (1990), which is hereby incorporated by reference in its entirety) is used to stabilize nucleic acid molecules against degradation by 3′ exonucleases and to serve as a nucleation site for folding (Varani, “Exceptionally Stable Nucleic Acid Hairpins,” Annu. Rev. Biophys. Biomol. Struct. 24:379-404 (1995), which is hereby incorporated by reference in its entirety). Structurally, this type of loop is also used as a “U-turn” to close a stem region to make the strand continuous as a single molecular entity. Suitable U-turns for RNA include, without limitation, members of the UNCG and GNRA tetraloop families (Varani, “Exceptionally Stable Nucleic Acid Hairpins,” Annu. Rev. Biophys. Biomol. Struct. 24:379-404 (1995), which is hereby incorporated by reference in its entirety). Suitable U-turns for DNA include, without limitation, members of the GNRA tetraloop family (Varani, “Exceptionally Stable Nucleic Acid Hairpins,” Annu. Rev. Biophys. Biomol. Struct. 24:379-404 (1995), which is hereby incorporated by reference in its entirety).

In addition, the nucleic acid molecule can contain an “S35 motif” which yields a virtually closed structure resistant to nucleolytic degradation. The S35 motif, constructed by creating complementary 5′ and 3′ ends, has been shown to cause an over 100-fold increase in accumulation of a tRNA-ribozyme chimerical transcript in stably transduced cell lines (Thompson et al., “Improved Accumulation and Activity of Ribozymes Expressed from a tRNA-Based RNA Polymerase III Promoter,” Nucleic Acids Res. 23:2259-2268 (1995), which is hereby incorporated by reference in its entirety).

The nucleic acid molecule of the present invention can be directed to specific subcellular compartments to ensure that it will encounter the intended target and be concentrated in the organelle where the target resides. To this end, another preferred type of nucleic acid element is a location/localization element, which can be used to direct the nucleic acid molecule to specific subcellular locations. To export nucleic acid molecules from the nucleus, a specific nucleic acid sequence or structure, such as the Constitutive Transport Element of the type D retrovirus (Bray et al., “A Small Element from the Mason-Pfizer Monkey Virus Genome Makes Human Immunodeficiency Virus Type 1 Expression and Replication Rev-Independent,” Proc. Natl. Acad. Sci. USA 91:1256-1260 (1994); Ernst et al., “A Structured Retroviral RNA Element that Mediates Nucleocytoplasmic Export of Intron-Containing RNA,” Mol. Cell. Biol. 17:135-144(1997), which are hereby incorporated by reference in their entirety) can be appended to the nucleic acid molecule as a nucleic acid element. To direct nucleic acid molecules to other subcellular locations, specific proteins may be attached to the nucleic acid molecule to carry the nucleic acid molecule to its destiny. A suitable target ligand includes, without limitation, an MS2 coat protein ligand.

Target molecules capable of being bound by the nucleic acid elements may be natural or synthetic small molecules, macromolecules, supramolecular assemblies, or combinations thereof. Suitable target molecules include, without limitation, proteins, nucleic acids, liposaccharides, saccharides, lipoproteins, glycoproteins, and hydrocarbon polymers.

The nucleic acid elements on a single nucleic acid molecule can either (i) each bind distinct target molecules, (ii) each bind distinct regions of the same target molecule, or (iii) each bind the same target molecule. Thus, when the nucleic acid molecule contains a plurality of nucleic acid elements (≦n+2), each nucleic acid element may either bind a separate and distinct target molecule, or the same molecule as one or more of the other nucleic acid elements.

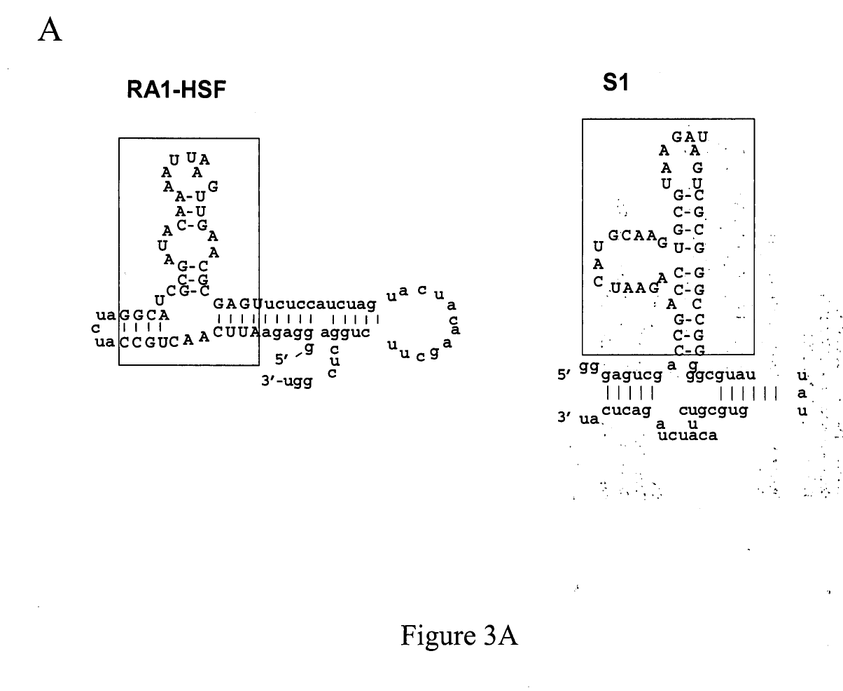

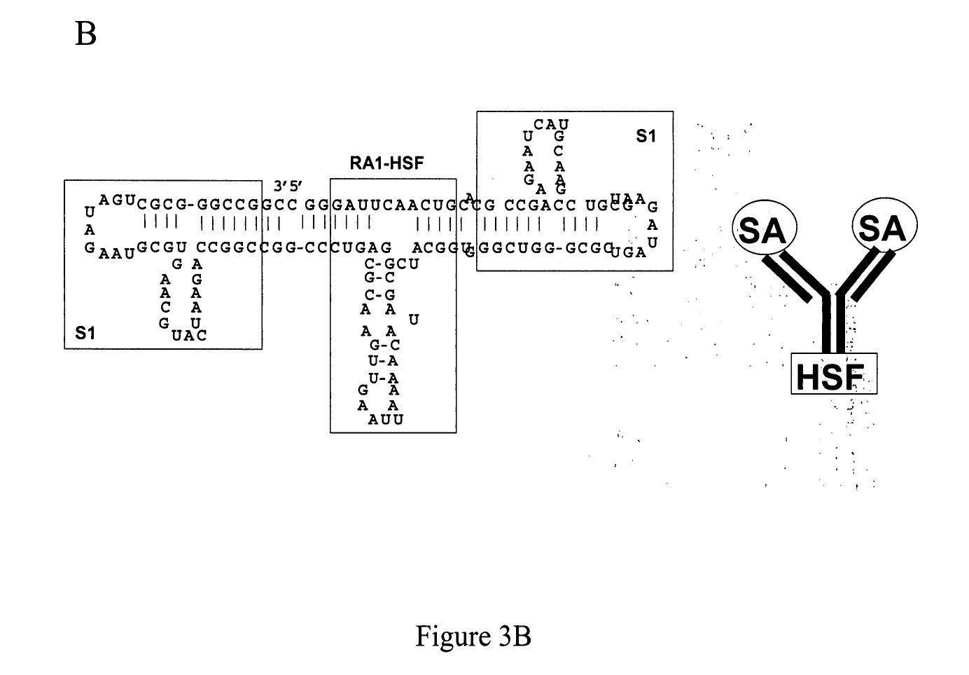

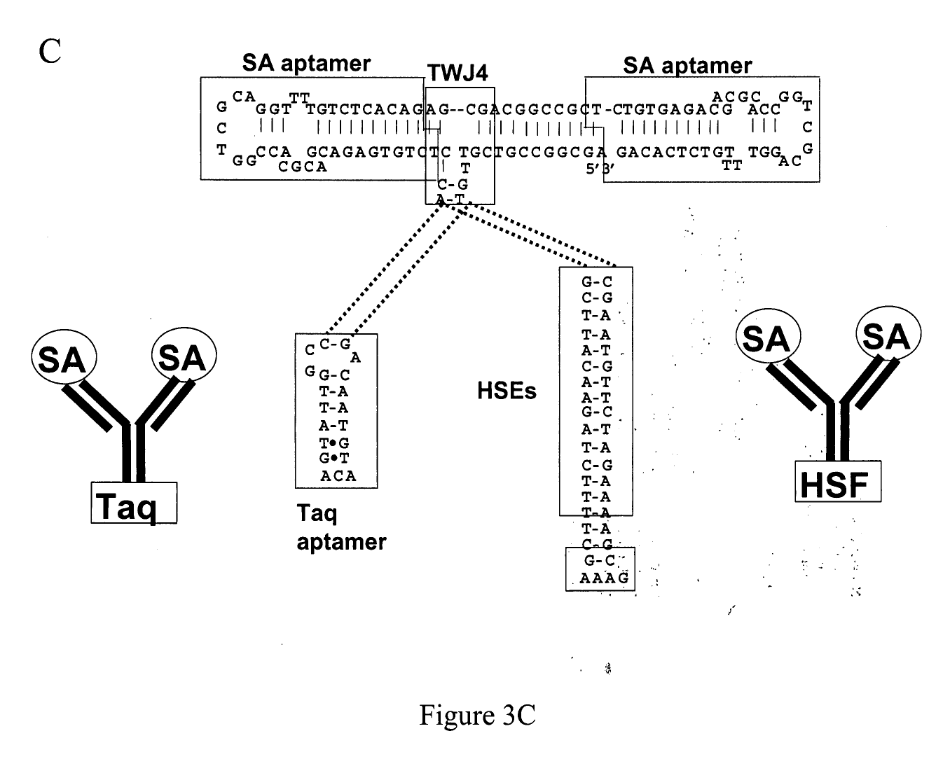

According to one embodiment, when the nucleic acid molecule contains first, second, and third nucleic acid elements operably linked by a single three-way junction, first and second nucleic acid elements can bind the same target molecule, and the third nucleic acid element can bind a different target molecule. One example of this embodiment is illustrated in FIG. 3B, which depicts an RNA molecule with three RNA aptamers, one of which binds Drosophila heat shock factor (RA1-HSF, FIG. 3A) (SEQ ID NO: 15) and two of which bind streptavidin (S1, FIG. 3A) (SEQ ID NO: 14). A second example of this embodiment is illustrated in FIG. 3C, which depicts two DNA molecules, each with three DNA aptamers. In one example, two streptavadin (“SA”) DNA aptamers are fused by a DNA three-way junction to a Taq DNA aptamer. In another example, two SA DNA aptamers are fused by a DNA three-way junction to an HSE DNA aptamer. The nucleic acid molecules depicted in FIGS. 3A-C are capable of inducing proximity between first and second target molecules.

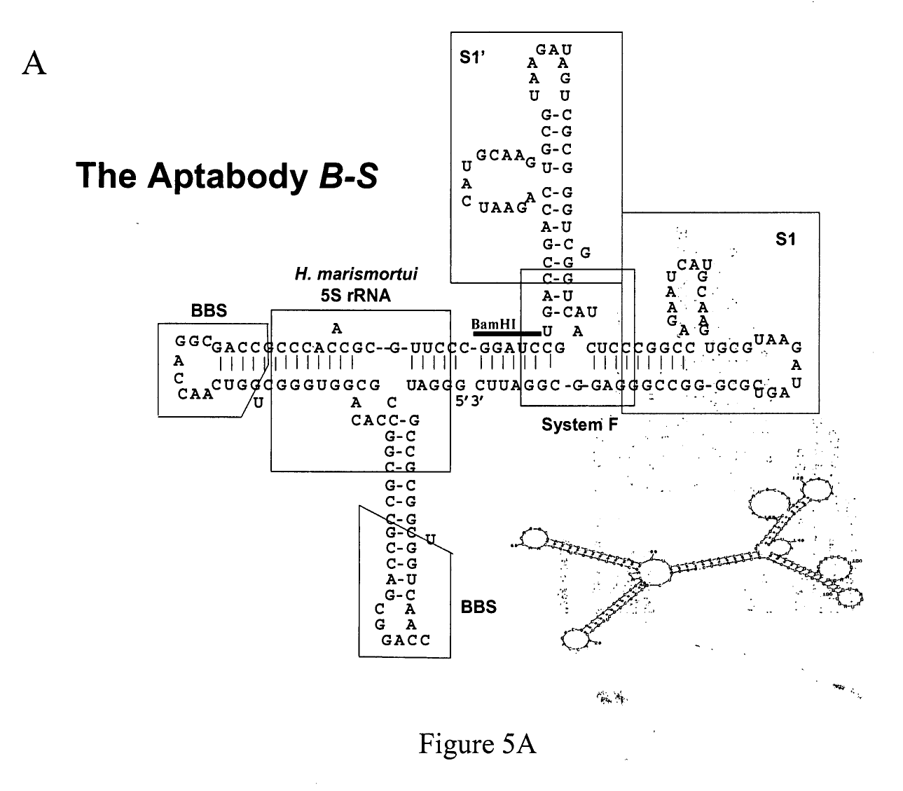

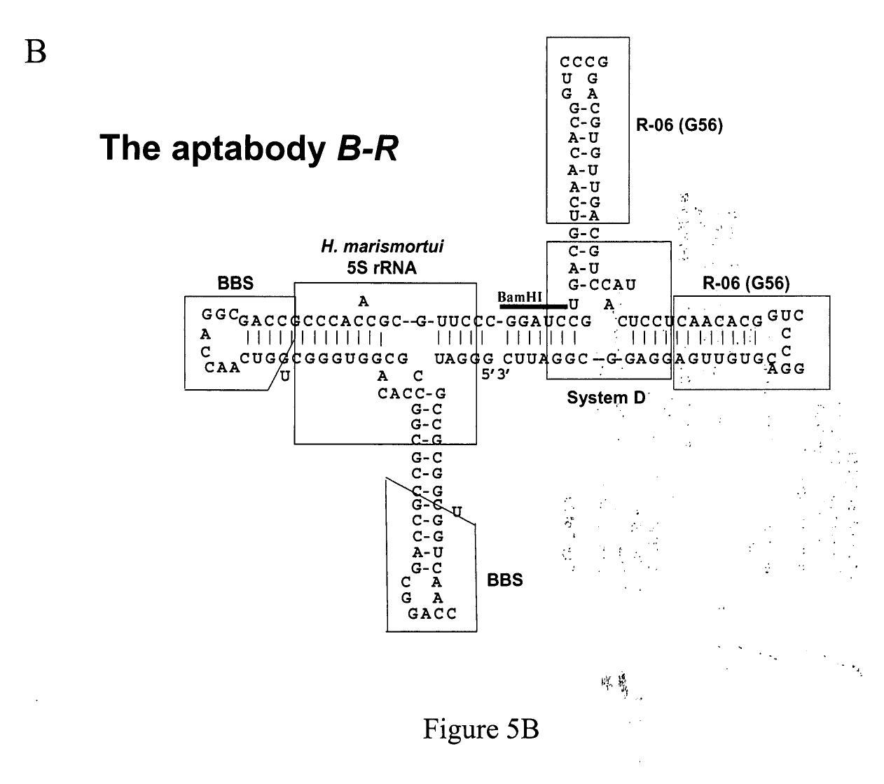

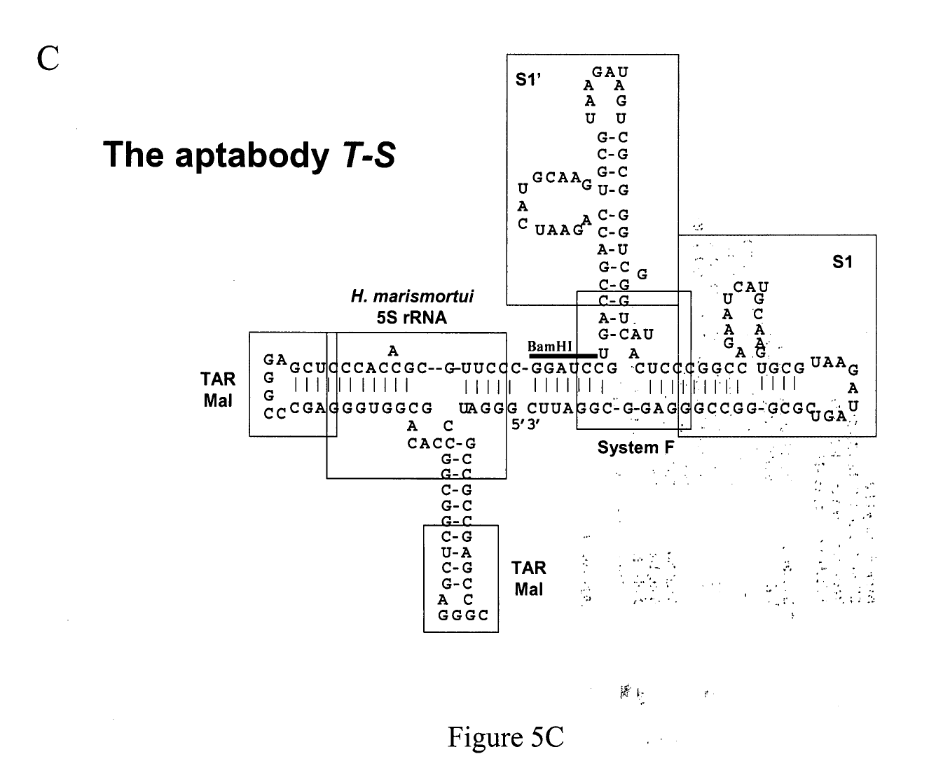

According to another embodiment, when the nucleic acid molecule contains a first, second, third, and fourth nucleic acid element, the first and second nucleic acid elements can each bind a first target molecule and the third and fourth nucleic acid elements can each bind a second target molecule that is different from the first target molecule. One example of this embodiment is illustrated in FIG. 5. The nucleic acid molecule has four aptamers, two of which bind B52 and are connected to a first three-way junction (the H. marismortui 5S RNA three-way junction), and the other two of which bind streptavidin and are connected to a second three-way junction (the System F three-way junction). This nucleic acid molecule, referred to as an “aptabody,” is capable of inducing proximity between the first and second target molecules.

When the nucleic acid molecule of the present invention is DNA, and the structure and sequence of the DNA molecule has been established, a constructed DNA molecule comprising the DNA sequence can be prepared. Preparation of the DNA molecule can be carried out by well-known methods of DNA ligation. DNA ligation utilizes DNA ligase enzymes to covalently link or ligate fragments of DNA together by catalyzing formation of a phosphodiester bond between the 5′ phosphate of one strand of DNA and the 3′ hydroxyl of another. Typically, ligation reactions require a strong reducing environment and ATP. The commonly used T4 DNA ligase is an exemplary DNA ligase in preparing the DNA molecule of the present invention. Once the DNA molecule of the present invention has been constructed, it can be incorporated into cells as described infra.

In contrast, when the nucleic acid molecule is RNA, and the structure and sequence of the RNA has been established, the RNA molecule can either be prepared synthetically or a DNA construct or an engineered gene capable of encoding such an RNA molecule can be prepared. Therefore, another aspect of the present invention relates to a DNA molecule and, more particularly, an engineered gene which encodes an RNA molecule of the present invention.

An engineered gene of the present invention includes a DNA sequence encoding an RNA molecule of the present invention, which DNA sequence is operably coupled to 5′ and/or 3′ regulatory regions as needed to ensure proper transcription of the RNA molecule in host systems.

Transcription of the DNA molecule of the present invention is dependent upon the presence of a promoter, which is a DNA sequence that directs the binding of RNA polymerase and thereby promotes RNA synthesis. The DNA sequences of eukaryotic promoters differ from those of prokaryotic promoters. Furthermore, eukaryotic promoters and accompanying genetic signals may not be recognized in or may not function in a prokaryotic system and, further, prokaryotic promoters are not recognized and do not function in eukaryotic cells.

Promoters vary in their “strength” (i.e., their ability to promote transcription). For the purposes of expressing the constructed DNA molecule or engineered gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

As described above, one type of regulatory sequence is a promoter located upstream or 5′ to the DNA sequence encoding the RNA molecule. Depending upon the desired activity, it is possible to select the promoter for not only in vitro production of the RNA molecule of the present invention, but also in vivo production in cultured cells or whole organisms, as described below. Because in vivo production can be regulated genetically, a preferable type of promoter is an inducible promoter which induces transcription of the DNA sequence in response to specific conditions, thereby enabling expression of the RNA molecule according to desired therapeutic needs (i.e., expression within specific tissues, or at specific temporal and/or developmental stages).

Preferred promoters for use in the engineered gene of the present invention include a T7 promoter, a SUP4 tRNA promoter, an RPR1 promoter, a GPD promoter, a GAL1 promoter, an hsp70 promoter, an Mtn promoter, a UAShs promoter, and functional fragments thereof. The T7 promoter is a well-defined, short DNA sequence that can be recognized and utilized by T7 RNA polymerase of the bactieriophage T7. The T7 RNA polymerase can be purified in large scale and is commercially available. The transcription reaction with T7 promoter can be conducted in vitro to produce a large amount of the RNA molecules of the present invention (Milligan et al., “Oligoribonucleotide Synthesis Using T7 RNA Polymerase and Synthetic DNA Templates,” Nucleic Acids Res. 15(21):8783-8798 (1987), which is hereby incorporated by reference in its entirety). The SUP4 tRNA promoter and RPR1 promoter are driven by RNA polymerase III of the yeast Saccharomyces cerevisiae, and suitable for high level expression of RNA less than 400 nucleotides in length (Kurjan et al., Mutation at the Yeast SUP4 tRNA^{tyr} Locus: DNA Sequence Changes in Mutants Lacking Supressor Activity,” Cell 20:701-709 (1980) and Lee et al., “Expression of RNase P RNA in Saccharomyces cerevisiae is Controlled by an Unusual RNA Polymerase III Promoter,” Proc. Natl. Acad. Sci. USA 88:6986-6990 (1991), each of which is hereby incorporated by reference in its entirety). The glyceraldehydes-3-phosphate dehydrogenase (GPD) promoter in yeast is a strong constitutive promoter driven by RNA polymerase II (Bitter et al., “Expression of Heterologous Genes in Saccharomyces cerevisiae from Vectors Utilizing the Glyceraldehyde-3-phosphate Dehydrogenase Gene Promoter,” Gene 32:263-274 (1984), which is hereby incorporated by reference in its entirety). The galactokinase (GAL1) promoter in yeast is a highly inducible promoter driven by RNA polymerase II (Johnston and Davis, “Sequences that Regulate the Divergent GAL1-GAL10 Promoter in Saccharomyces cerevisiae,” Mol. Cell. Biol. 4:1440-1448 (1984), which is hereby incorporated by reference in its entirety). The heat shock promoters are heat inducible promoters driven by the RNA polymerase II in eukaryotes. The frequency with which RNA polymerase II transcribes the major heat shock genes can be increased rapidly in minutes over 100-fold upon heat shock. The heat shock promoter used in the present invention can be a Drosophila hsp70 promoter, more preferably a portion of the Drosophila hsp70 promoter which is fully functional with regard to heat inducibility and designated heat inducible cassette, or Hic (Kraus et al., “Sex-Specific Control of Drosophila melanogaster Yolk Protein 1 Gene Expression is Limited to Transcription,” Mol. Cell. Biol. 8:4756-4764 (1988), which is hereby incorporated by reference in its entirety). Another inducible promoter driven by RNA polymerase II that can be used in the present invention is a metallothionine (Mtn) promoter, which is inducible to the similar degree as the heat shock promoter in a time course of hours (Stuart et al., “A 12-Base-Pair Motif that is Repeated Several Times in Metallothionine Gene Promoters Confers Metal Regulation to a Heterologous Gene,” Proc. Natl. Acad. Sci. USA 81:7318-7322 (1984), which is hereby incorporated by reference in its entirety). An additional promoter used in the present invention is a constructed hybrid promoter in which the yeast upstream activation sequence for the GAL1 genes was fused to the core Drosophila hsp70 promoter (Brand and Perrimon, “Targeted Gene Expression as a Means of Altering Cell Fates and Generating Dominant Phenotypes,” Development 118:401-415 (1993), which is hereby incorporated by reference in its entirety). This promoter is no longer activated by heat shock. Rather, it is activated by the yeast GAL4 protein, a transcription activator that is normally not present in Drosophila. The yeast GAL4 gene has been introduced into Drosophila, and is itself under a variety of transcriptional control in different fly lines.

Initiation of transcription in mammalian cells requires a suitable promoter, which may include, without limitation, β-globin, GAPDH, β-actin, actin, Cstf2t, SV40, MMTV, metallothionine-1, adenovirus E1a, CMV immediate early, immunoglobulin heavy chain promoter and enhancer, and RSV-LTR. Termination of transcription in eukaryotic genes involves cleavage at a specific site in the RNA which may precede termination of transcription. Also, eukaryotic termination varies depending on the RNA polymerase that transcribes the gene. However, selection of suitable 3′ transcription termination regions is well known in the art and can be performed with routine skill.

Spatial control of an RNA molecule can be achieved by tissue-specific promoters, which have to be driven by the RNA polymerase II. The many types of cells in animals and plants are created largely through mechanisms that cause different genes to be transcribed in different cells, and many specialized animal cells can maintain their unique character when grown in culture. The tissue-specific promoters involved in such special gene switching mechanisms, which are driven by RNA polymerase II, can be used to drive the transcription templates that code for the RNA molecules of the present invention, providing a means to restrict the expression of the RNA molecules in particular tissues.

For gene expression in plant cells, suitable promoters may include, without limitation, nos promoter, the small subunit ribulose bisphosphate carboxylase genes, the small subunit chlorophyll A/B binding polypeptide, the ³⁵S promoter of cauliflower mosaic virus, and promoters isolated from plant genes, including the Pto promoter itself. See C. E. Vallejos, et al., “Localization in the Tomato Genome of DNA Restriction Fragments Containing Sequences Homologous to the RRNA (45S), the major chlorophyll A/B Binding Polypeptide and the Ribulose Bisphosphate Carboxylase Genes,” Genetics 112: 93-105 (1986), which is hereby incorporated by reference in its entirety, and discloses the small subunit materials. The nos promoter and the 35S promoter of cauliflower mosaic virus are well known in the art.

In addition, the engineered gene may also include an operable 3′ regulatory region, selected from among those which are capable of providing correct transcription termination and polyadenylation of mRNA for expression in plant cells. A number of 3′ regulatory regions are known to be operable in plants. Exemplary 3′ regulatory regions include, without limitation, the nopaline synthase 3′ regulatory region (Fraley, et al., “Expression of Bacterial Genes in Plant Cells,” Proc. Nat'l. Acad. Sci. USA, 80:4803-4807 (1983), which is hereby incorporated by reference in its entirety) and the cauliflower mosaic virus 3′ regulatory region (Odell, et al., “Identification of DNA Sequences Required for Activity of the Cauliflower Mosaic Virus 35S Promoter,” Nature, 313(6005):810-812 (1985), which is hereby incorporated by reference in its entirety). Virtually any 3′ regulatory region known to be operable in plants would suffice for proper expression of the coding sequence of the engineered gene of the present invention.

To obtain high level expression of the RNA molecules of the present invention, the constructed DNA molecule or engineered gene can contain a plurality of monomeric DNA sequences ligated “head-to-tail,” each of which encodes an RNA molecule of the present invention. This is particularly useful for augmenting the number of RNA molecules produced during each transcriptional event. By plurality, it is intended that the number of monomeric DNA sequences be at least two, preferably at least four, more preferably at least eight, and most preferably at least twelve. Such tandemly arrayed sequences are known to be relatively stable in bacteria (Lindquist, “Varying Patterns of Protein Synthesis in Drosophila During Heat Shock: Implications for Regulation,” Dev. Biol. 77:463-479 (1980), which is hereby incorporated by reference in its entirety) and can persist for many generations in transgenic fly lines (Xiao and Lis, “A consensus Sequence Polymer Inhibits In Vivo Expression of Heat Shock Genes,” Mol Cell Biol 6:3200-3206 (1986); Shopland and Lis, “HSF Recruitment and Loss at Most Drosophila Heat Shock Loci is Coordinated and Depends on Proximal Promoter Sequences,” Chromosoma 105:158-171 (1996), which are hereby incorporated by reference in their entirety). This strategy should be applicable to other organisms. For example, long direct repeating sequences have been used in yeast (Robinett et al., “In Vivo Localization of DNA Sequences and Visualization of Largescale Chromatin Organization Using lac Operator/Repressor Recognition,” J. Cell Biol. 135:1685-700 (1996), which is hereby incorporated by reference in its entirety). It should be apparent to those of ordinary skill in the art, however, that the number of monomeric DNA sequences can vary for each application of the DNA molecule.

Depending upon the desired application and intended use for the DNA molecule, it is possible to produce homopolymers containing a plurality of substantially identical monomeric DNA sequences or copolymers containing a plurality of substantially different monomeric DNA sequences. It is also possible to produce copolymers, block polymers, or combinations thereof, that contain a plurality of substantially different monomeric DNA sequences. The RNA molecules produced from such a homopolymer are a single type. In contrast, the RNA molecules produced from such a copolymer, a block polymer, or a combination thereof, are different types. Thus, the plurality of monomeric DNA sequences can be substantially identical (i.e., producing substantially the same RNA molecule) or they can be substantially different (i.e., producing substantially different RNA molecules). When the plurality of monomeric DNA sequences are substantially different, the resulting RNA molecules can be directed to the same or to different target molecules.

When the DNA molecule encodes a plurality of monomeric DNA sequences, it is important that the resulting RNA transcript be cleaved into the individual mature RNA molecules of the present invention. To this end, it is particularly desirable for each of the plurality of monomeric DNA sequences to also encode a cis-acting ribozyme that can cleave the immature RNA transcript of the DNA molecule to yield multiple copies of the RNA molecule. Although any ribozyme sequence can be utilized, a hammerhead ribozyme sequence (Haseloff and Gerlach, “Simple RNA Enzymes with New and High Specific Endoribonucleases Activities,” Nature 334:585-591 (1988), which is hereby incorporated by reference in its entirety) is preferred because of its simplified and efficient structure. The sequence encoding the hammerhead ribozyme is incorporated into each of the plurality of monomeric DNA sequences, resulting in the hammerhead ribozyme being located at one end of each monomeric unit of the immature RNA transcript. The immature RNA transcript is self-cleaved by the cis-acting ribozyme to yield the mature RNA molecule. See U.S. Pat. No. 6,458,559 to Shi et al., which is hereby incorporated by reference in its entirety.

Once the DNA molecule or engineered gene of the present invention has been constructed, it can be incorporated into cells using conventional recombinant DNA technology. Generally, this involves inserting the DNA molecule or engineered gene into an expression system to which the DNA molecule or engineered gene is heterologous (i.e., not normally present). The heterologous DNA molecule or engineered gene is inserted into the expression system or vector in proper sense orientation. The vector contains the necessary elements for their persistent existence inside cells and for the transcription of the RNA molecule of the present invention.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and transfection, and replicated in cultures including prokaryotic organisms and eukaryotic cells grown in tissue culture.

Recombinant or engineered genes may also be introduced into viruses, such as vaccinia virus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference), pQE, pIH821, pGEX, pET series (see Studier et. al., “Use of T7 RNA Polymerase to Direct Expression of Cloned Genes,” Gene Expression Technology, vol. 185 (1990), which is hereby incorporated by reference in its entirety), pIIIEx426 RPR, pIIIEx426 tRNA (see Good and Engelke, “Yeast Expression Vectors Using RNA Polymerase III Promoters,” Gene 151:209-214 (1994), which is hereby incorporated by reference in its entirety), p426GPD (see Mumberg et al., “Yeast Vectors for the Controlled Expression of Heterologous Proteins in Different Genetic Background,” Gene 156:119-122 (1995), which is hereby incorporated by reference in its entirety), p426GAL1 (see Mumberg et al., “Regulatable Promoters of Saccharomyces cerevisiae: Comparison of Transcriptional Activity and Their Use for Heterologous Expression,” Nucleic Acids Research 22:5767-5768 (1994), which is hereby incorporated by reference in its entirety), pUAST (see Brand and Perrimon, “Targeted Gene Expression as a Means of Altering Cell Fates and Generating Dominant Phenotypes,” Development 118:401-415 (1993), which is hereby incorporated by reference in its entirety), and any derivatives thereof. Suitable vectors are continually being developed and identified.

A variety of host-vector systems may be utilized to express the RNA molecule-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, adeno-associated virus, retrovial vectors, etc.); insect cell systems infected with virus (e.g., baculovirus); and plant cells infected by bacteria or transformed via particle bombardment (i.e., biolistics). The expression elements of these vectors vary in their strength and specificities. Depending upon the host-vector system utilized, any one of a number of suitable transcription elements can be used.

Once the constructed DNA molecules or engineered genes encoding the RNA molecules, as described above, have been cloned into an expression system, they are ready to be incorporated into a host cell. Such incorporation can be carried out by the various forms of transformation, depending upon the vector/host cell system such as transformation, transduction, conjugation, mobilization, or electroporation. The DNA sequences are cloned into the vector using standard cloning procedures in the art, as described by Maniatis et al., Molecular Cloning: A Laboratory Manual, Cold Springs Laboratory, Cold Springs Harbor, N.Y. (1982), which is hereby incorporated by reference in its entirety. Suitable host cells include, but are not limited to, bacteria, yeast, mammalian cells, insect cells, plant cells, and the like. The host cell is preferably present either in a cell culture or in a whole living organism.

Plant tissues suitable for transformation include leaf tissue, root tissue, meristems, zygotic and somatic embryos, and anthers. It is particularly preferred to utilize embryos obtained from anther cultures.

The expression system of the present invention can be used to transform virtually any plant tissue under suitable conditions. Tissue cells transformed in accordance with the present invention can be grown in vitro in a suitable medium to control expression of a target molecule (e.g., a protein or nucleic acid) using an RNA molecule of the present invention. Transformed cells can be regenerated into whole plants such that the expressed RNA molecule regulates the function or activity of the target protein in the intact transgenic plants.

One approach to transforming plant cells and/or plant cell cultures, tissues, suspensions, etc. with a DNA molecule of the present invention is particle bombardment (also known as biolistic transformation) of the host cell. This technique is disclosed in U.S. Pat. Nos. 4,945,050, 5,036,006, and 5,100,792, all to Sanford, et al., which are hereby incorporated by reference in its entirety.

Another method of introducing the engineered gene of the present invention into a host cell is fusion of protoplasts with other entities, either minicells, cells, lysosomes, or other fusible lipid-surfaced bodies that contain the DNA molecule (Fraley, et al., “Expression of Bacterial Genes in Plant Cells,” Proc. Natl. Acad. Sci. USA, 80:4803-4807 (1983), which is hereby incorporated by reference in its entirety).

The DNA molecule of the present invention may also be introduced into the plant cells and/or plant cell cultures, tissues, suspensions, etc. by electroporation (Fromm, et al., “Expression of Genes Transferred into Monocot and Dicot Plant Cells by Electroporation,” Proc. Natl. Acad. Sci. USA, 82:5824 (1985), which is hereby incorporated by reference in its entirety).

In producing transgenic plants, the DNA construct in a vector described above can be microinjected directly into plant cells by use of micropipettes to transfer mechanically the recombinant DNA (Crossway, “Integration of Foreign DNA Following Microinjection of Tobacco Mesophyll Protoplasts,” Mol. Gen. Genetics, 202:179-85 (1985), which is hereby incorporated by reference in its entirety). The genetic material may also be transferred into the plant cell using polyethylene glycol (Krens, et al., “In Vitro Transformation of Plant Protoplasts with TI-Plasmid DNA,” Nature, 296:72-74 (1982), which is hereby incorporated by reference in its entirety).

One technique of transforming plants with the DNA molecules in accordance with the present invention is by contacting the tissue of such plants with an inoculum of a bacteria transformed with a vector comprising a DNA molecule or an engineered gene in accordance with the present invention. Generally, this procedure involves inoculating the plant tissue with a suspension of bacteria and incubating the tissue for 48 to 72 hours on regeneration medium without antibiotics at 25-28° C.

Bacteria from the genus Agrobacterium can be utilized to transform plant cells. Suitable species of such bacterium include Agrobacterium tumefaciens and Agrobacterium rhizogenes. Agrobacterium tumefaciens (e.g., strains C58, LBA4404, or EHA105) is particularly useful due to its well-known ability to transform plants.

Heterologous genetic sequences can be introduced into appropriate plant cells, by means of the Ti plasmid of A. tumefaciens or the R1 plasmid of A. rhizogenes. The Ti or R1 plasmid is transmitted to plant cells on infection by Agrobacterium and is stably integrated into the plant genome (Schell, “Transgenic Plants as Tools to Study the Molecular Organization of Plant Genes,” Science 237:1176-83 (1987), which is hereby incorporated by reference in its entirety).

After transformation, the transformed plant cells must be regenerated.

Plant regeneration from cultured protoplasts is described in Evans et al., Handbook of plant Cell Cultures, Vol. 1, MacMillan Publishing Co., New York (1983) and Vasil (ed.), Cell Culture and Somatic Cell Genetics of Plants, Acad. Press, Orlando, Vol. I (1984) and Vol. III (1986), which are hereby incorporated by reference.

It is known that practically all plants can be regenerated from cultured cells or tissues.

Mammalian cells suitable for carrying out the present invention include, without limitation, COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, NS-1 cells, and cells recovered directly from a mammalian organism.

In addition to in vitro transformation of mammalian cells, in vivo transformation can also be achieved. Thus, another aspect of the present invention relates to a transgenic non-human organism whose somatic and/or germ cell lines contain an engineered gene of the present invention (e.g., encoding an RNA molecule) which, upon expression thereof in the presence of a target molecule, modifies (e.g., inhibits) activity of the target molecule, wherein said modification (e.g., inhibition) is carried out in somatic and/or germ cells of the organism to rectify a condition associated with e.g., overexpression of the target molecule in somatic and/or germ cells of the organism. The target molecule can be any target used in the selection process, preferably a protein or nucleic acid. As described in U.S. Pat. No. 6,458,559 to Shi et al., which is hereby incorporated by reference in its entirety, RNA aptamer expression in a transgenic eukaryote can overcome a non-lethal phenotype associated with overexpression of a protein product.

The transgenic non-human organism is preferably a multicellular organism, such as a plant (as described supra), an animal, or an insect. The plant can be a monocot or a dicot. The animal can be a mammal, an amphibian, a fish, a reptile, or a bird. Preferred transgenic mammals of the present invention include sheep, goats, cows, dogs, cats, all non-human primates, such as monkeys and chimpanzees, and all rodents, such as rats and mice. Preferred insects include all species of Drosophila, particularly Drosophila melanogaster. It should be appreciated that the above-listed species or classes are only intended to be exemplary and, as such, are non-limiting.

J Procedures for making transgenic animals are well known. One means available for producing a transgenic animal (e.g., a mouse) is as follows: female mice are mated, and the resulting fertilized eggs are dissected out of their oviducts. The eggs are stored in an appropriate medium such as M2 medium (Hogan B. et al. Manipulating the Mouse Embryo, A Laboratory Manual, Cold Spring Harbor Laboratory (1986), which is hereby incorporated by reference in its entirety). A DNA or cDNA molecule is purified from a vector by methods well know in the art. As described above, inducible promoters may be fused with the coding region of the DNA to provide an experimental means to regulate expression of the transgene. Alternatively, or in addition, tissue specific regulatory elements may be fused with the coding region to permit tissue-specific expression of the transgene. The DNA, in an appropriately buffered solution, is put into a microinjection needle (which may be made from capillary tubing using a pipet puller), and the egg to be injected is put in a depression slide. The needle is inserted into the pronucleus of the egg, and the DNA solution is injected. The injected egg is then transferred into the oviduct of a pseudopregnant mouse (i.e., a mouse stimulated by the appropriate hormones to maintain pregnancy, but which is not actually pregnant), where it proceeds to the uterus, implants, and develops to term.

Alternatively, transgenic animals can be prepared by inserting a DNA molecule into a blastocyst of an embryo or into embryonic stem cells.

Transgenic organisms of the present invention are useful for modulating gene expression and protein activity in processes including, but not limited to, drug target validation, crop yield enhancement, fermentation, bioremediation, biodeterioration, and biotransformation.

Related aspects of the present invention involve methods of expressing an RNA molecule in a cell which include introducing either a DNA molecule of the present invention or an engineered gene of the present invention into a cell under conditions effective to express the RNA molecule. As described above, the conditions under which expression will occur are dependent upon the particular promoter or other regulatory sequences employed.

Another aspect of the present invention relates to a method of modifying the activity of a target molecule, either in vitro or in vivo (i.e., in a cell), which includes providing a nucleic acid molecule of the present invention in a cell, wherein the nucleic acid elements have affinity for one or more target molecules sufficient to modify activity of the one or more target molecule(s). This method can be carried out in vivo by directly introducing an RNA molecule or a DNA molecule into the cell, or by introducing into the cell (prior to the step of expressing) a DNA molecule (such as a DNA construct, engineered gene, or expression vector containing the same) encoding the RNA molecule. As described above, expression of the DNA molecule can be under the control of any one of a variety of regulatory sequences such as promoters, preferably inducible promoters. The cell can be in an in vitro environment, in an in vivo cell culture, or in vivo within an organism.

Modification of the activity of the target molecule(s) may include, without limitation, inhibiting the activity of the target molecule(s), promoting the activity of the target molecule(s), increasing the stability of the target molecule(s), and/or decreasing the stability of the target molecule(s).

To the extent that the activity of the target molecule (e.g., a protein) can be modified to achieve a therapeutic change in phenotype, the nucleic acid molecules can be used as therapeutic agents, alone or in combination with other therapeutic agents. For treatment of a patient, the nucleic acid molecules can be delivered directly as a part of a therapeutic composition. Alternatively, treatment of a patient may be carried out by delivering the nucleic acid molecules through methods of gene therapy. Nucleic acid molecules may be directly introduced into cells of tissues in vivo using delivery vehicles such as adenoviral vectors, retroviral vectors, DNA virus vectors, and colloidal dispersion systems. They may also be introduced into cells in vivo using physical techniques such as microinjection and electroporation or chemical methods such as coprecipitation and incorporation of nucleic acid into liposomes.

Adenovirus gene delivery vehicles can be readily prepared and utilized given the disclosure provided in Berkner, “Development of Adenovirus Vectors for the Expression of Heterologous Genes,” Biotechniques 6:616-627 (1988) and Rosenfeld et al., “Adenovirus-Epithelium In Vivo,” Science 252:431-434 (1991), WO 93/07283, WO 93/06223, and WO 93/07282, which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Adeno-associated viral gene delivery vehicles can be constructed and used to deliver into cells a nucleic acid molecule of the present invention. The use of adeno-associated viral gene delivery vehicles in vitro is described in Chatterjee et al., “Dual Target Inhibition of HIV-1 In Vitro by Means of an Adeno-Associated Virus Antisense Vector,” Science 258:1485-1488 (1992); Walsh et al., “Regulated High Level Expression of a Human Gamma-Globin Gene Introduced into Erythroid Cells by an Adeno-Associated Virus Vector,” Proc. Nat'l. Acad. Sci. USA 89:7257-7261 (1992); Walsh et al., “Phenotypic Correction of Fanconi Anemia in Human Hematopoietic Cells with a Recombinant Adeno-Associated Virus Vector,” J. Clin. Invest. 94:1440-1448 (1994); Flotte et al., “Expression of the Cystic Fibrosis Transmembrane Conductance Regulator from a Novel Adeno-Associated Virus Promoter,” J. Biol. Chem. 268:3781-3790 (1993); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); Miller et al., “Recombinant Adeno-associated Virus (rAAV)-Mediated Expression of a Human Gamma-Globin Gene in Human Progenitor-Derived Erythroid Cells,” Proc. Nat'l Acad. Sci. USA 91:10183-10187 (1994); Einerhand et al., “Regulated High-level Human Beta-Globin Gene Expression in Erythroid Cells Following Recombinant Adeno-Associated Virus-Mediated Gene Transfer,” Gene Ther. 2:336-343 (1995); Luo et al., “Adeno-Associated Virus 2-Mediated Gene Transfer and Functional Expression of the Human Granulocyte-Macrophage Colony-Stimulating Factor,” Exp. Hematol. 23:1261-1267 (1995); and Zhou et al., “Adeno-Associated Virus 2-Mediated Transduction and Erythroid Cell-Specific Expression of a Human β-Globin Gene,” Gene Ther. 3:223-229 (1996), which are hereby incorporated by reference in their entirety. In vivo use of these vehicles is described in Flotte et al., “Stable In Vivo Expression of the Cystic Fibrosis Transmembrane Conductance Regulator with an Adeno-Associated Virus Vector,” Proc. Nat'l Acad. Sci. USA 90:10613-10617 (1993); and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety.

Retroviral vectors which have been modified to form infective transformation systems can also be used to deliver into cells a nucleic acid molecule of the present invention. One such type of retroviral vector is disclosed in U.S. Pat. No. 5,849,586 to Kriegler et al., which is hereby incorporated by reference in its entirety.

Alternatively, a colloidal dispersion system can be used to deliver the nucleic acid molecule. Colloidal dispersion systems include macromolecule complexes, nanocapsules, microspheres, beads, and lipid-based systems including oil-in-water emulsions, micelles, mixed micelles, and liposomes. The preferred colloidal system of this invention is a lipid preparation including unilamaller and multilamellar liposomes.

Liposomes are artificial membrane vesicles that are useful as delivery vehicles in vitro and in vivo. It has been shown that large unilamellar vesicles (LUV), which range in size from about 0.2 to about 4.0 μm, can encapsulate a substantial percentage of an aqueous buffer containing nucleic acid molecules (Fraley et al., Trends Biochem. Sci. 6:77 (1981), which is hereby incorporated by reference in its entirety). In addition to mammalian cells, liposomes have been used for delivery of polynucleotides in yeast and bacterial cells. For a liposome to be an efficient transfer vehicle, the following characteristics should be present: (1) encapsulation of the nucleic acid molecules at high efficiency while not compromising their biological activity; (2) substantial binding to host organism cells; (3) delivery of the aqueous contents of the vesicle to the cell cytoplasm at high efficiency; and (4) accurate and effective expression of genetic information (Mannino et al., “Liposome Mediated Gene Transfer,” Biotechniques 6:682 (1988), which is hereby incorporated by reference in its entirety). In addition to such LUV structures, multilamellar and small unilamellar lipid preparations which incorporate various cationic lipid amphiphiles can also be mixed with anionic DNA molecules to form liposomes (Felgner et al., “Lipofection: A Highly Efficient, Lipid-Mediated DNA-Transfection Procedure,” Proc. Natl. Acad. Sci. USA 84(21): 7413 (1987), which is hereby incorporated by reference in its entirety).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and typically the presence of divalent cations. The appropriate composition and preparation of cationic lipid amphiphile/DNA formulations are known to those skilled in the art, and a number of references which provide this information are available (e.g., Bennett et al., “Considerations for the Design of Improved Cationic Amphiphile-based Transfection Reagents,” J. Liposome Research 6(3):545 (1996), which is hereby incorporated by reference in its entirety).

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. Examples of cationic amphiphilic lipids useful in formulation of nucleolipid particles for polynucleotide delivery include the monovalent lipids N-[1-(2,3-dioleoyloxy)propyl]-N,N,N,-trimethyl ammonium methyl-sulfate, N-[2,3-dioleoyloxy)propyl]-N,N,N-trimethyl ammonium chloride, and DC-cholesterol, the polyvalent lipids LipofectAMINE™, dioctadecylamidoglycyl spennine, Transfectam®, and other amphiphilic polyamines. These agents may be prepared with helper lipids such as dioleoyl phosphatidyl ethanolamine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization. The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Another aspect of the present invention relates to a method of using a multivalent nucleic acid aptamer to bring first and second target molecules into proximity of one another. This method involves providing a multivalent aptamer comprising first, second, and third aptamer sequences, wherein the first and second aptamer sequences are operably linked by a three-way junction, and the third aptamer sequence is operably linked to the first and second aptamer sequences, and wherein each of the first and second aptamer sequences is capable of binding the first target molecule, and the third aptamer sequence is capable of binding the second target molecule, which is different from the first target molecule. The method further involves exposing the multivalent nucleic acid aptamer to one or more samples that may contain the first and second target molecules. It is then determined whether the multivalent nucleic acid aptamer binds the first and second target molecules.

In one embodiment, the first target molecule is an antigen and the second target molecule is a labeled molecule, and the step of determining whether the nucleic acid molecule binds the first and second target molecules includes detecting the binding of the labeled molecule to the third aptamer sequence using an assay system.

Suitable assay systems for detecting the binding of the labeled molecule to the third aptamer sequence may include enzyme-linked immunoabsorbent assay, radioimmunoassay, gel diffusion precipitin reaction assay, immunodiffusion assay, agglutination assay, fluorescent immunoassay, protein A immunoassay, and immunoelectrophoresis assay.

In another embodiment, the first target molecule contains a DNA binding domain that targets the promoter region of a reporter gene, and the second target molecule contains a transcription activation domain, and the step of determining whether the multivalent nucleic acid aptamer binds the first and second target molecules includes detecting reporter gene expression. This type of assay for in vivo molecular proximity is referred to as “intracellular molecular-tethering assay.”

In yet another embodiment, the multivalent nucleic acid aptamer includes a fourth aptamer sequence operably linked by a second three-way junction to the third aptamer sequence, and a linker region that connects the first and second three-way junctions.

To the extent that nucleic acid molecules of the present invention can function as antibody-mimics, referred to as aptabodies, the nucleic acid molecules can be used to replace antibodies in any of the variety of immunological detection procedures. Although aptamers with single specificity have been used to fulfill molecular recognition needs in some assays and compared to antibodies in general (Jayasena et al., “Aptamers: An Emerging Class of Molecules that Rival Antibodies in Diagnostics,” Clin. Chem. 45:1628-1650 (1999), which is hereby incorporated by reference in its entirety), sensible comparison of performance can only be made between an aptamer and the Fab fragment of a monoclonal antibody to the same target or epitope. Indeed the specificity and affinity of aptamers are comparable to that of Fab fragments of antibodies to various antigens (Gold et al., “Diversity of Oligonucleotide Functions,” Annu. Rev. Biochem. 64:763-797 (1995), which is hereby incorporated by reference in its entirety). The utility of a real antibody in immunochemistry depends on at least two more features. First, it not only recognizes its antigen, but also interacts with labeled secondary reagents. Second, it has two Fab fragments that enhance its avidity to the antigen. Multivalency and multi-specificity together make antibodies efficient in bringing the signal or label to the vicinity of the antigen to signify the presence of the antigen (Harlow et al., Using Antibodies: A Laboratory Manual. Cold Springs Harbor, Cold Spring Harbor Laboratory Press (1999), which is hereby incorporated by reference in its entirety).

Herein it is demonstrated in vitro that two unrelated proteins from two different organisms can be bridged by a constructed RNA molecule derived from aptamers against these proteins. The same scheme can be used in vivo when similar RNA constructs are delivered via an engineered gene, as has been shown previously (U.S. Pat. No. 6,458,559 to Shi et al., and U.S. patent application Ser. No. 09/296,328 to Shi et al., each of which is hereby incorporated by reference in its entirety) and further by examples in this specification. Both multivalency and multi-specificity features of antibodies have been recapitulated in RNA molecules derived from aptamers. Their function has also been demonstrated in assay formats defined by antibodies.

Another aspect of the present invention relates to a method of adsorbing (chelating) a target molecule. This method involves providing a multivalent nucleic acid molecule of the present invention, wherein first and second nucleic acid elements (aptamers) bind the same target molecule at distinct sites. Upon contacting the target molecule with the nucleic acid molecule, the first and second nucleic acid elements bind the target molecule with sufficient affinity to adsorb (chelate) the target molecule. By way of example, in carrying out this method of the present invention, the multivalent nucleic acid molecule contains at least one three-way junction and two aptamers that bind a target molecule at different sites. Where multiple three-way junctions are used and four or more aptamers are used, each pair of aptamers can bind the same target molecule or different target molecules.

Thus, a further aspect of the present invention relates to a method of detecting the presence of a target molecule in a sample. This method involves exposing a nucleic acid molecule of the present invention to a sample under conditions effective to allow binding of a target molecule in the sample by the nucleic acid molecule and determining whether the nucleic acid molecule has bound to the target molecule. Determining whether the nucleic acid molecule has bound to the target molecule preferably involves detecting any reaction which indicates that the target molecule is present in the sample using an assay system. Suitable assay systems are described above.

Another aspect of the present invention relates to an RNA scaffold that can be assembled to have a predefined structure, which structure can be modified to include various functional modules. The RNA scaffold includes, at a minimum, first and second RNA receptor regions operably linked by a three-way junction. The first and second RNA receptor regions each comprise a stem defined by at least two sets of consecutive, canonic paired bases.

Alternatively, the RNA scaffold can include a plurality of three-way junctions (n), wherein n is a positive integer greater than 1, and a plurality of receptor regions (≦n+2), wherein each of the receptor regions is operably linked to a three-way junction. Each receptor region contains a structure as described above, and each three-way junction is operably linked to another (i.e., at least one) of the plurality of three-way junctions by a linker region.

The RNA scaffold preferably contains, in addition to the first and second receptor regions and first three-way junction, third and fourth RNA receptor regions operably linked by a second three-way junction, where the second three-way junction is joined to the first three-way junction by a linker region. Each of the first, second, third, and fourth RNA receptor regions contains a structure as described above.

The RNA scaffold can also include an RNA structure, formed by the termini thereof, that is resistant to exonuclease digestion.

Yet another aspect of the present invention relates to a method for modular design and construction of nucleic acid molecules. This method involves providing one or more structural nucleic acid modules and one or more functional nucleic acid modules. At least one of each of the structural and functional nucleic acid modules is combinatorially joined to form a single molecular entity according to a protocol for modular design and construction of nucleic acid molecules.

Modular design and construction of molecules constitutes the development of submolecular modules and protocols (FIG. 2). Modules are parts, components, or subsystems with identifiable interface to other modules. They maintain their identity when isolated or rearranged, and can be evolved somewhat independently. As illustrated by the examples herein, use of modules facilitates simplified, reduced, or abstracted modeling, and functional or variational description, in addition to procedural description. Protocols are rules or constraints on allowed interfaces and interconnections that facilitate modularity and simplify modeling, abstraction, and verification. They also facilitate independent evolvability of components and systems, and addition of new protocols. The particular configuration comprising the dendritic RNA scaffold and its accompanying receptor regions makes it possible to organize and present various functional modules with the help of structural modules.

Functional modules have known affinity to known targets or have known catalytic functions. The functional modules contain functional “loops” in association with different structural elements. Each “loop” is defined by a single function without regard to structure, which, in addition to possible canonical base pairings, usually have internal structure involving non-canonical base-base, backbone-backbone, and base-backbone interactions that are not predictable by common secondary structure prediction algorithms. In FIG. 2, only the simplest cases of such functional loops without specified internal structures are depicted, and include, without limitation, (1) functional apical loops and the associated stem “neck”; (2) functional internal loops embedded in a stem that may or may not be participating in the functioning; and (3) functional internal loops constituting the strand-exchange junction of three or more stems.

While the structural modules generally do not have the functions associated with the functional modules, for them, structural information beyond that obtainable from common secondary structure prediction algorithms is available. As illustrated in FIG. 2, three types of structural modules include, without limitation, (1) multi-branch junctions with known structures, in particular three-way junctions; (2) stems having complementary strands that form A-form double helix if the nucleic acid is RNA or B-form double helix if the nucleic acid is DNA; and (3) stable, small U-turns with known structure, such as hairpins with tetra-loop.

A single protocol of stem fusion can be applied one or more times in the process of design, allowing for the combinatorial joining of modules together in a single molecular entity.

RNA molecules fold in a process of hierarchical nature. Stable secondary structural elements fold on a fast, microsecond time scale, which determines formation of tertiary contacts (Brion et al., “Hierarchy and Dynamics of RNA Folding,” Annu. Rev. Biophys. Biomol. Struct. 26:113-137 (1997), which is hereby incorporated by reference in its entirety). Unlike proteins, RNA secondary structure formation, which is driven by stacking between contiguous base pairs, involves significantly larger amount of energy than that involved in tertiary interactions (Tinoco et al., “How RNA Folds,” J. Mol. Biol. 293:271-281 (1999), which is hereby incorporated by reference in its entirety). As a result, the basic properties of the conformational energy landscape of an RNA molecule can be understood at the level of secondary structures, and a secondary structure predicted by a free energy minimization algorithm can serve as a starting point for tertiary structural design.

In the present invention, a two-dimensional representation of three-way junctions that incorporate the information of both predicted secondary structure and real tertiary structure, based on which, a set of dendritic scaffolds can be constructed, is presented. Different functional RNA modules can be engrafted to the RNA scaffold stems by fusion of double-strand stems to those exiting from the multi-branch junctions, thus forming different constructs with their functional modules well-exposed to the solvent. Formally, the minimal case of a dendritic structure is a three-way junction. Multi-branch junctions of RNA or DNA with more than 3 stems converging to a single loop are usually less stable due to branch migration and other factors. Stable multi-branch junctions with more than 3 exiting stems can be generated by fusing multiple three-way junctions. The minimal case of this agglomeration is a 4-receptacle scaffold made by fusing two three-way junctions (via a linker region). Any RNA structural or functional module that has a helical stem, including aptamers and additional multi-branch junctions, can be annexed to the helices exiting from such a core. Thus “stem fusion” serves as a protocol that connects all modules, structural and functional. A typical RNA stem assumes a structure of A-form helix, and the optimal length for a regular A-form helical turn is 11+/−1 bp (Jaeger et al., “TectoRNA: Modular Assembly Units for the Construction of RNA Nano-Objects,” Nucleic Acids Res. 29:455-463 (2001), which is hereby incorporated by reference in its entirety). Therefore, the length of a stem can be used to roughly determine the direction of the non-stacking branches. As shown by the examples, the versatile connection between modules in this system generates reusable parts that are portable between species and systems.

The functional modules may serve a variety of functions, such as accumulation of the molecule, stability of the molecule, aptamer presentation, oligomerization, transportation of the molecule, or localization of the molecule. Exemplary types of functional modules include, without limitation, aptamers, catalytic elements, stabilization elements, location/localization elements, and target ligands.

In one embodiment, the scaffold has been functionalized to contain a cis-acting ribozyme coupled to a first receptor region, an RNA aptamer coupled to a second receptor region, a tetraloop and its receptor coupled to a third receptor region, and a constitutive transport element coupled to a fourth receptor region.

In another embodiment, the scaffold has been functionalized to contain a cis-acting ribozyme coupled to a first receptor region, an RNA aptamer coupled to a second receptor region, a tetraloop and its receptor coupled to a third receptor region, and an MS2 coat protein ligand coupled to a fourth receptor region.

In yet another embodiment, the scaffold has been functionalized to contain a cis-acting ribozyme coupled to a first receptor region, an RNA aptamer coupled to a second receptor region, a TAR element or an aptamer of TAR element coupled to a third receptor region, and a constitutive transport element coupled to a fourth receptor region.

In still another embodiment, the scaffold has been functionalized to contain a cis-acting ribozyme coupled to a first receptor region, an RNA aptamer coupled to a second receptor region, a tectoRNA module coupled to a third receptor region, and an MS2 coat protein ligand coupled to a fourth receptor region.

The scaffolds can be encoded by a DNA construct or engineered gene, as described above, and either expressed in an ex vivo host cell or in a transgenic non-human organism, also as described above.

These aspects of the present invention are further illustrated by the examples below.

### EXAMPLES

The following examples are provided to illustrate embodiments of the present invention, but they are by no means intended to limit its scope.

### Example 1

### Proteins and Antibodies

ImmunoPure® streptavidin, horseradish peroxidase conjugated streptavidin, and Texas Red® conjugated streptavidin were purchased from Pierce Biotechnology (Rockford, Ill.). Full-length B52 protein was prepared from a baculovirus expression system as described previously (Shi et al., “A Specific RNA Hairpin Loop Structure Binds the RNA Recognition Motifs of the Drosophila SR Protein B52,” Mol. Cell Biol. 17:1649-1657 (1997), which is hereby incorporated by reference in its entirety). His-tagged B52-RRMs and His-tagged dHSF were cloned in a pET vector (Novagen, Madison, Wis.), expressed in BL21 cells, and purified using Ni-NTA Superflow matrix (Qiagen, Valencia, Calif.). The monoclonal antibody By32 was described in Champlin et al., “Characterization of a Drosophila Protein Associated with Boundaries of Transcriptionally Active Chromatin,” Genes and Development 5:1611-1621 (1991), which is hereby incorporated by reference in its entirety. The anti-His antibody was purchased from Qiagen (Valencia, Calif.). The antimouse IgG antibody was purchased from Jackson ImmunoResearch Laboratories (West Grove, Pa.).

### Example 2

### Oligonucleotides

The aptabodies (i.e., aptamer-containing antibody mimics) were prepared by in vitro transcription (see infra) from templates made from the following oligonucleotides:

BBS-5′, which has a nucleotide sequence corresponding to SEQ ID NO: 1 as follows:

| | |
|---|---|
| GTAATACGAC TCACTATAGG GATCGCCGCG GCTGGTCAAC CAGGCGACCG CCGCGGCCAC | 60 |
| AGCGGTGGGC TGGTCA | 76 |

BBS-3′, which has a nucleotide sequence corresponding to SEQ ID NO: 2 as follows:

| | |
|---|---|
| GAATCCCGAA GGATCCGGGA ACGCTGGTGG GCGGTCGCCT GGTTGACCAG CCCACCGCTG | 60 |
| TGGCCGCGGC GGTCGCCT | 78 |

SAa-5′, which has a nucleotide sequence corresponding to SEQ ID NO: 3 as follows:

| | |
|---|---|
| GTAATACGAC TCACTATAGG ATCCGTGACC GACCAGAATC ATGCAAGTGC GTAAGATAGT | 60 |
| CGCGGGTCGG GTCATACTCC | 80 |

SAa-3′, which has a nucleotide sequence corresponding to SEQ ID NO: 4 as follows:

| | |
|---|---|
| GAATCCGCCT CCCGGCCCGC GACTATCTTA CGCACTTGCA TGATTCTGGC CGGGAGTATG | 60 |
| ACCCGACCCG CGACTATCTT | 80 |

TAR Mal-5′, which has a nucleotide sequence corresponding to SEQ ID NO: 5 as follows:

| | |
|---|---|
| GTAATACGAC TCACTATAGG GATCGCCGCC GAGCCCGGGA GCTCGGCGGC CACAGCGGTG | 60 |
| GGAGC | 65 |

TAR Mal-3′, which has a nucleotide sequence corresponding to SEQ ID NO: 6 as follows:

| | |
|---|---|
| GAATCCCGAA GGATCCGGGA ACGCTGGTGG GAGCTCCCGG GCTCCCACCG CTGTGGCCGC | 60 |

R-06-5′, which has a nucleotide sequence corresponding to SEQ ID NO: 7 as follows:

| | |
|---|---|
| GTAATACGAC TCACTATAGG ATCCGTGACG TCAACACGGT CCCGGACGTG TTGACGTCCA | 60 |
| TA | 62 |

R-06-3′, which has a nucleotide sequence corresponding to SEQ ID NO: 8 as follows:

| | |
|---|---|
| GAATCCGCCT CCTCAACACG TCCGGGACCG TGTTGAGGAG TATGGACGTC AACACGTCCG | 60 |

A reduced S1 construct is made by using the following two oligonucleotides:

5′S1cons, which has a nucleotide sequence corresponding to SEQ ID NO: 9 as follows:

| | |
|---|---|
| GTAATACGAC TCACTATAGG GACCGACCAG AATCATGCAA | 45 |
| GTGCG | |

3′S1cons, which has a nucleotide sequence corresponding to SEQ ID NO: 10 as follows:

| | |
|---|---|
| CCCGGCCCGC GACTATCTTA CGCACTTGCA TGATTCTGGT | 45 |
| CGGTC | |

### Example 3

### Aptabody Templates and RNAs

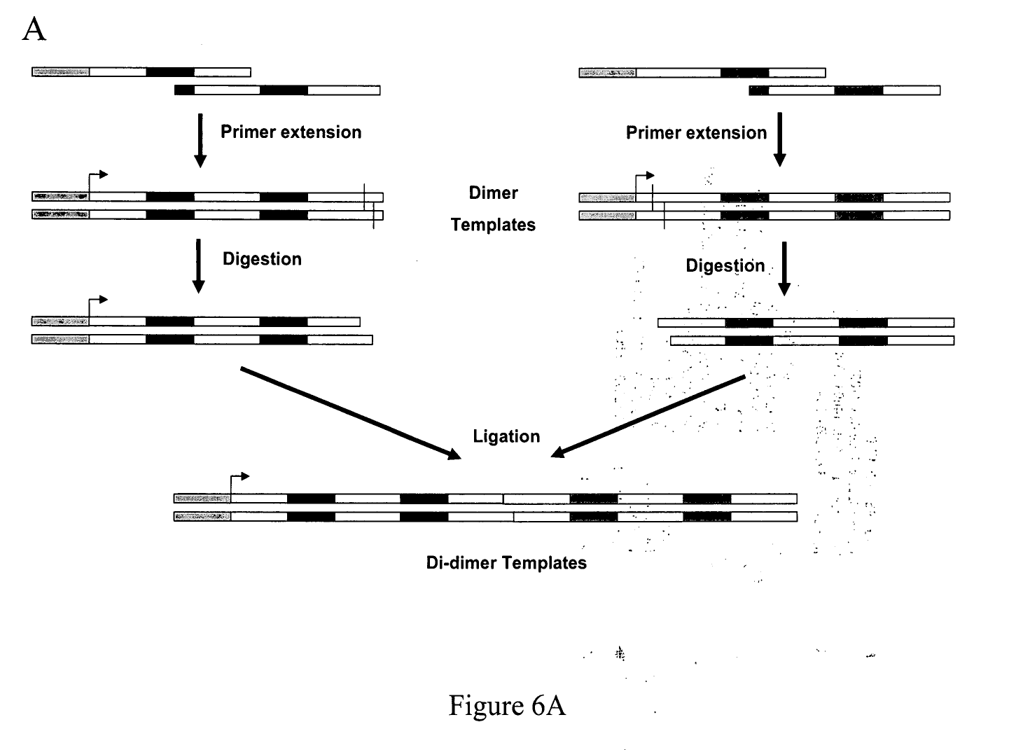

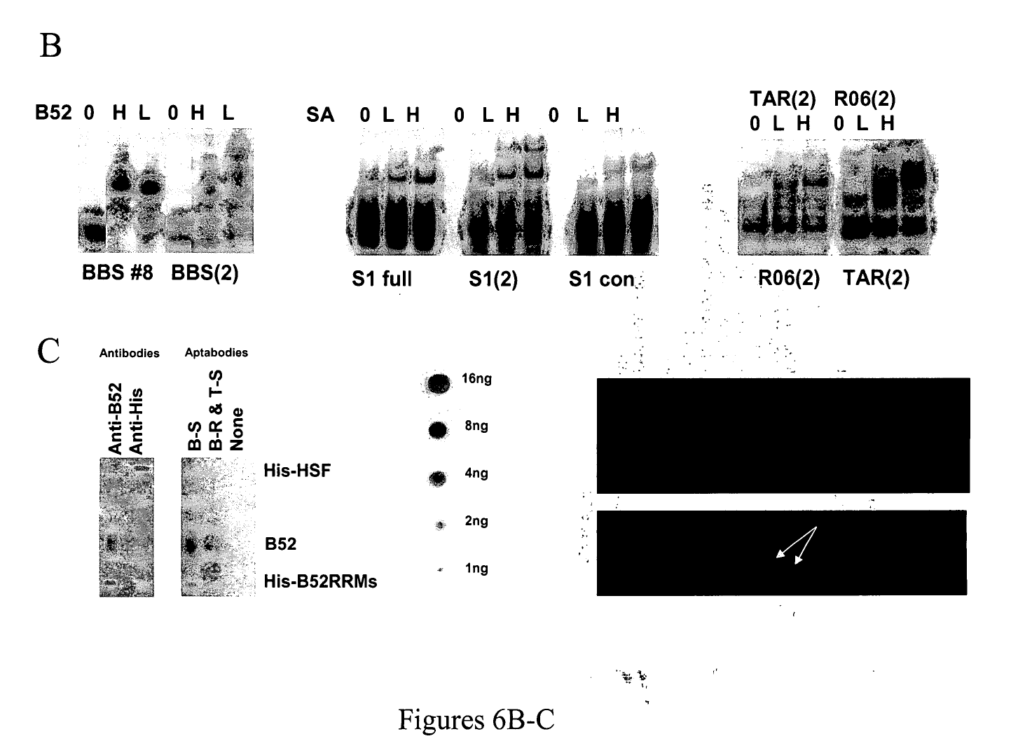

As illustrated in FIG. 6A, a pair of oligonucleotides were annealed together and underwent bi-directional primer extension under the conditions of regular PCR with only one thermo-cycle. The products were gel purified and used as templates to produce homo-dimers by in vitro transcription using the T7-MEGAscript™ in vitro transcription kit (Ambion, Inc., Austin, Tex.) according to the manufacturer's instructions. After testing the activity of the dimers by electrophoretic mobility shift (“EMS”) assay (described in Example 4 below), the templates for di-dimers were constructed by digesting the dimer templates with the restriction endonuclease BamHI, followed by ligation using T4 DNA ligase. The gel-purified products from this step served as aptabody templates in in vitro transcription using the same method described above.

Secondary structures were predicted using the folding program Mfold by Zuker and Turner, Version 3.1.

The following DNA templates are constructed to produce RNA aptabodies further described in Example 8.

Temp-BS, which has a nucleotide sequence corresponding to SEQ ID NO: 11 as follows:

| | |
|---|---|
| GTAATACGAC TCACTATAGG GATCGCCGCG GCTGGTCAAC CAGGCGACCG CCGCGGCCAC | 60 |
| AGCGGTGGGC TGGTCAACCA GGCGACCGCC CACCAGCGTT CCCGGATCCG TGACCGACCA | 120 |
| GAATCATGCA AGTGCGTAAG ATAGTCGCGG GTCGGGTCAT ACTCCCGGCC AGAATCATGC | 180 |
| AAGTGCGTAA GATAGTCGCG GGCCGGGAGG CGGATTC | 217 |

Temp-BR, which has a nucleotide sequence corresponding to SEQ ID NO: 12 as follows:

| | |
|---|---|
| GTAATACGAC TCACTATAGG GATCGCCGCG GCTGGTCAAC CAGGCGACCG CCGCGGCCAC | 60 |
| AGCGGTGGGC TGGTCAACCA GGCGACCGCC CACCAGCGTT CCCGGATCCG TGACGTCAAC | 120 |
| ACGGTCCCGG ACGTGTTGAC GTCCATACTC CTCAACACGG TCCCGGACGT GTTGAGGAGG | 180 |
| CGGATTC | 187 |

Temp-TS, which has a nucleotide sequence corresponding to SEQ ID NO: 13 as follows:

| | |
|---|---|
| GTAATACGAC TCACTATAGG GATCGCCGCC GAGCCCGGGA GCTCGGCGGC CACAGCGGTG | 60 |
| GGAGCCCGGG AGCTCCCACC AGCGTTCCCG GATCCGTGAC CGACCAGAAT CATGCAAGTG | 120 |
| CGTAAGATAG TCGCGGGTCG GGTCATACTC CCGGCCAGAA TCATGCAAGT GCGTAAGATA | 180 |
| GTCGCGGGCC GGGAGGCGGA TTC | 203 |

### Example 4

### Electrophoretic Mobility Shift Assay (EMSA)

The RNA probes were uniformly labeled with [α-³²P] UTP (Amersham Life Science Inc., Piscataway, N.J.) using the T7-MAXIscript™ in vitro transcription kit (Ambion, Austin, Tex.) according to the manufacturer's instructions. Prior to use in a binding assay, the majority of transcripts of each RNA preparation were shown to be of the expected size by electrophoresis on an 8% polyacrylamide, 7M urea gel.

All binding assays were performed in 20 μl volume. The binding buffer contains 50 mM Tris-Cl (pH 7.4), 100 mM NaCl, 15 mM MgCl₂, 10% glycerol, and 1 mM DTT. A typical binding assay using labeled RNA contains about 20 fmole of ³²P-labeled RNA probe and different amounts (1-10 pmole, indicated by L and H in FIG. 5B) of protein or non-labled RNA. The reactions were allowed to equilibrate for 15-20 minutes at ambient temperature before being subjected to EMSA.

For BBS-B52 interaction, EMS assay was performed at 4° C. The binding reaction mixtures were set at 4° C. for 5-10 minutes before being loaded onto a 2.5% agarose gel in ¼ TBE buffer. For the streptavidin aptamers and the TAR aptamers, EMSA were performed at ambient temperature with 6% or 12% native polyacrylamide gel run in TG buffer (25 mM Tris base, 200 mM glycine, and 5 mM MgCl₂).

### Example 5

### Western Blot Analysis for Antibodies and Aptabodies

Western blot analysis for antibodies was performed according to a standard protocol (Harlow et al., Using Antibodies: A Laboratory Manual. Cold Spring Harbor, Cold Spring Harbor Laboratory Press (1999), which is hereby incorporated by reference in its entirety). For the aptabodies, proteins were separated on 8% SDS-PAGE and transferred to nitrocellulose membrane according to the aforementioned standard protocol. Subsequent steps were also identical except the following substitutions. The RNA aptabody B-S (FIG. 5A) (10 μg/ml) and horseradish peroxidase conjugated streptavidin (1 μg/ml) were used to replace the primary and secondary antibodies. When the aptabodies B-R (FIG. 5B) (50 μg/ml) and T-S (FIG. 5C) (10 μg/ml) were used, they were incubated successively with the membrane, followed by incubation with horseradish peroxidase conjugated streptavidin (1 μg/ml). The blocking buffer contains 50 mM Tris (pH 7.6), 50 mM NaCl, 50 mM KCl, 15 mM MgCl₂, 1 mM PMSF, 2 mM DTT, 100 μg/ml yeast RNA, 2× Denhardt's solution, and 10% glycerol. The aptabodies were dissolved in the blocking buffer. The washing buffer contains 20 mM Tris (pH 7.6), 75 mM NaCl, 75 mM KCl, and 15 mM MgCl₂.

### Example 6

### Immunofluorescence with Antibodies and Aptabodies

Drosophila salivary glands were dissected in 0.7% NaCl and fixed in Fixing Buffer (50 μl 37% paraformaldehyde, 45011 acetic acid, 500 μl water). They were squashed on glass slides and blocked for one hour in a humid chamber with the blocking buffer described for the western blot analysis. Aptamer B-S was prepared in 200 μg/ml with 50 mM Tris (pH 7.6), 50 mM NaCl, 50 mM KCl, 15 mM MgCl₂, 100 μg/ml yeast RNA, and 1 unit/μl SUPERase-In (Ambion). 20 μl of this aptabody preparation was incubated with the squashed gland on glass slide at 4° C. overnight. Texas Red® conjugated streptavidin was prepared in 20 μg/ml in the same buffer as used for the aptabody and incubated for one hour with the squashed glands. Afterwards the slide was washed by the washing buffer described for the western blot analysis with 0.5% Tween 20 added.

### Example 7

### The Protocol of Stem Fusion: Stitching Together RNA Aptamers Through A-Form Helices and DNA Aptamers Through B-Form Helices

Aptamers are selected in a form in which the true aptamer moiety is flanked by other sequences that may not be responsible for binding to the target, and they usually have a single specificity towards the target used in the selection (Shi et al., “Evolutionary Dynamics and Population Control During In Vitro Selection and Amplification with Multiple Targets,” RNA 8:1461-1470 (2002), which is hereby incorporated by reference in its entirety). A common structural feature of many aptamers is an internal stem that appears to act as a structural anchor for recognition loops. Indeed, a library containing a stable stem-loop centered in the randomized region has been shown to be a better source of high-affinity aptamers (Davis et al., “Isolation of High-Affinity GTP Aptamers from Partially Structured RNA Libraries,” Proc. Natl. Acad. Sci. USA 99:11616-11621 (2002), which is hereby incorporated by reference in its entirety). Based on these observations, the feasibility of connecting aptamers through regular stems, which generally form A-form helices in a folded RNA structure, was explored.

Stitching together RNA modules through stems formally resembles ligation of double-strand RNA. The full-length aptamer clones isolated from the original combinatorial pool have to be reduced to functional and stably-folded secondary structure with double-stranded ends to be joined together. As shown in FIGS. 3A and 3B, for example, an RNA aptamer against Drosophila Heat Shock Factor (U.S. patent application Ser. No. 10/602,837 to Shi et al., which is hereby incorporated by reference in its entirety) and a published RNA aptamer against streptavidin (Srisawat et al., “Streptavidin Aptamers: Affinity Tags for the Study of RNAs and Ribonucleoproteins,” RNA 7:632-641 (2001), which is hereby incorporated by reference in its entirety) were used as starting material to design an RNA molecule that behaves as an antibody-mimic. These aptamers have the following sequences:

S1, which has a nucleotide sequence corresponding to SEQ ID NO: 14 as follows:

| | |
|---|---|
| GGGAGUCGAC CGACCAGAAU CAUGCAAGUG CGUAAGAUAG UCGCGGGCCG GGGGCGUAUU | 60 |
| AUGUGCGUCU ACAUCUAGAC UCAU | 84 |

RA1-HSF, which has a nucleotide sequence corresponding to SEQ ID NO: 15 as follows:

| | |
|---|---|
| GGGAGAAUUC AACUGCCAUC UAGGCAUCGC GAUACAAAAU UAAGUUGAAC GCGAGUUCUC | 60 |
| CAUCUAGUAC UACAAGCUUC UGGACUCGGU | 90 |

With the sequences of the full-length isolates, their secondary structures were predicted by energy minimization using the computer program Mfold (Zuker et al., “On Finding All Suboptimal Foldings of an RNA Molecule,” Science 244:48-52 (1989), which is hereby incorporated by reference in its entirety) as shown in FIG. 3A. Based on these structures, a series of deletion analyses were performed to reduce the aptamer to the part enclosed in the rectangular boxes. These portions retain full activity of the original aptamers and have same predicted secondary structure when folded. An example of reduced S1 has the following sequence:

S1 con, which has a nucleotide sequence corresponding to SEQ ID NO: 16 as follows:

| | |
|---|---|
| GGGACCGACC AGAAUCAUGC AAGUGCGUAA GAUAGUCGCG | 47 |
| GGCCGGG | |

The reduced HSF aptamer RA1-HSF has a three-way junction, exiting from it is a stem with an internal and an apical loop, and two other stems that can be fused with other stems. The reduced streptavidin aptamer S1 has two loops held together by a single stem. Stitching together these two reduced aptamers by stem fusion resulted in the structure shown in FIG. 3B (SEQ ID NO: 17), which is formally analogous to a monoclonal antibody against HSF as it could be used in immuno-assays, in which streptavidin is conjugated to or interacts with labeled secondary reagents. The correct folding of this composite construct is borne out by prediction through energy minimization. Since it possesses high affinity to biotin, streptavidin is used widely as an affinity tag for protein isolation and detection. Connecting the HSF aptamer to the streptavidin aptamer not only would bring the two unrelated proteins directly together, but also would indirectly connect HSF with certain enzymatic activities, fluorescent dyes, or supporting matrices like agarose beads, paramagnetic beads, quantum dots, and others. This HSF antibody-like construct, shown in FIG. 3B has the following sequence:

APTLIKE-SAHSF, which has a nucleotide sequence corresponding to SEQ ID NO: 17 as follows:

| | |
|---|---|
| GGGAUUCAAC UGCCGACCGA CCAGAAUCAU GCAAGUGCGU AAGAUAGUCG CGGGUCGGGU | 60 |
| GGCAUCGCGA UACAAAAUUA AGUUGAACGC GAGUCCCGGC CGGCCAGAAU CAUGCAAGUG | 120 |
| CGUAAGAUAG UCGCGGGCCG GCC | 143 |

This construct is divalent with regard to streptavidin and monovalent with regard to HSF. While this configuration is different from an HSF antibody, which uses two Fab fragment to interact with HSF and a single Fc fragment to interact directly or indirectly with streptavidin, it should function in a similar manner to the antibody in an immunoassay.

In the next example, a further instance of stem fusion using DNA is given. As shown in FIG. 3C, DNA is equally amenable to this manipulation. In these constructs, the DNA version of streptavidin aptamer is taken from Bittker et al., “Nucleic Acid Evolution and Minimization by Nonhomologous Random Recombination,” Nat. Biotechnol. 20:1024-1029 (2002), which is hereby incorporated by reference in its entirety. The DNA three way junction is taken from van Buuren et al., “Solution Structure of a DNA Three-way Junction Containing Two Unpaired Thymidine Bases. Identification of Sequence Features that Decide Conformer Selection,” J. Mol. Biol. 304:371-383 (2000), which is hereby incorporated by reference in its entirety, and the Taq DNA polymerase aptamer is taken from Dang et al., “DNA Inhibitors of Taq DNA Polymerase Facilitate Detection of Low Copy Number Targets by PCR,” J. Mol. Biol. 264:268-278 (1996), which is hereby incorporated by reference in its entirety. To make a DNA construct analogous to the one in FIG. 3B, the natural DNA ligand of HSF, HSE, which is described in Xiao et al., “Cooperative Binding of Drosophila Heat Shock Factor to Arrays of a Conserved 5 bp Unit,” Cell 64:585-593 (1991), which is hereby incorporated by reference in its entirety, was used. These constructs have the following sequences:

HSF-APTALIKE(DNA), which has a nucleotide sequence corresponding to SEQ ID NO: 18 as follows:

| | |
|---|---|
| AGCGGCCGTC GTTGTCGAAT GTTCTAGAAA AGCGAAAGCT TTTCTAGAAC ATTCGACCTC | 60 |
| TGTGAGACGA CGCACCGGTC GCAGGTTTTG TCTCACAGAG CGACGGCCGC TCTGTGAGAC | 120 |
| GACGCACCGG TCGCAGGTTT TGTCTCACAG | 150 |

TAQ-APTALIKE(DNA), which has a nucleotide sequence corresponding to SEQ ID NO: 19 as follows:

| | |
|---|---|
| AGCGGCCGTC GTTGTGACAA TGTACAGTAT TGGCCACCTC TGTGAGACGA CGCACCGGTC | 60 |
| GCAGGTTTTG TCTCACAGAG CGACGGCCGC TCTGTGAGAC GACGCACCGG TCGCAGGTTT | 120 |
| TGTCTCACAG | 130 |

### Example 8

### Aptabodies: Design

Although the binding sites on the Fab fragments of an antibody are presumably non-interacting and may exhibit identical changes in free energy upon binding, a divalent antibody displays a higher apparent affinity (avidity) to the antigen than isolated mono-valent Fab fragments. This phenomenon stems from the different apparent equilibrium dissociation constants for different degrees of binding site occupation on a molecule with multiple sites of same specificity. The apparent dissociation constant can be accounted for by modifying the intrinsic equilibrium dissociation constant with a “statistical factor.” To mimic this feature of enhanced avidity, two identical non-interacting sites for each aptamer targets were incorporated in a single construct, to form a “di-dimer” configuration. To avoid unnecessary complications, the two different types of aptamers in the same construct are also designed to be non-interacting.

The three-way junction layout of the RA1-HSF made it easy to be connected with two streptavidin aptamers in a design that resembles an IgG-like bivalent antibody. But many aptamers do not have more than one double-stranded end available like RA1-HSF. To present aptamers in a multivalent and multi-specificity construct, some structural elements are needed. In particular, such structural elements should have more than three double stranded “receptacles” to be connected with aptamers or other functional modules that can be made compatible with them. To this end, a dendritic scaffold that is stable and of general use was developed.

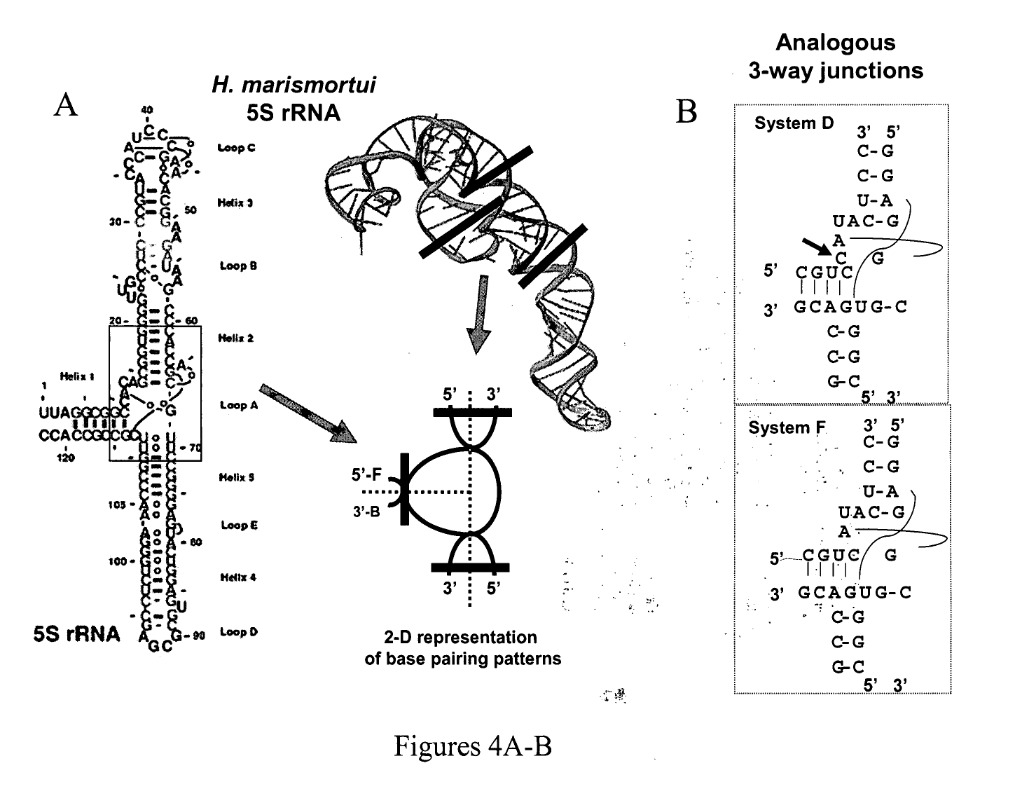

Thermodynamic and structural studies of DNA and RNA three-way junctions yielded critical insights into the factors that determine the stability of such structures. However, to maintain the stability in a construct containing more than one three-way junction, care must be taken to avoid mis-folding through unintended base pairing between parts of different three-way junctions. In other words, when fusing two three-way junctions together, they should be structurally analogous but different in sequences, especially in the stems. To fulfill these requirements, a portion of the H. marismortui 5S RNA (SEQ ID NO: 20), the structure and sequence of the Loop A and its nearby helices, were used as a first three-way junction structural element (Ban et al., “The Complete Atomic Structure of the Large Ribosomal Subunit at 2.4 A Resolution,” Science 289:905-920 (2000), which is hereby incorporated by reference in its entirety). For the second one, System D (SEQ ID NO: 21) or System F (SEQ ID NO: 22) (they vary only by one base) from a thermodynamic study was used (Diamond et al, “Thermodynamics of Three-Way Multibranch Loops in RNA,” Biochemistry 40:6971-6981 (2001), which is hereby incorporated by reference in its entirety). These have been shown to be the most stable among 12 similar structures tested. Both types of three-way junctions are depicted in FIG. 4B.

The fused double three-way junction is used to construct antibody mimics with a di-dimer arrangement, as shown in FIG. 5A. RNA constructs that have this di-dimer configuration and mimic the function of antibodies in immuno-assays are termed “aptabodies.” The aptabodies depicted in FIGS. 5A, 5B, and 5C allow the detection of the Drosophila B52 protein by streptavidin conjugates. In one aptabody construct, named B-S (FIG. 5A), two B52 aptamers (Shi et al., “A Specific RNA Hairpin Loop Structure Binds the RNA Recognition Motifs of the Drosophila SR Protein B52,” Mol. Cell. Bio. 17:1649-1657 (1997), which is hereby incorporated by reference in its entirety), and two streptavidin aptamers (Srisawat et al., “Streptavidin Aptamers: Affinity Tags for the Study of RNAs and Ribonucleoproteins,” RNA 7:632-641 (2001), which is hereby incorporated by reference in its entirety) were presented by four helices exiting from two fused three-way junctions, forming the “di-dimer” configuration. The termini of the RNA molecule form a double-strand nick with the middle portion of the sequence, which can be ligated by T4 DNA ligase to form a closed circle that is resistant to RNA exonucleases. In an immunochemical assay, this aptabody can act as the “primary antibody,” and a streptavidin conjugate can be used as the “secondary antibody.” In two other aptabodies (FIGS. 5B and 5C), the same “di-dimer” configuration is used to present two B52 aptamers and two HIV-TAR aptamers, R-06₂₄A54G (Duconge et al., “In Vitro Selection Identifies Key Determinants for Loop-Loop Interactions: RNA Aptamers Selective for the TAR RNA Element of HIV-1,” RNA 5:1605-1614 (1999), which is hereby incorporated by reference in its entirety) in one construct, and two TAR RNA elements and two streptavidin aptamers in the other. This pair of aptabodies can act respectively as the primary and secondary antibodies in an assay when a streptavidin conjugate is used as a tertiary reagent. Alternatively, the “secondary aptabody” can be labeled directly, eliminating the requirement of a tertiary reagent. A specific TAR variant, TAR-Mal, is used because it is a U-less “lipographic” site, allowing attachment of bulky fluorophores to the U-residues of the aptabody without introducing any change to the binding sites. The sequences of these three aptabodies are as follows:

B-S, which has a nucleotide sequence corresponding to SEQ ID NO: 23 as follows:

| | |
|---|---|
| GGGAUCGCCG CGGCUGGUCA ACCAGGCGAC CGCCGCGGCC ACAGCGGUGG GCUGGUCAAC | 60 |
| CAGGCGACCG CCCACCAGCG UUCCCGGAUC CGUGACCGAC CAGAAUCAUG CAAGUGCGUA | 120 |
| AGAUAGUCGC GGGUCGGGUC AUACUCCCGG CCAGAAUCAU GCAAGUGCGU AAGAUAGUCG | 180 |
| CGGGCCGGGA GGCGGAUUC | 199 |

B-R, which has a nucleotide sequence corresponding to SEQ ID NO: 24 as follows:

| | |
|---|---|
| GGGAUCGCCG CGGCUGGUCA ACCAGGCGAC CGCCGCGGCC ACAGCGGUGG GCUGGUCAAC | 60 |
| CAGGCGACCG CCCACCAGCG UUCCCGGAUC CGUGACGUCA ACACGGUCCC GGACGUGUUG | 120 |
| ACGUCCAUAC UCCUCAACAC GGUCCCGGAC GUGUUGAGGA GGCGGAUUC | 169 |

T-S, which has a nucleotide sequence corresponding to SEQ ID NO: 25 as follows:

| | |
|---|---|
| GGGAUCGCCG CCGAGCCCGG GAGCUCGGCG GCCACAGCGG UGGGAGCCCG GGAGCUCCCA | 60 |
| CCAGCGUUCC CGGAUCCGUG ACCGACCAGA AUCAUGCAAG UGCGUAAGAU AGUCGCGGGU | 120 |
| CGGGUCAUAC UCCCGGCCAG AAUCAUGCAA GUGCGUAAGA UAGUCGCGGG CCGGGAGGCG | 180 |
| GAUUC | 185 |

### Example 9

### Aptabodies: Construction and Function in Immunoassays

Use of modules simplifies modeling and verification, since they usually maintain their identity when isolated or rearranged. This example demonstrates this advantage by the process of constructing and testing the aptabodies.

The RNA aptabodies were produced by in vitro transcription from synthetic templates. The templates were constructed from fragments of oligonucleotides and contain a T7 promoter in front of the aptabody-coding region. A set of oligonucleotides was designed to produce both aptabodies and their homo-dimer components, as shown in FIG. 6A. The performance of homo-dimers of the B52 aptamer and the streptavidin aptamer were examined and compared with the corresponding monomers in either full-length or reduced forms, or both. As shown in FIG. 6B, dimers of BBS and S1 both showed an additional shifted band in EMSA whose intensity increases with increasing protein, indicating that both binding sites were active and can be occupied simultaneously. The RNA-RNA interaction between TAR and its aptamer is evident, but simultaneous occupation of two binding sites was not detected except the modest up-shift of the band representing the complex at high concentration of R-06 when TAR was labeled. With limited structural information, it is difficult to position the two binding sites in the dimers to interact at the same time.

The di-dimer aptabody templates were constructed by digestion and ligation of the templates for homo-dimers. Aptabodies were produced through in vitro transcription by the T7 RNA polymerase and their performance was compared in two immuno-assays with monoclonal antibodies. The antibodies became versatile reagents largely because they can recognize antigens presented in different environments, such as on a solid surface, in fixed tissues, or in aqueous solution, although a particular monoclonal antibody may not perform equally well under all conditions enumerated above (Harlow et al., Using Antibodies: A Laboratory Manual. Cold Spring Harbor, Cold Spring Harbor Laboratory Press (1999), which is hereby incorporated by reference in its entirety). The RNA aptamer against B52 was selected and tested by binding in aqueous solutions. For a fair comparison with antibodies, the performance of aptabodies were tested in two assay formats defined by antibodies. In both cases the target, or antigen, are presented in a non-native environment of the aptamer.

First, a Western blot analysis was conducted with the aptabodies and a monoclonal antibody against B52. The sample to be analyzed contained a full-length B52 produced from a baculovirus expression system, a His-tagged B52 deletion construct that contained the two RNA recognition motifs recognized by the aptamers and the monoclonal antibody By32 (Champlin et al., “Characterization of a Drosophila Protein Associated with Boundaries of Transcriptionally Active Chromatin,” Genes and Development 5:1611-1621 (1991), which is hereby incorporated by reference in its entirety), and a His-tagged Drosophila heat shock factor as control. These three proteins were well-separated on 8% SDS-PAGE, and commercially available monoclonal anti-His antibody was included as a second standard to be compared. The left panel of the Western blots in FIG. 6C shows the results with 10 μg/ml antibody Bv32 and anti-His antibody using 1 μg/ml of horseradish peroxidase-conjugated donkey anti-mouse IgG as secondary antibody. The right panel of the Western blots in FIG. 6C shows the results with similar amount of RNA aptabodies, using 1 μg/ml of horseradish peroxidase-conjugated streptavidin as secondary or tertiary reagent. The intensity of bands produced by both antibodies and aptabodies are comparable. The BBS recognized the target even after it had been denatured and run on SDS-PAGE and transferred to a solid phase. The methanol in the transfer buffer may have helped remove SDS from detergent-protein complex. To further measure the sensitivity of aptabodies without the complication of protein denaturing, the His-B52 RRMs construct was directly spotted onto the nitrocellulose membranes and detected as low as 1 ng of the protein with the aptabody B-S.

Next, “immunofluorescence” was performed with the aptabody B-S and Texas Red® conjugated streptavidin. Here, the target or antigen was present in fixed tissues, which usually requires 20-fold more concentrated antibody than used in Western blot analysis for detection. With similar amount of aptabody B-S and the secondary reagent, clear band patterns comparable to those generated by antibodies were observed (Champlin et al., “Characterization of a Drosophila Protein Associated with Boundaries of Transcriptionally Active Chromatin,” Genes and Development 5:1611-1621 (1991), which is hereby incorporated by reference in its entirety). These are shown in the two panels on the right of FIG. 6C. In particular, the heat shock loci are labeled intensively in samples that underwent heat treatment as shown in the lower panel, in a pattern characteristic of B52 (Champlin et al., “Characterization of a Drosophila Protein Associated with Boundaries of Transcriptionally Active Chromatin,” Genes and Development 5:1611-1621 (1991), which is hereby incorporated by reference in its entirety).

### Example 10

### Generic Scaffolds with Four Receptacles

Results described in the previous example encouraged further development of the dendritic scaffold composed of fused three-way junctions. In this example a simple method to control the three-dimensional orientations of stems exiting from the core junctions, which serve as receptacles for the functional units to be annexed to it, are described. These basic assembly units can be used to organize functional modules in space for a wide range of applications.

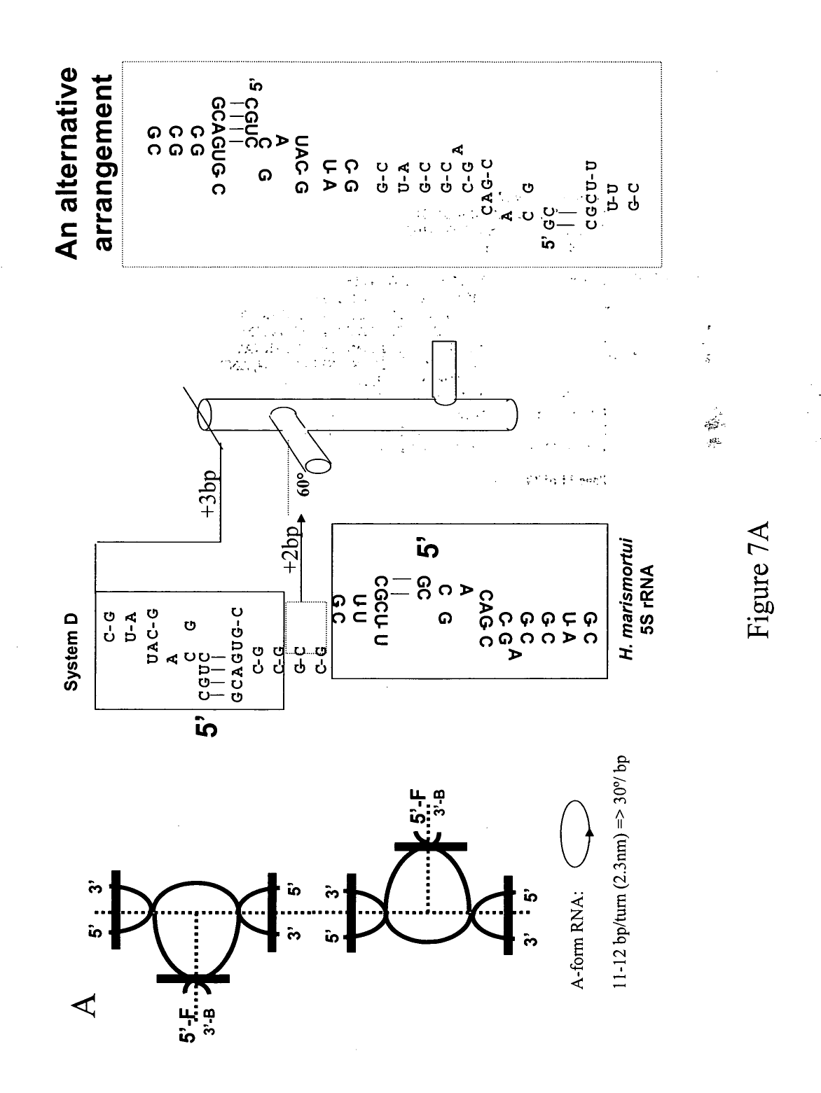

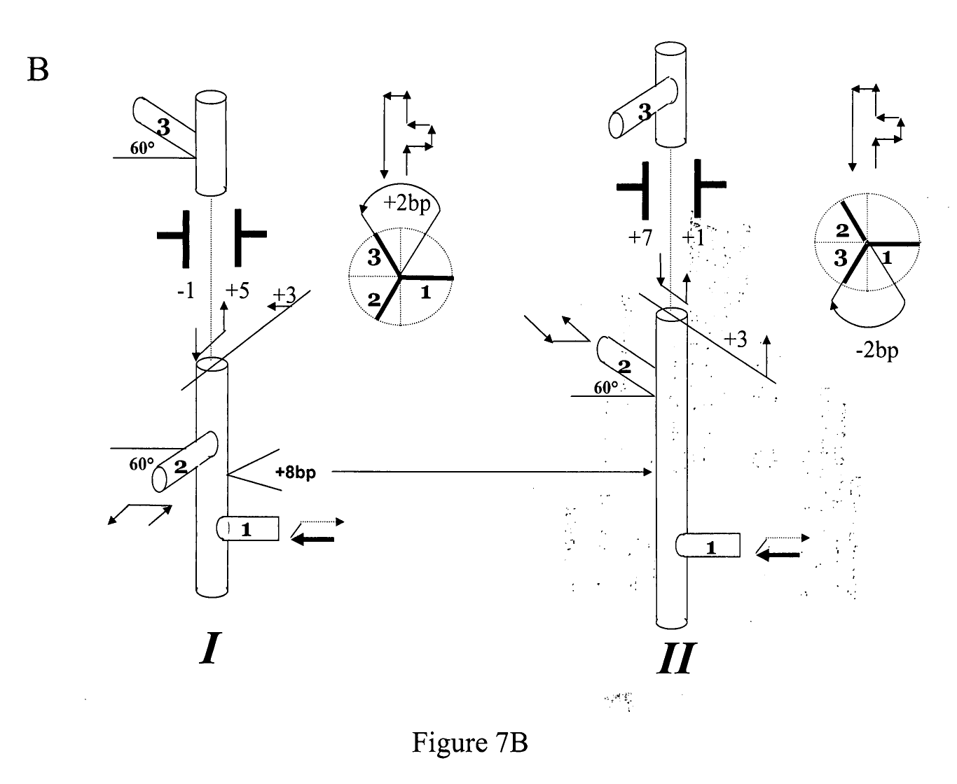

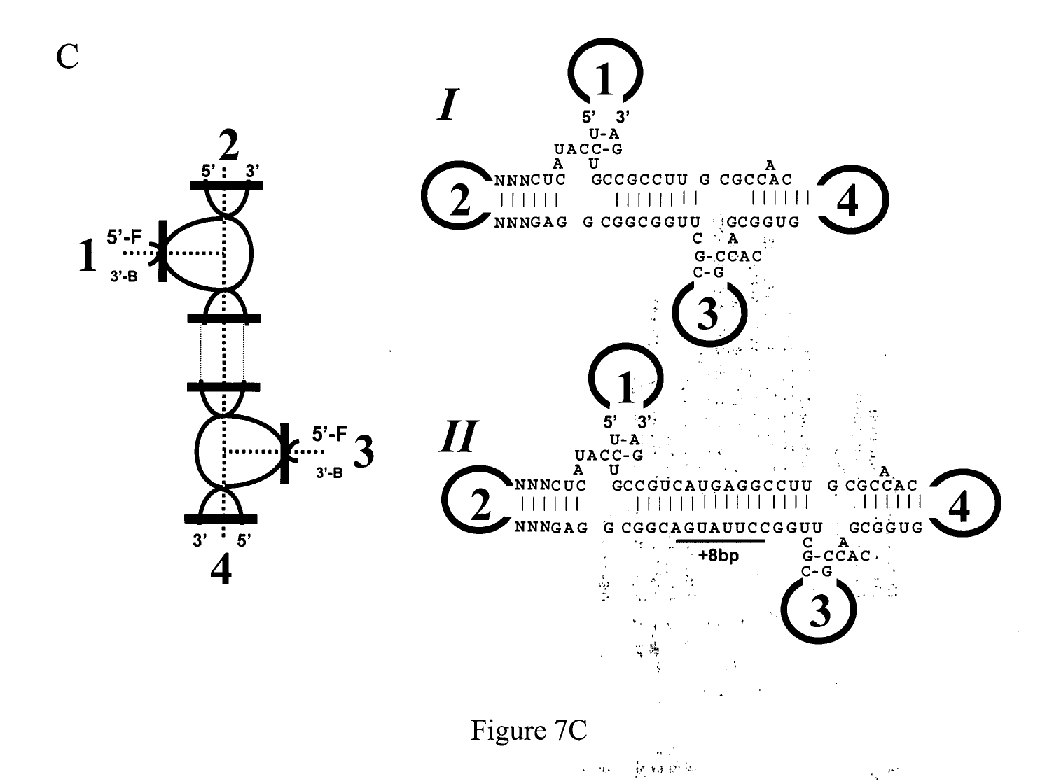

In folded RNA structures, multiple stems often stabilize one another through co-axial stacking interactions. While this is one of the structural motifs found in multibranch junctions, it is usually not considered in free-energy minimization algorithms for RNA secondary prediction. A three-way junction can have three alternatively stacked configurations. The native state is usually determined only through structural studies. Considering this situation, a three-way junction with known structure, that of the H. marismortui 5S RNA (Ban et al., “The Complete Atomic Structure of the Large Ribosomal Subunit at 2.4 A Resolution,” Science 289:905-920 (2000), which is hereby incorporated by reference in its entirety) was chosen as the first building block of the scaffold. As shown in FIG. 4A, a two-dimensional representation of stacking relationships between stems and the orientation of base pairs in the stems can be deduced from the structure. For the second three-way junction, System D or System F, which have been implied to have similar structure with the three-way junction in H. marismortui 5S RNA (Diamond et al., “Thermodynamics of Three-Way Multibranch Loops in RNA,” Biochemistry 40:6971-6981 (2001), which is hereby incorporated by reference in its entirety), were chosen. The primary sequences of these two kinds of three-way junctions are different enough to discourage the occurrence of alternative secondary structures when fused together as shown in FIG. 7A. The “tool box” of amenable structural elements like these will expand as more structural information becomes available in the public domain.

Usually RNA secondary structures are represented as two-dimensional graphs indicating the topology of binary contacts arising from specific base pairing, without referring to two- or three-dimensional geometry in terms of distance (Flamm et al., “RNA Folding at Elementary Step Resolution,” RNA 6:325-338 (2000), which is hereby incorporated by reference in its entirety). The two-dimensional representation developed here included geometric information from structural studies. It constitutes three components: a coarse grained sketch of the central loop and the exiting stems (thin curvy lines), a T-shape skeleton indicating stacking relationship (dotted lines), and base pair orientations at the stem interface (thick bars and annotations). It is simple to understand and use in the process of building more complex molecules. For example, if the two-dimensional representation is reasonably accurate, it is possible to use that information to control the orientations of stems exiting from the core in a simple and easy way, as illustrated in FIGS. 7A, 7B, and 7C. When two three-way junctions are fused together using the stacked stems, there are two alternative arrangements as depicted in FIG. 7A (SEQ ID NOS: 26, 27). One of them was chosen for further demonstration. As shown in FIG. 7B, the relative orientation of non-stacking branches 1 and 2 can be predicted and manipulated by the length of the stem that connects the two junctions. On average, each base pair turns slightly less than 30° from its nearest neighboring base pairs. Suppose one more three-way junction of known stacking arrangement is to be added to these structures, and suppose it is desirable to have the three non-stacking branches protruding from the long “trunk” in a way that minimizes the possibility of any steric hindrance, the scheme depicted in FIG. 7B can be used to aid the design. Any A-form regular stem should not be too long so as to avoid becoming a substrate for double-strand specific RNases such as Dicer (Provost et al., “Ribonuclease Activity and RNA Binding of Recombinant Human Dicer,” EMBO J. 21:5864-5874 (2002), which is hereby incorporated by reference in its entirety).

By fusing two three-way junctions, a dendritic scaffold was created with four exiting stems serving as receptacles for functional modules to be presented with favorable exposure to the solvent. The sequence of this kind of construct is formally circular with four inserts. The termini of the strand in a completed construct may be contained within any of the four inserts. The two instances depicted in FIG. 7C have the following sequences. In these two sequences, N1, N2, N3, and N4 are the sequences of the functional elements to be annexed to the four corresponding receptables to make a continuous strand transcribable from a synthetic gene. Instances of these inserts are give in Example 11.

DENDRITIC-I comprises 4 fixed sequence segments in the order of S1, S2, S3, and S4, connected by 4 undetermined sequence segments N1, N2, N3, and N4. The 5′ and 3′ ends can reside in any of these 8 segments. The fixed segments S1, S2, S3, and S4 have nucleotide sequences corresponding to SEQ ID NOs: 28-31 as set forth below.

N1, which has a nucleotide sequence corresponding to SEQ ID NO: 28 as follows:

| | |
|---|---|
| UCCAUACUC | 9 |

N2, which has a nucleotide sequence corresponding to SEQ ID NO: 29 as follows:

| | |
|---|---|
| GAGGCGGCGG UUCGC | 15 |

N3, which has a nucleotide sequence corresponding to SEQ ID NO: 30 as follows:

| | |
|---|---|
| GCCACAGCGG UG | 12 |

N4, which has a nucleotide sequence corresponding to SEQ ID NO: 31 as follows:

| | |
|---|---|
| CACCAGCGUU CCGCCGUGA | 19 |

DENDRITIC-II comprises 4 fixed sequence segments in the order of S1, S2, S3, and S4, connected by 4 undetermined sequence segments N1, N2, N3, and N4. The 5′ and 3′ ends can reside in any of these 8 segments. The fixed segments S1, S2, S3, and S4 have nucleotide sequences corresponding to SEQ ID NOs: 32-35 set forth below.

N1, which has a nucleotide sequence corresponding to SEQ ID NO: 32 as follows:

| | |
|---|---|
| UCCAUACUC | 9 |

N2, which has a nucleotide sequence corresponding to SEQ ID NO: 33 as follows:

| | |
|---|---|
| GAGGCGGCAG UAUUCCGGUU CGC | 23 |

N3, which has a nucleotide sequence corresponding to SEQ ID NO: 34 as follows:

| | |
|---|---|
| GCCACAGCGG UG | 12 |

N4, which has a nucleotide sequence corresponding to SEQ ID NO: 35 as follows:

| | |
|---|---|
| CACCAGCGUU CCGGAGUACU GCCGUGA | 27 |

Secondary structure prediction based on these sequences without inserts using Mfold yielded structures as anticipated. Considering additional free energy contributions from non-canonic base pairing, stacking, and other factors, these structures should form and be stable under normal in vitro and in vivo conditions.

### Example 11

### A General Scheme for Presenting RNA Aptamers In Vivo

Any RNA aptamer or non-aptamer functional elements with at least one double-stranded end can be used as a module and presented from any receptacle of the scaffold described in the previous example. Use of modules facilitates independent variation and evolvability of components and sub-systems. And the aptabodies described in Examples 8 and 9 provided an experimentally validated case, which demonstrated that RNA modules do maintain their identity whether isolated or incorporated in a composite structure with this kind of scaffold. The general utility of the generic scaffolds is now demonstrated by combinatorial annexation of aptamer as well as non-aptamer functional modules to them. These functional modules act as basic assembly units for constructing complex RNA molecules in a manner like synthons used in organic chemistry. The method described here also illustrates induced proximity via protein-like RNA constructs.

For illustration, consider the problem of efficient expression of RNA aptamers in vivo. Here one aptamer constitutes the “essential” activity of the construct. A few “non-essential” ancillary elements are included to enhance the robustness of the “essential” activity. For considerations discussed later Receptacle 2 of the dendritic scaffold was chosen as the aptamer receptacle and the other three as “service receptacles” for ancillary functions such as stability, transportation, localization, oligomerization, etc. The sequence inserts are N1 through N4 as mentioned in the previous example. The modules to be grafted to Receptacle 1 through 4 have functions designated as follows: N1—accumulation and stability; N2—aptamer presentation; N3—ligomerization; and N4—transportation and localization.

In addition to their capability to be engrafted to the dendritic scaffolds through stem fusion, all functional modules described below are genetically, biochemically, and structurally well-characterized. Moreover, their modality has been demonstrated in multiple different sequence and structural contexts. Like the modules used to construct aptabodies, it is reasonable to expect them to maintain their identity in the composite constructs.

When a functional RNA element, including aptamer, is to be delivered into cells or organisms as a synthetic gene, two of the first issues to be considered are accumulation and stability. Previously, a scheme was developed to address both, which can be easily incorporated into the current method. To increase the accumulation, one might increase the rate of production and decrease the rate of degradation (U.S. Pat. No. 6,458,559 to Shi et al., which is hereby incorporated by reference in its entirety). To increase the rate of production, polymers of the RNA coding unit were made in the synthetic gene to increase the number of aptamers transcribed following a single transcription initiation event. The multimeric transcript was then divided into the intended units by self-cleavage reaction of a cis-acting hammerhead ribozyme included in each unit. After cleavage, a new hammerhead ribozyme was formed for each unit through base pairing of regions near the 5′ and 3′ termini of the unit. Since hammerhead ribozyme, like a few other small ribozymes, cleaves an RNA phosphodiester backbone to yield a 5′ hydroxyl and a 2′, 3′ cyclic phosphodiester as products (Buzayan et al., “Autolytic Processing of a Phosphorothioate Diester Bond,” Nucleic Acids Res. 16:4009-4023 (1988), which is hereby incorporated by reference in its entirety), it can also ligate the products using the substrate bond energy conserved in the non-hydrolyzed cyclic product to re-form a phosphodiester. In the present construct the “enzyme moiety” and the “substrate moiety” of the ribozyme are covalently linked together through the rest of the RNA molecule and cannot diffuse apart, thus it is expected that the construct will spend considerable time in a covalent circular form resistant to exonuclease digestion. Should this first line of resistance fail, the S35 motif next to the catalytic core should resist single-strand specific RNases and enhance the stability of the molecule as demonstrated before (Thompson et al., “Improved Accumulation and Activity of Ribozymes Expressed from a tRNA-Based RNA Polymerase III Promoter,” Nucleic Acid Res. 23:2259-2268 (1995), which is hereby incorporated by reference in its entirety). This, too, is described in U.S. Pat. No. 6,458,559 to Shi et al., which is hereby incorporated by reference in its entirety.

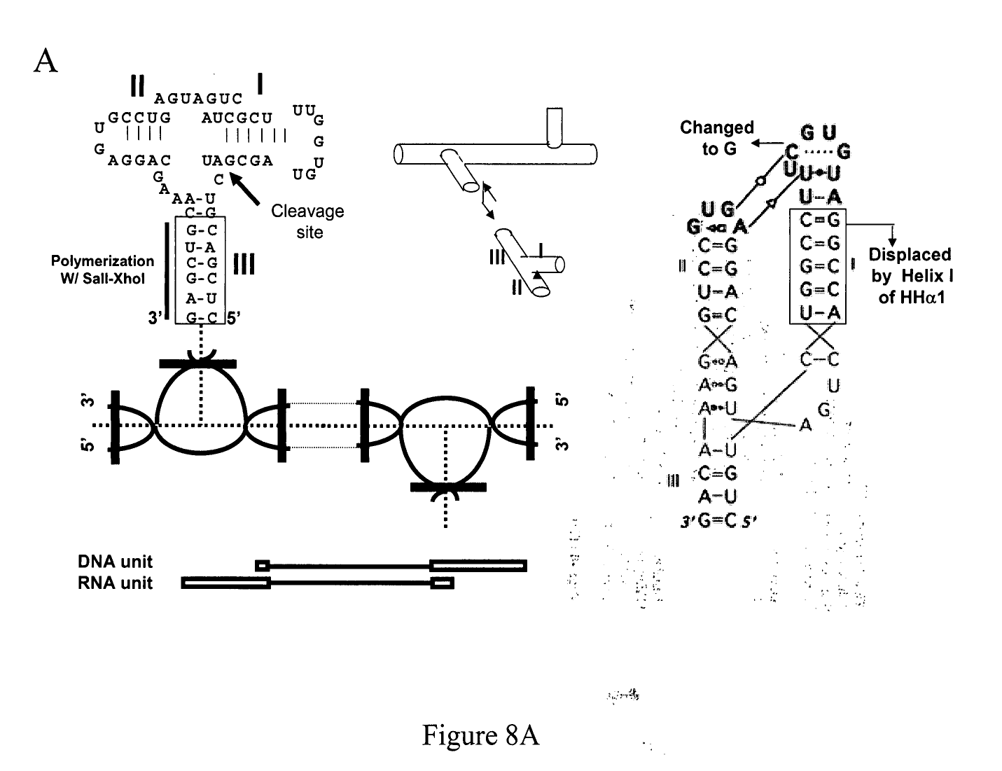

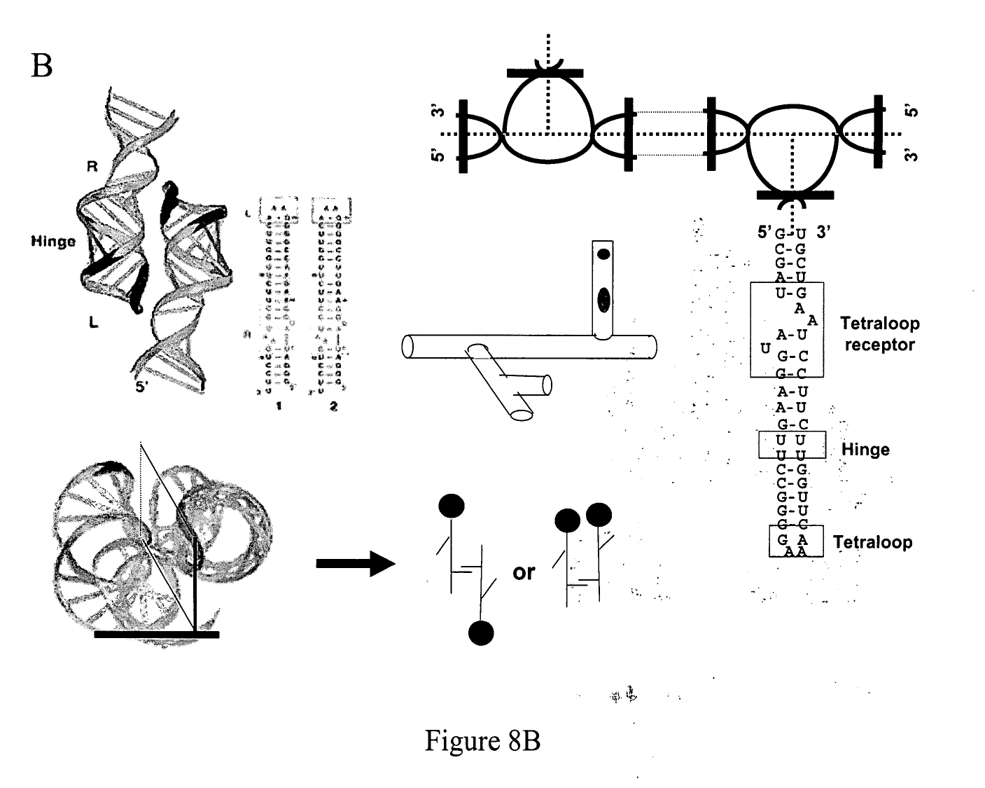

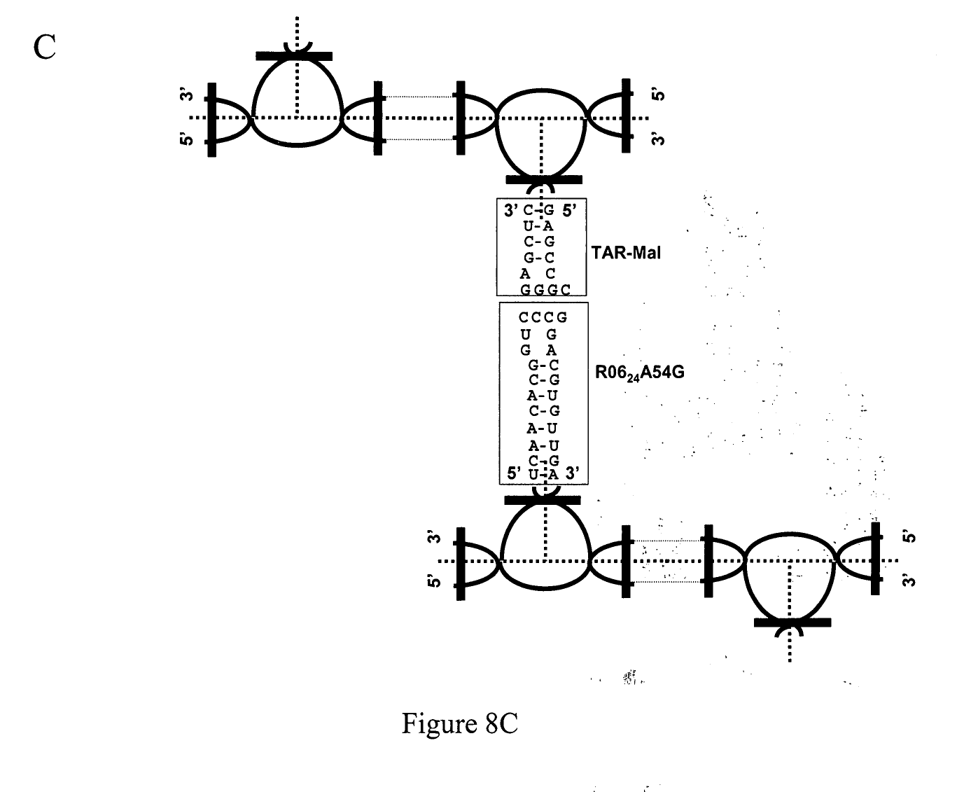

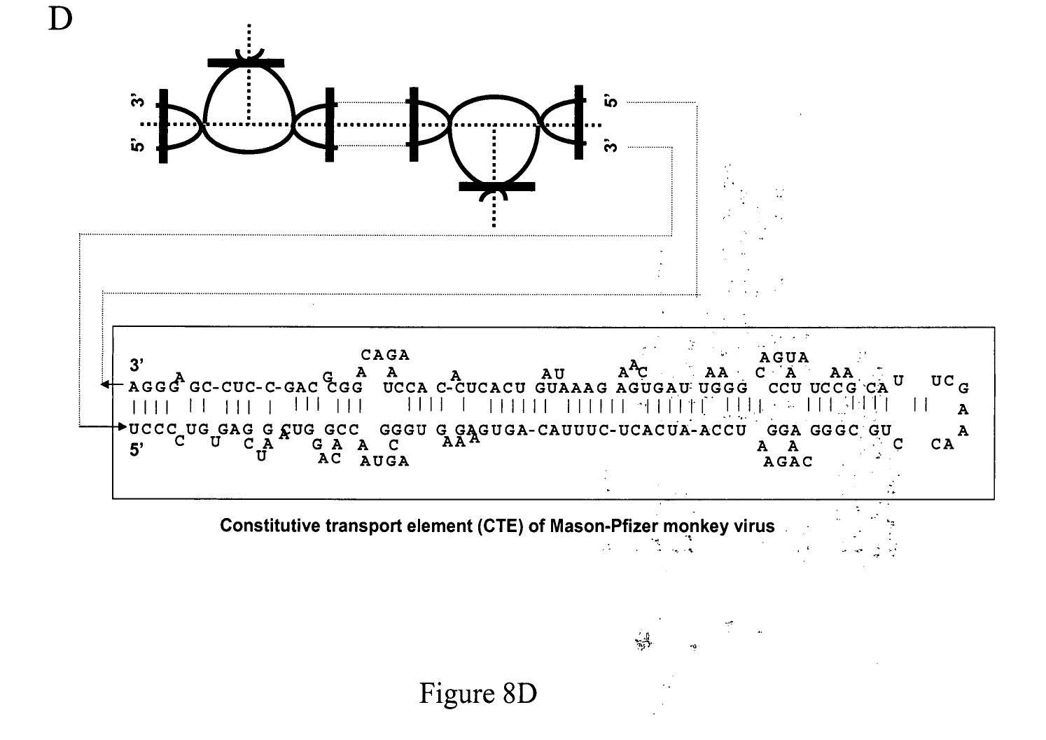

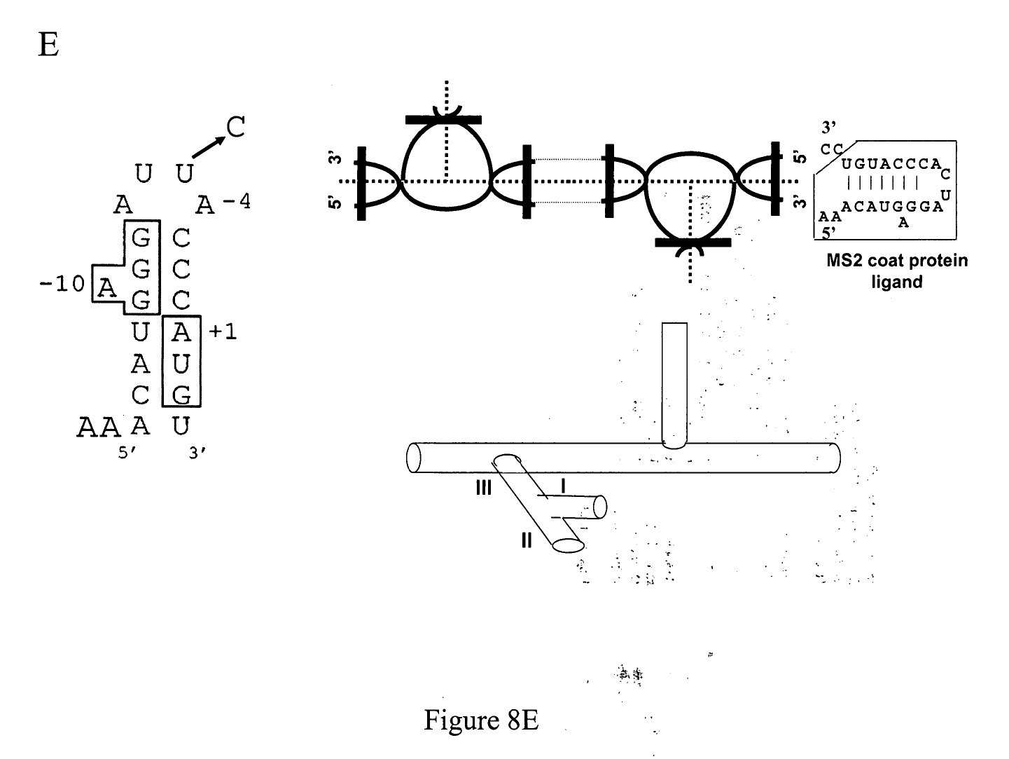

A new hammerhead ribozyme was designed and engrafted to Receptacle 1 of the dendritic scaffold. The backbone sequence and stem-loop configuration are based on the hammerhead motif of satellite RNA of tobacco ringspot virus (sTobRV) (Buzayan et al., “Autolytic Processing of a Phosphorothioate Diester Bond,” Nucleic Acid Res. 16:4009-4023 (1988), which is hereby incorporated by reference in is entirety). As shown in FIG. 8A, two changes were made to improve the cleavage rate inside cells. First, the C residue at position L1.5 was changed to a G (Khvorova et al., “Sequence Elements Outside the Hammerhead Ribozyme Catalytic Core Enable Intracellular Activity,” Nature Structural Biology 10:708-712 (2003), which is hereby incorporated by reference in its entirety). Second, Stem I was replaced by that of HHα1 (Clouet-d'Orval et al., “Hammerhead Ribozymes with a Faster Cleavage Rate,” Biochemistry 36:9087-9092 (1997), which is hereby incorporated by reference in its entirety). To facilitate polymerization and cloning, a SalI/XhoI fusion site was incorporated into the 3′ side of Stem III. This arrangement also ensures a S35 motif would form for the first unit in a polymer. The hammerhead ribozyme itself has a three-way junction arrangement in which Stem II and III stack with each other and form a sharp angle with Stem I, resulting in a wishbone-like structure. Stem III was elongated and connected to Receptacle 1 of the scaffold. The length of Stem III gives a rough indication of the orientation of Stem I in the construct.

As mentioned above, the sequence of the generic scaffold is formally circular, and the hammerhead ribozyme will determine the termini of the construct. Eukaryotic transcription proceeds at a rate of 20-30 bases per second, thus secondary structures will form co-transcriptionally. The ribozyme cleavage reaction was expected to occur in less than a minute (Clouet-d'Orval et al., “Hammerhead Ribozymes with a Faster Cleavage Rate,” Biochemistry 36:9087-9092, (1997), which is hereby incorporated by referent in its entirety). Previously, this enzyme was used to cleave a homopolymer of pentavalent aptamers. See U.S. Pat. No. 6,458,559 to Shi et al., which is hereby incorporated by reference in its entirety. Notably, different units can also be connected in a single transcript and cleaved by identical ribozymes as demonstrated before. The sequence of the hammerhead ribozyme described above, which constitutes N1 in “SEQ Dendritic Scaffold I and II”, is as follows:

N1 (HH), which has a nucleotide sequence corresponding to SEQ ID NO: 36 as follows:

| | |
|---|---|
| CUCGACGUCU AGCGAUGUGG UUUCGCUACU GAUGAGUCCG UGAGGACGAA ACGUCGAG | 58 |

Proteins often assemble into large structures. This strategy of using non-covalent binding of smaller subunits to build larger structures is also advantageous if adopted in RNA molecular design. Here Receptacle 3 of the scaffold is dedicated to the function of nano-scale self-assembly through tertiary interaction between synthetic modular RNA units. A method to make homodimers or heterodimers of the RNA constructs is also described.

To induce dimerization of two identical RNA constructs, a published tectoRNA module (Jaeger et al., “TectoRNA: Modular Assembly Units for the Construction of RNA Nano-Objects,” Nucleic Acids Res. 29:455-463 (2001), which is hereby incorporated by reference in its entirety) can be directly connected to Receptacle 3. This module has a stem with an apical tetraloop and a tetraloop-receptor incorporated in the middle. Its design was based on the crystal structure of the tertiary interactions formed by the GAAA tetraloop and its “11-nucleotide receptor” motif. Dimerization of the smallest tectoRNA molecules 1 or 2 as shown in FIG. 8B occurs with a Kd of 4.3 nM at 15 mM magnesium ions. Since the loop and its receptor are in phase on one side of the helix, it is possible to control the relative position of the aptamer moiety attached to Receptacle 2 by the length of the stem between the loop receptor and the three-way junction from which this module protrudes, as illustrated in FIG. 8B. The sequence of the module to be incorporated, N3-1, is as follows:

N3-1 (LR), which has a nucleotide sequence corresponding to SEQ ID NO: 37 as follows:

| | |
|---|---|
| GCGAUAUGGA AGUUCCGGGG AAACUUGGUU CUUCCUAAGU CGU | 43 |

The interaction between the tetraloop and its receptor is asymmetric, like that between a male and a female socket. The tectoRNA module converted an asymmetric interaction into a symmetric interaction with increased stability to create homodimers. A single pair of tetraloop and its receptor may be used on different molecules to form heterodimers with less stability. Other RNA-RNA interacting modules also exist and can be used for this purpose. In Examples 8 and 9, HIV TAR module and its aptamer were used to form heterodimers that withstood long and extensive washing. This pair can also be used as depicted in FIG. 8C, with the following N3 sequences:

N3-2A (TAR-MAL), which has a nucleotide sequence corresponding to SEQ ID NO: 38 as follows:

| | |
|---|---|
| GAGCCCGGGA GCUC | 14 |

N3-2B (R-06₂₄A54G), which has a nucleotide sequence corresponding to SEQ ID NO: 39 as follows:

| | |
|---|---|
| UCAACACGGU CCCGGACGUG UUGA | 24 |

Uncapped and without polyadenylation signal, the ribozyme-cleaved constructs will be retained inside the nuclei. While this default setting is beneficial when the aptamer is intended to function in nuclei, similar selective localization in other subcellular compartments is also desirable. To fulfill this requirement, Receptacle 4 is dedicated to the function of active transportation and localization, which can be illustrated with two methods.

First, consider active transportation out of nuclei into cytoplasm. Here it is proposed to use a structured retroviral RNA element that mediates nucleocytoplasmic export of intron-containing RNA. This constitutive transport element (CTE) from the Mason-Pfizer monkey virus (MPMV), it is shown, can be easily incorporated into the dendritic scaffold. To export unspliced viral genome for packaging into progeny particles, type D retroviruses such as MPMV evolved CTE (Ernst et al., “A Structured Retroviral RNA Element that Mediates Nucleocytoplasmic Export of Intron-Containing RNA,” Mol. Cell Biol. 17:135-144 (1997), which is hereby incorporated by reference in its entirety), directly bind to a cellular mRNA export factor, the mammalian TAP (NXF1) protein (Braun et al., “TAP Binds to the Constitutive Transport Element (CTE) Through a Novel RNA-Binding Motif that is Sufficient to Promote CTE-Dependent RNA Export from the Nucleus,” Embo. J. 18:1953-1965 (1999), which is hereby incorporated by reference in its entirety). The CTE forms a long stem containing an apical loop and a mirror-symmetric pair of internal loops (Ernst et al., “Secondary Structure and Mutational Analysis of the Mason-Pfizer Monkey Virus RNA Constitutive Transport Element,” RNA 3:210-222 (1997), which is hereby incorporated by reference in its entirety). Genomic SELEX using the human genome uncovered a group of TAP-binding elements that are homologous to the core TAP-binding sites in CTE (Zolotukhin et al., “Retroviral Constitutive Transport Element Evolved from Cellular TAP(NXF1)-Binding Sequences,” J. Virol. 75:5567-5575 (2001), which is hereby incorporated by reference in its entirety). As shown above with other elements that form a stem, CTE can be annexed to Receptacle 4 without difficulty, as shown in FIG. 8D. The sequence to be inserted as N4 is:

N4-1 (MPMVCTE8009-8170), which has a nucleotide sequence corresponding to SEQ ID NO: 40 as follows:

| | |
|---|---|
| UCCCCUGUGA GCUAGACUGG ACAGCCAAUG ACGGGUAAGA GAGUGACAUU UCUCACUAAC | 60 |
| CUAAGACAGG AGGGCCGUCA AAGCUACUGC CUAAUCCAAU GACGGGUAAU AGUGACAAGA | 120 |
| AAUGUAUCAC UCCAACCUAA GACAGGCGCA GCCUCCGAGG GA | 162 |

The second instance of Receptacle 4 usage is to further localize the aptamer to a sub-nuclear location, a particular promoter of gene. RNA can be tethered to DNA in sequence-specific manner through a protein adapter that binds both DNA and RNA specifically. In particular, a LexA-MS2 coat protein fusion construct can bring RNA to the promoter of a LexA-regulated gene, and a GAL4 DNA binding domain-MS2 coat protein fusion construct can bring RNA to the promoter of a GAL4-regulated gene, if the RNA bears an MS2 coat protein ligand. Yeast strains harboring this kind of reporter gene and expressing these fusion proteins are available easily and commercially as parts of a “three-hybrid” system for detecting RNA-protein interaction in vivo (U.S. Pat. Nos. 5,610,015; 5,677,131; and 5,750,667 to Wickens et al., each of which is hereby incorporated by reference in its entirety). The coat protein of bacteriophage MS2, like the nearly identical protein from bacteriophage R17, recognizes a 21-base RNA stem-loop in its genome with high affinity. This functional module can be easily annexed to our dendritic scaffold, as shown in FIG. 8E. The following sequence, N4-2, is based on the results of an in vitro selection experiment with the R17 coat protein (Schneider et al., “Selection of High Affinity RNA Ligands to the Bacteriophage R17 Coat Protein,” J. Mol. Biol. 228:862-869 (1992), which is hereby incorporated by reference in its entirety).

N4-2 (MS2SELECT), which has a nucleotide sequence corresponding to SEQ ID NO: 41 as follows:

| | |
|---|---|
| AAACAUGAGG AUCACCCAUG U | 21 |

### Example 12

### A Specific Construct Designed to Bring Proteins to a Promoter

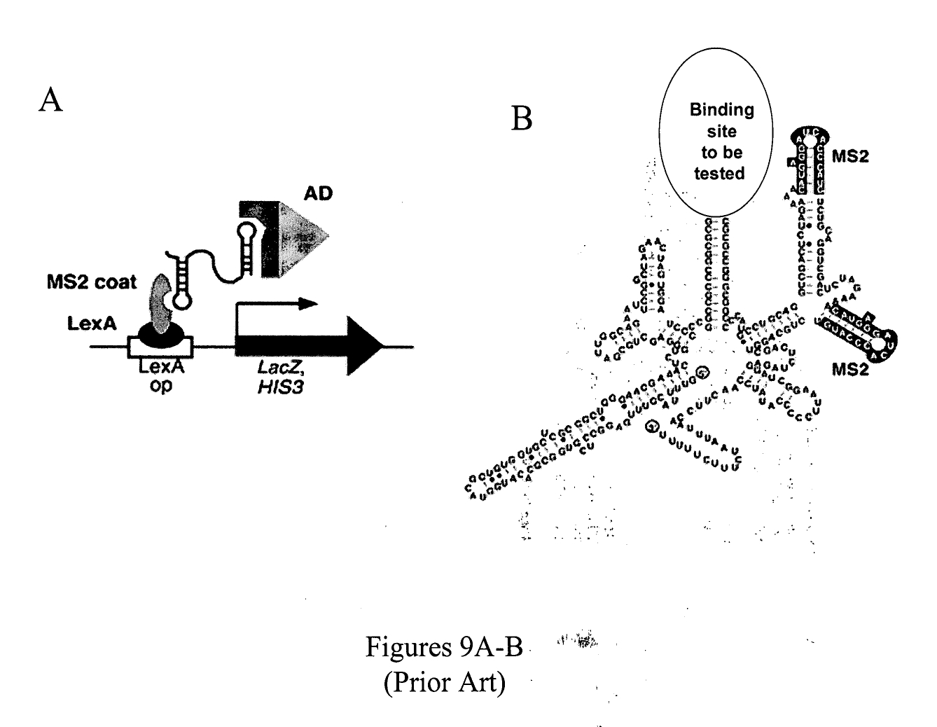

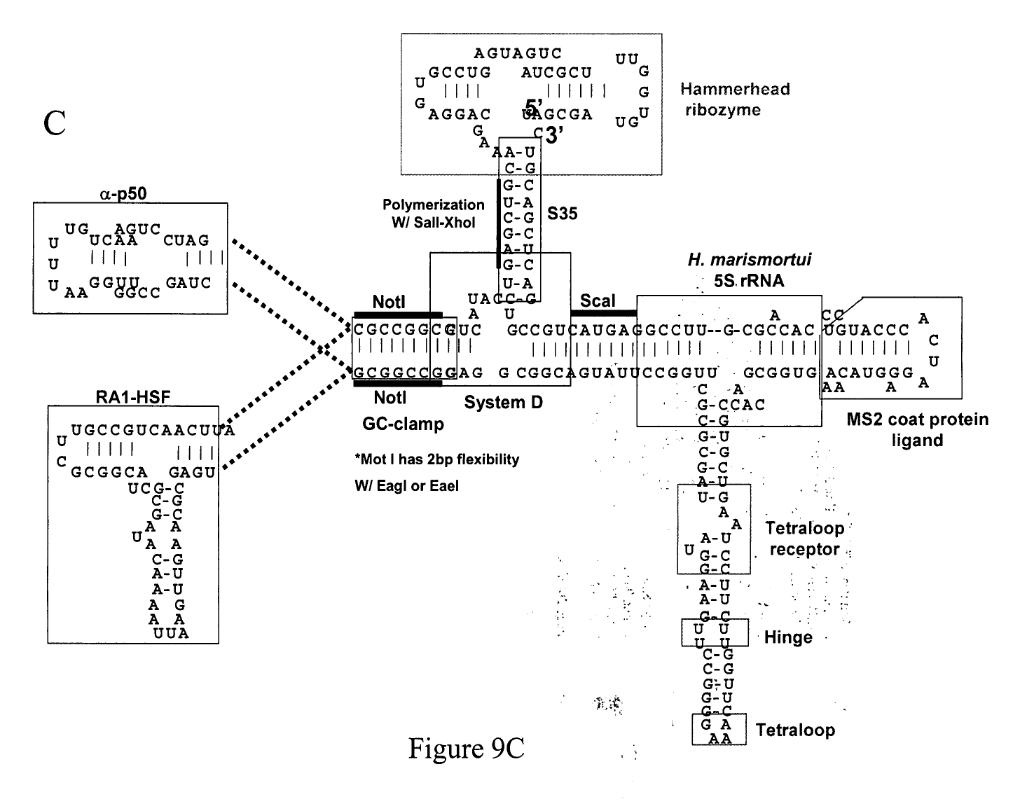

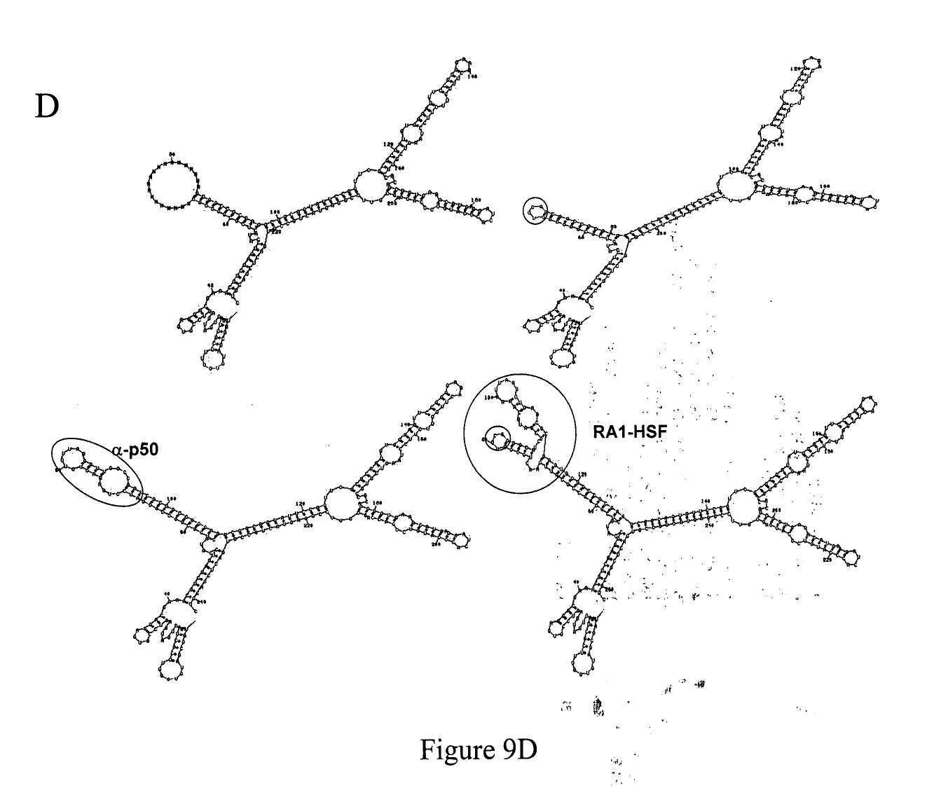

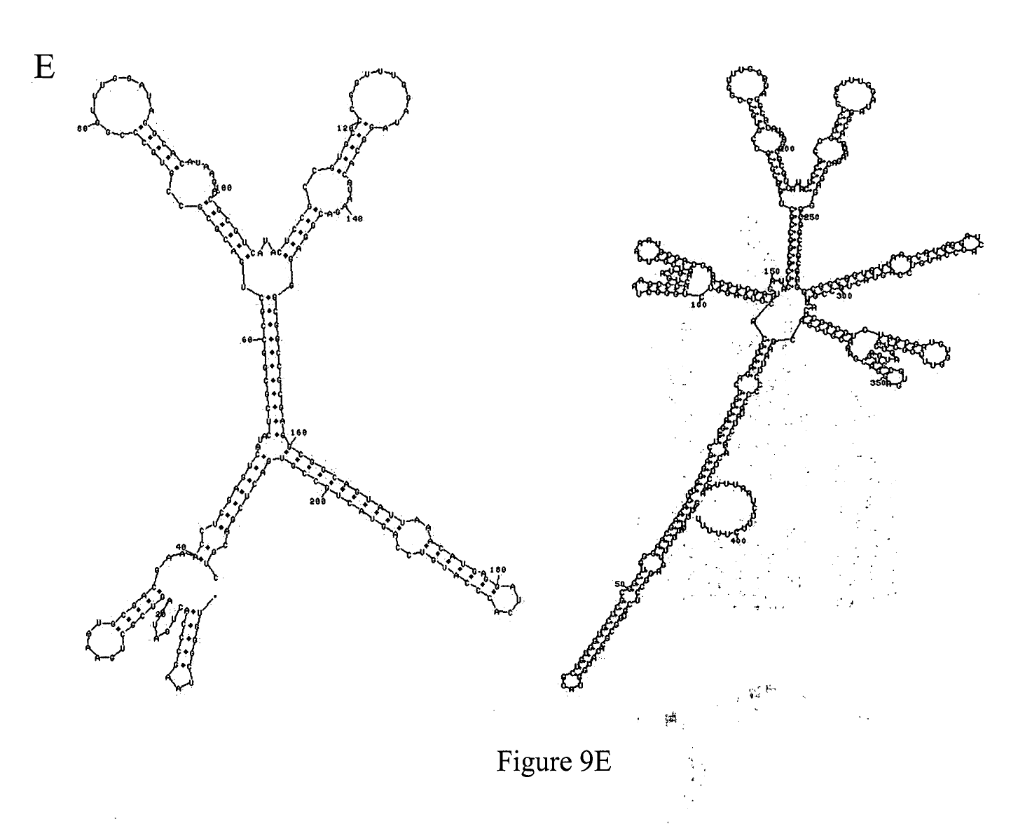

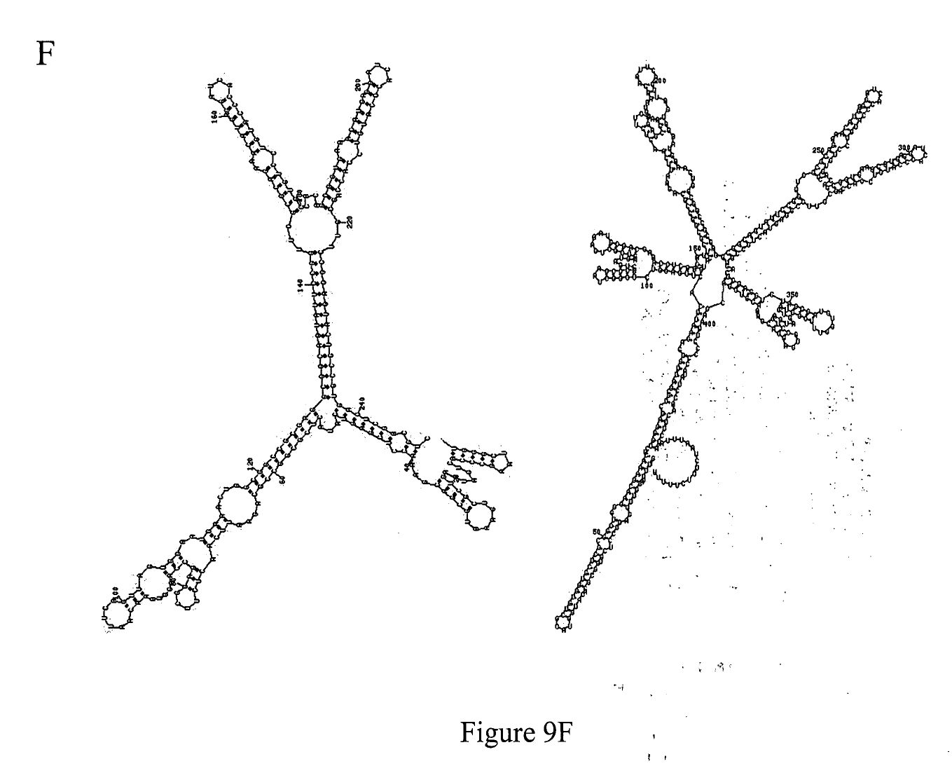

With the modules enumerated in the previous two examples, a process of assembly is described to make a single construct using the protocol of stem fusion. As mentioned above, a yeast “three-hybrid” system, depicted in FIG. 9A, was developed to detect RNA-protein interaction in vivo (U.S. Pat. Nos. 5,610,015, 5,677,131, and 5,750,667 to Wickens et al., which are hereby incorporated by reference in its entirety). It was also used to optimize an RNA aptamer (Cassiday et al., “Yeast Genetic Selections to Optimize RNA Decoys for Transcription Factor NF-kappa B,” Proc. Natl. Acad. Sci. USA 100:3930-3935 (2003), which is hereby incorporated by reference in its entirety). In these genetic assays, RNA-protein interactions are detected in a fashion independent of the biological function of the RNA or protein. Here, a new use of this system is proposed to study the effects of aptamers, especially those against transcription factors, on transcription. In particular, a construct composed of modules mentioned above, which substitutes the “RNA hybrid” depicted in FIG. 9B, and carries additional features is described. This will allow one to utilize the promoter of existing reporter genes and fusion DNA/RNA-binding proteins to recruit aptamers to specific genes.

In this construct, a hammerhead ribozyme (SEQ N1), a tectoRNA module (N-3-1), and a MS2 coat protein ligand (N-4-2) are selected from the collection of functional modules enumerated in the previous example and engrafted to Receptacles 1, 3, and 4, respectively, of the dendritic scaffold II. For the purpose of illustration, the HSF aptamer described in Example 1 or a published NF-κB aptamer (Lebruska et al., “Selection and Characterization of an RNA Decoy for Transcription Factor NF-kappa B,” Biochemistry 38:3168-3174 (1999), which is hereby incorporated by reference in its entirety) was arbitrarily chosen as the aptamer to be recruited to the promoter. In these cases, the induced proximity of the transcription factors and MS2 coat protein could bring HSF or NF-κB to the promoter of a non-heat responding and non-NF-κB activated gene, respectively. The effect may be studied under different conditions, such as both heat shock and non-heat shock conditions for HSF. The non-aptamer part of the DNA construct is designed to be embedded in a cloning vector, so the aptamer-encoding sequence can be engrafted to Receptacle 2 in a regular sub-cloning process through a Not I site on the vector. Not I recognizes an 8-base pair DNA sequence solely composed of CG bases. This sequence in the RNA construct serves as a “GC-clamp” that stabilizes the stem and insulates the incoming aptamer from the rest of the construct to reduce the occurrence of alternative secondary structures. The length of this clamp can be shortened by one or two base pairs if the insert is prepared by EagI or EaeI digestion. The coding sequence of the DNA construct with aptamer inserted can be polymerized through the isoschismer sites of SalI and XhoI as described in U.S. Pat. No. 6,458,559 to Shi et al., which is hereby incorporated by reference in its entirety. The linear coding sequence in the vector starts at the SalI site and ends at the XhoI site, both are to be destroyed in a polymer to ensure “head-to-tail” ligation. A ScaI site is incorporated in the middle of the sequence to assist the characterization of cloning products.

The resulting RNA sequence was checked by submitting it to the RNA folding program Mfold. FIG. 9D shows the predicted secondary structures of constructs (by Mfold) with different sequences inserted into the cloning site. The RNA construct without aptamer (FIG. 9D, upper left panel) has the following sequence (NN signifies any aptamer insert):

RNAHYB-VEC, which has a nucleotide sequence corresponding to SEQ ID NO: 42 as follows:

| | |
|---|---|
| UAGCGAUGUG GUUUCGCUAC UGAUGAGUCC GUGAGGACGA AACGUCGAGU CCAUACUCGC | 60 |
| GGCCGCNNGC GGCCGCGAGG CGGCAGUAUU CCGGUUCGCG CGAUAUGGAA GUUCCGGGGA | 120 |
| AACUUGGUUC UUCCUAAGUC GUGCCACAGC GGUGAAACAU GAGGAUCACC CAUGUCCCAC | 180 |
| CAGCGUUCCG GAGUACUGCC GUGACUCGAC GUC | 213 |

An alternative construct without aptamer (FIG. 9D, upper right panel) has a nucleotide sequence corresponding to SEQ ID NO: 54 as follows:

UAGCGAUGUG GUUUCGCUAC UGAUGAGUCC GUGAGGACGA AACGUCGAGU CCAUACUCGC
GGCCGCUUCG GCGGCCGCGA GGCGGCAGUA UUCCGGUUCG CGCGAUAUGG AAGUUCCGGG
GAAACUUGGU UCUUCCUAAG UCGUGCCACA GCGGUGAAAC AUGAGGAUCA CCCAUGUCCC
ACCAGCGUUC CGGAGUACUG CCGUGACUCG ACGUC

Insertion of the HSF aptamer RA1-HSF or the NF-κB aptamer αp50 results in the following sequences:

RNAHYB-RA1 (FIG. 9D, lower right panel), which has a nucleotide sequence corresponding to SEQ ID NO: 43 as follows:

| | |
|---|---|
| UAGCGAUGUG GUUUCGCUAC UGAUGAGUCC GUGAGGACGA AACGUCGAGU CCAUACUCGC | 60 |
| GGCCGCAUUC AACUGCCGUU CGCGGCAUCG CGAUACAAAA UUAAGUUGAA CGCGAGUGCG | 120 |
| GCCGCGAGGC GGCAGUAUUC CGGUUCGCGC GAUAUGGAAG UUCCGGGGAA ACUUGGUUCU | 180 |
| UCCUAAGUCG UGCCACAGCG GUGAAACAUG AGGAUCACCC AUGUCCCACC AGCGUUCCGG | 240 |
| AGUACUGCCG UGACUCGACG UC | 262 |

RNAHYB-NF (FIG. 9D, lower left panel), which has a nucleotide sequence corresponding to SEQ ID NO: 44 as follows:

| | |
|---|---|
| UAGCGAUGUG GUUUCGCUAC UGAUGAGUCC GUGAGGACGA AACGUCGAGU CCAUACUCGC | 60 |
| GGCCGCGAUC CUGAAACUGU UUUAAGGUUG GCCGAUCGCG GCCGCGAGGC GGCAGUAUUC | 120 |
| CGGUUCGCGC GAUAUGGAAG UUCCGGGGAA ACUUGGUUCU UCCUAAGUCG UGCCACAGCG | 180 |
| GUGAAACAUG AGGAUCACCC AUGUCCCACC AGCGUUCCGG AGUACUGCCG UGACUCGACG | 240 |
| UC | 242 |

In addition to bringing proteins to a promoter, this scheme has other utilities. In particular, when the target of the aptamers being presented in this construct is a transcription factor recruited to the promoter during transcription initiation, reinitiation, or both, and is required for the functioning of the promoter, this construct can function as a probe to study these factors' involvement in the process of transcription initiation, reinitiation, or both.

Two exemplary instances of this construct type are presented in FIGS. 9E-F. In FIG. 9E (left panel), an RNA construct containing two AptTBP-12 aptamers having a nucleotide sequence corresponding to SEQ ID NO: 55 as follows:

GGGAGAAUUC AACUGCCAUC UAGGCGCCGU GCCCGGUUUG GAUAGGCACA UAAGACGCCG
ACAAAGAAAC CAACCAGUAC UACAAGCUUC UGGACUCGGU

(formerly named #12 in Fan et al., “Probing TBP Interactions in Transcription Initiation and Reinitiation with RNA Aptamers that Act in Distinct Modes,” PNAS 101(18):6934-6939 (2004), which is hereby incorporated by reference in its entirety) and one MS2 binding site is illustrated. Referred to as AptTBP12(2)MS2(1), this construct has a nucleotide sequence corresponding to SEQ ID NO: 45 as follows:

UGGGCUAAGC CCACUGAUGA GUCGCUGAAA UGCGACGAAA CCUCGAGUCA UACUCGCGGC
CGCUGACGCG CCGUGCCCGG UUUGGAUAGG CACAUAAGAC GCGUCAUACU CCGCCGUGCC
CGGUUUGGAU AGGCACAUAA GACGGAGGGC GGCCGCGAGG CGGCAGUAUU AAACAUGAGG
AUCACCCAUG UCCAGUACUG CCGUGACUCG ACGUC

In FIG. 9E (right panel), a precursor of the construct illustrated in FIG. 9E (left panel) prior to cleavage by two cis-acting hammerhead ribozymes is shown. This is a transcript from the yeast RPR 1 promoter. The two ribozymes are derived respectively from the peach latent mosaic viroid and the tobacco ringspot virus satellite RNA. This precursor constructed has a nucleotide sequence corresponding to SEQ ID NO: 52 as follows:

GUUUUACGUU UGAGGCCUCG UGGCGCACAU GGUACGCUGU GGUGCUCGCG GCUGGGAACG
AAACUCUGGG AGCUGCGAUU GGCAGAAUUC ACUUGAGGUC UGGGCUAAGC CCACUGAUGA
GUCGCUGAAA UGCGACGAAA CCUCGAGUCA UACUCGCGGC CGCUGACGCG CCGUGCCCGG
UUUGGAUAGG CACAUAAGAC GCGUCAUACU CCGCCGUGCC CGGUUUGGAU AGGCACAUAA
GACGGAGGGC GGCCGCGAGG CGGCAGUAUU AAACAUGAGG AUCACCCAUG UCCAGUACUG
CCGUGACUCG ACGUCUAGCG AUGUGGUUUC GCUACUGAUG AGUCCGUGAG GACGAAACGU
CGACGAAUUC CCCCAUAUCC AACUUCCAAU UUAAUCUUUC UUUUU

In FIG. 9F (left panel), an RNA construct containing one AptTBP-101 aptamer (described in Example 13) and two MS2 binding sites is illustrated. Referred to as AptTBP101(1)MS2(2), this construct has a nucleotide sequence corresponding to SEQ ID NO: 46 as follows:

UGGGCUAAGC CCACUGAUGA GUCGCUGAAA UGCGACGAAA CCUCGAGUCA UACUCGCGGC
CGCAGAAUUC AACUCUUCGG AGCCAAGGUA AACAAUUCAG UUAGUGGAAU GAAACUGGCG
GCCGCGAGGC GGCAGUAUUC CGGUUCGCGC AGAAACAUGA GGAUCACCCA UGUCCUGUGC
CACAGCGGUG AAACAUGAGG AUCACCCAUG UCCACCAGCG UUCCGGAGUA CUGCCGUGAC
UCGACGUC

In FIG. 9F (right panel), a precursor of the construct illustrated in FIG. 9F (left panel) prior to cleavage by two cis-acting hammerhead ribozymes is shown. This is a transcript from the yeast RPR 1 promoter. The two ribozymes are derived respectively from the peach latent mosaic viroid and the tobacco ringspot virus satellite RNA. This precursor constructed has a nucleotide sequence corresponding to SEQ ID NO: 53 as follows:

GUUUUACGUU UGAGGCCUCG UGGCGCACAU GGUACGCUGU GGUGCUCGCG GCUGGGAACG
AAACUCUGGG AGCUGCGAUU GGCAGAAUUC ACUUGAGGUC UGGGCUAAGC CCACUGAUGA
GUCGCUGAAA UGCGACGAAA CCUCGAGUCA UACUCGCGGC CGCAGAAUUC AACUCUUCGG
AGCCAAGGUA AACAAUUCAG UUAGUGGAAU GAAACUGGCG GCCGCGAGGC GGCAGUAUUC
CGGUUCGCGC AGAAACAUGA GGAUCACCCA UGUCCUGUGC CACAGCGGUG AAACAUGAGG
AUCACCCAUG UCCACCAGCG UUCCGGAGUA CUGCCGUGAC UCGACGUCUA GCGAUGUGGU
UUCGCUACUG AUGAGUCCGU GAGGACGAAA CGUCGACGAA UUCCCCCAUA UCCAACUUCC
AAUUUAAUCU UUCUUUUU

Materials and Methods for Example 13

Protein Preparation

The tagged Drosophila HSF constructs were expressed and purified from E. coli as described previously (Mason et al., “Cooperative and competitive protein interactions at the hsp70 promoter,” J. Biol. Chem. 272(52):33227-33 (1997), which is hereby incorporated by reference in its entirety). Recombinant yeast TBP and yeast TFIIA were prepared respectively as described (Fan et al., “Probing TBP Interactions in Transcription Initiation and Reinitiation with RNA Aptamers that Act in Distinct Modes,” PNAS 101(18):6934-6939 (2004), which is hereby incorporated by reference in its entirety). Recombinant yeast TFIIB was provided by Drs. J. Fu and M. H. Suh (Cornell University, Ithaca, N.Y.).

In Vitro Evolution

The two starting pools had the same sequences in the constant regions but differed in the length of randomized region. The template-primer system was described previously (Shi et al., “A Specific RNA Hairpin Loop Structure Binds the RNA Recognition Motifs of the Drosophila SR Protein B52,” Mol. Cell. Biol. 17(5):2649-57 (1997), which is hereby incorporated by reference in its entirety). Selection with HSF and TBP used pools with 40 or 50 randomized positions respectively. The procedure of selection and amplification were almost identical to that described previously (Shi et al., “A Specific RNA Hairpin Loop Structure Binds the RNA Recognition Motifs of the Drosophila SR Protein B52,” Mol. Cell. Biol. 17(5):2649-57 (1997); Fan et al., “Probing TBP Interactions in Transcription Initiation and Reinitiation with RNA Aptamers that Act in Distinct Modes,” PNAS 101(18):6934-6939 (2004), which are hereby incorporated by reference in their entirety). When a negative selection against the filters was included in a selection cycle, the retrieved “bound” RNA from the filter was reconstituted in binding buffer with potassium and passed, through a filter. The “unbound” flow-through was collected and amplified by RT-PCR. RNA restriction (negative selection according to genotype) was performed as described previously (Shi et al., “Evolutionary Dynamics and Population Control During In Vitro Selection and Amplification with Multiple Targets,” RNA 8(11):1461-70 (2002), which is hereby incorporated by reference in its entirety). To eliminate the MGMs in the experiment involving HSF, the first two treatments were done with the “dodecamarker” set and the last included an additional marking oligo, NCW13 (Shi et al., “Evolutionary Dynamics and Population Control During In Vitro Selection and Amplification with Multiple Targets,” RNA 8(11):1461-70 (2002), which is hereby incorporated by reference in its entirety). To mask the DNA binding surface of TBP, a “mini” version of AptTBP-12 (m 12) or the TATA-DNA was included in the binding reaction in excess to TBP. They were incubated with TBP for 30 minutes before adding the RNA pool to the mix. The m12 was chemically synthesized by Dharmacon and has the following sequence:

(SEQ ID NO: 51)

5′-GGCGCCGUGCCCGGUUUGGAUAGGCACAUAAGACGCC-3′.

Southern Dot Blot Analysis

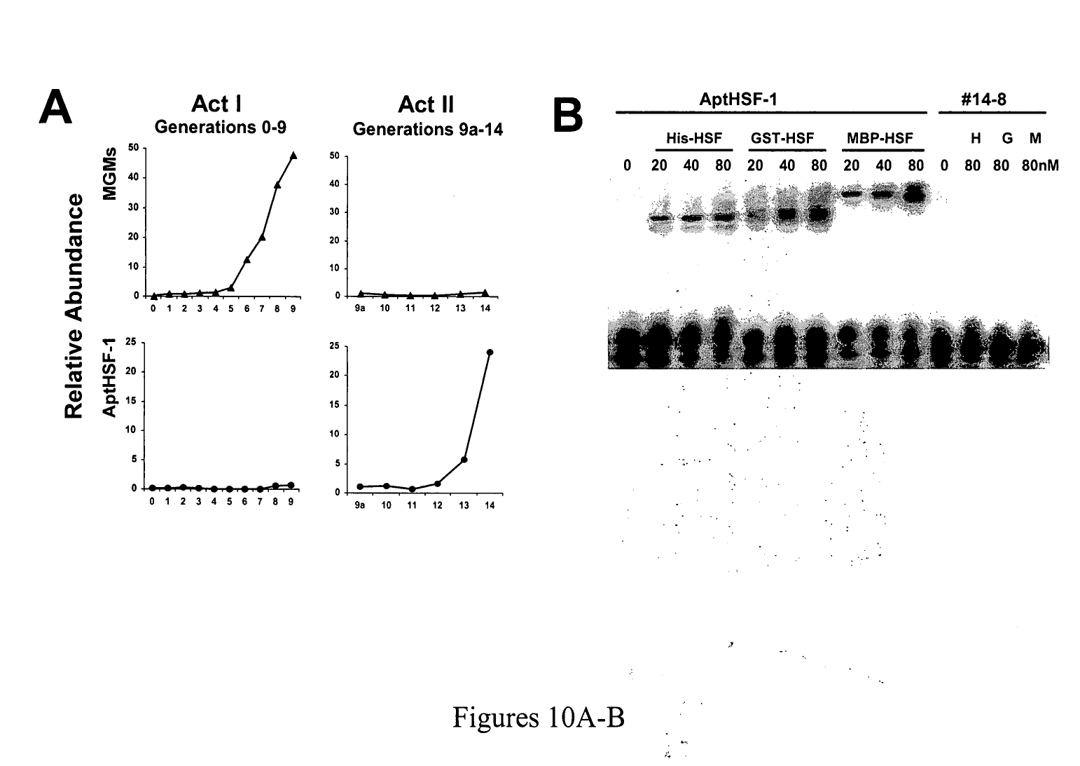

The probe for MGM was the same as used in a previous study (Shi et al., “Evolutionary Dynamics and Population Control During In Vitro Selection and Amplification with Multiple Targets,” RNA 8(11): 1461-70 (2002), which is hereby incorporated by reference in its entirety). The probes for HSF aptamers were oligonucleotide 40-bases in length derived from the randomized region of RA1-HSF (shown in capital letters in FIG. 10C). The procedure was identical to that described previously (Shi et al., “Evolutionary Dynamics and Population Control During In Vitro Selection and Amplification with Multiple Targets,” RNA 8(11):1461-70 (2002), which is hereby incorporated by reference in its entirety).

Electrophoretic Mobility Shift (EMS) Assay

All RNA-protein binding assays were performed in 20 μl reaction volumes. Binding with HSF and TBP were performed respectively as previously described (Shi et al., “A Specific RNA Hairpin Loop Structure Binds the RNA Recognition Motifs of the Drosophila SR Protein B52,” Mol. Cell. Biol. 17(5):2649-57 (1997); Fan et al., “Probing TBP Interactions in Transcription Initiation and Reinitiation with RNA Aptamers that Act in Distinct Modes,” PNAS 101(18):6934-6939 (2004), which are hereby incorporated by reference in their entirety). When EMS assay is used as a partitioning method, the ratio of RNA to protein was 10:1, and the shifted band were located by a radiolabeled RNA probe in a separate reaction run on the same gel. Binding reactions with TBP contained 0.5 nM labeled aptamer and 25 nM yeast TBP. Where indicated, TATA-DNA was included at 50 nM, other recombinant proteins at 200 nM, DNaseI at 0.25 U/μl, and ProteaseK at 1 μg/μl. The running buffer for TBP.aptamer complexes was 0.5×TG with 0.5 mM magnesium acetate.

In Vitro Transcription

Transcription was performed according to a previously described protocol (Fan et al., “Probing TBP Interactions in Transcription Initiation and Reinitiation with RNA Aptamers that Act in Distinct Modes,” PNAS 101(18):6934-6939 (2004), which is hereby incorporated by reference in its entirety).

### Example 13

### Identification and Isolation of an Aptamer Directed to Multiple Sites on the TATA-Binding Protein

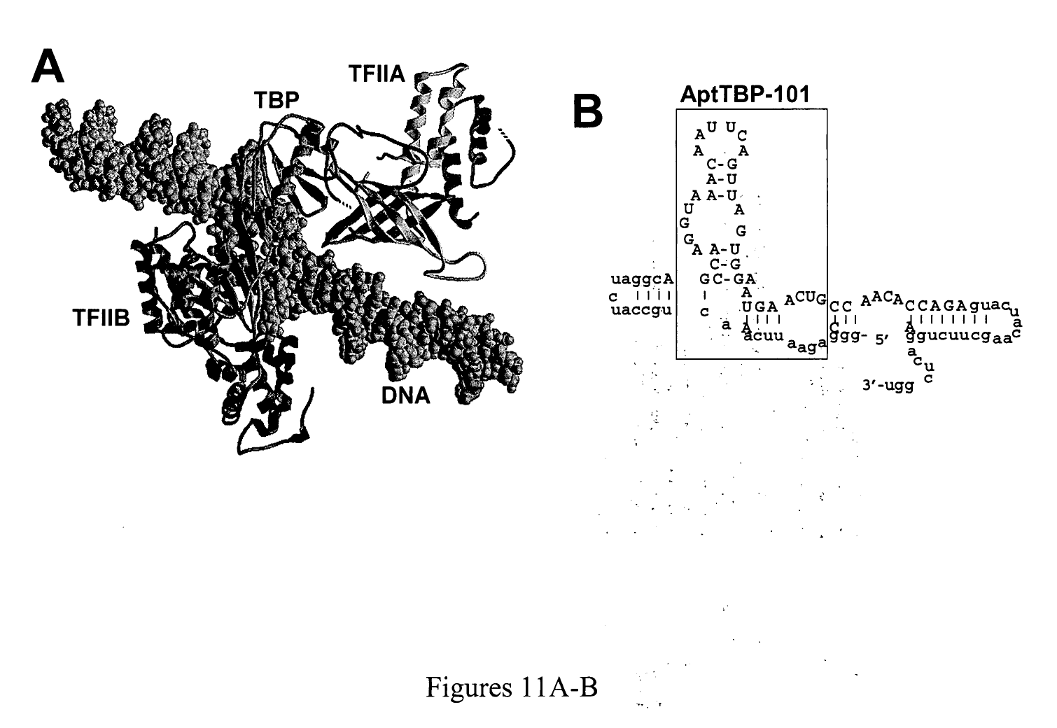

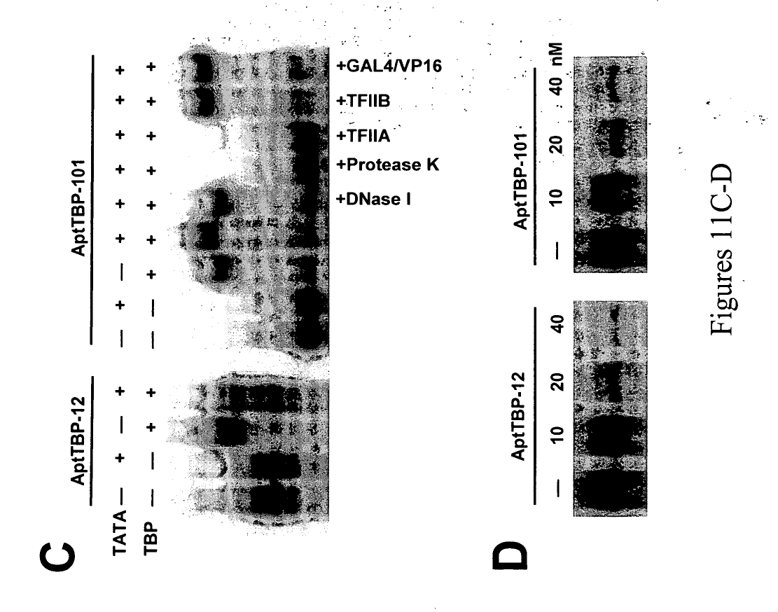

TATA-binding protein (“TBP”) is a universal transcription factor that is involved in transcription by all three types of eukaryotic RNA polymerases. By interacting directly with other factors that can activate or repress transcription, it also functions as a hub of gene regulation (reviewed in Pugh, “Control of Gene Expression through Regulation of the TATA-Binding Protein,” Gene 255(1):1-14 (2000), which is hereby incorporated by reference in its entirety). The conserved C-terminal core domain of TBP is less than 30 kD in size, and has a pseudo-symmetric saddle shape. Multiple functional sites have been mapped on TBP as small clusters of amino acids (Tang et al., “Protein-Protein Interactions in Eukaryotic Transcription Initiation: Structure of the Preinitiation Complex,” Proc. Natl. Acad. Sci. USA 93(3):1119-24 (1996), which is hereby incorporated by reference in its entirety). As shown in FIG. 11A, the concave surface of TBP primarily interacts with the TATA-element and induces a sharp bend in the DNA (Chasman et al., “Crystal Structure of Yeast TATA-Binding Protein and Model for Interaction with DNA,” Proc. Natl. Acad. Sci. USA 90(17):8174-8 (1993), which is hereby incorporated by reference in its entirety), and its convex side is recognized by many transcription activators and suppressors (Burley et al., “Biochemistry and Structural Biology of Transcription Factor IID (TFIID),” Annu. Rev. Biochem. 65:769-99 (1996), which is hereby incorporated by reference in its entirety). Based on these features, it is anticipated that a conventional SELEX would yield aptamers for the concave, DNA-binding, side of the molecule.

A new RNA pool that was estimated to contain more than 1015 individuals was carried through 12 cycles of selection and amplification with yeast TBP. A sample of 32 individuals yielded 8 clones of aptamers belonging to a single class: they all recognize the DNA-binding side of the molecule. As documented elsewhere (Fan et al., “Probing TBP Interactions in Transcription Initiation and Reinitiation with RNA Aptamers that Act in Distinct Modes,” Proceedings of the National Academy of Sciences 101(18):6934-6939 (2004), which is hereby incorporated by reference in its entirety), these aptamers acted in distinct modes and served as useful probes to yield rich information about TBP interaction during transcription initiation and reinitiation. However, while these results further motivated the search for aptamers directed to other sites on TBP, the presence of many families in this class (designated Class 1) made it less practical to divert aptamers to other sites in subsequent evolution.

A pool of early generation selected by yeast TBP under conventional conditions was taken, the third generation (G3) in the previous experiment (Fan et al., “Probing TBP Interactions in Transcription Initiation and Reinitiation with RNA Aptamers that Act in Distinct Modes,” Proceedings of the National Academy of Sciences 101(18):6934-6939 (2004), which is hereby incorporated by reference in its entirety), and selection and amplification was performed with the natural ligand of TBP, the TATA-DNA, included in the binding mixture. After four generations, it was found that a single clone, designated AptTBP-101, dominated the selected pool (6 members out of 6 individuals sequenced), and this clone was not isolated in the selection without TATA-DNA (Fan et al., “Probing TBP Interactions in Transcription Initiation and Reinitiation with RNA Aptamers that Act in Distinct Modes,” Proceedings of the National Academy of Sciences 101(18):6934-6939 (2004), which is hereby incorporated by reference in its entirety). The Apt TBP-101 aptamer has a nucleotide sequence corresponding to SEQ ID NO: 47 as follows:

GGGAGAAUUC AACUGCCAUC UAGGCAGCCA AGGUAAACAA UUCAGUUAGU GGAAUGAAAC
UGCCCAACAC CAGAAGUACU ACAAGCUUCU GGACUCGGU

To further demonstrate the utility of this method in cases where no natural ligand is available, an evolution from G3 was performed in parallel with a previously isolated Class 1 aptamer included in place of the TATA-DNA. AptTBP-12 was reduced to a form that fully retained its binding capability without the constant regions, so it would not be amplified along with the candidates during this stage of evolution. Four generations later, the same clone, AptTBP-101, was isolated.

The affinity of AptTBP-101 to TBP was measured with a filter-binding assay. The Kd of AptTBP-101.TBP complex was 2 nM, similar to that of the AptTBP-12.TBP complex. Based on the predicted secondary structure, a series of variants was made to confirm this structure and map the “true” aptamer moiety. Although they have different binding specificity and their randomized regions are different in composition and in length, the secondary structure of AptTBP-101 and RA1-HSF share similar features. As shown in FIGS. 10C and 11B, they both have a three-way junction, an asymmetric internal loop and an apical loop; and they both made use of a segment in the 5′ constant region in a similar manner.

The first indication that AptTBP-101 indeed bound to somewhere other than the DNA binding site, and thus belongs to a different class, came from an EMS assay. When TBP was present, all Class 1 aptamers generated a shifted band with identical mobility. But AptTBP-101 caused a shift with different mobility, as shown in FIG. 11C. Moreover, in contrast to AptTBP-12, which competed with TATA-DNA in binding to TBP, AptTBP-101 was able to generate a “super-shift” in the presence of the TATA-DNA, indicating the formation of a triple-complex with both TBP and TATA-DNA. The identity of this RNA-DNA-Protein complex was verified by its sensitivity to DNase I and Protease K, which clearly demonstrated that AptTBP-101 binding site did not overlap with the DNA binding site. To further pinpoint the binding site of AptTBP-101, its ability to compete with other proteins that recognize TBP in the TBP-TATA complex was tested. TFIIA, but not TFIIB or Gal4/VP16, was able to block the binding of AptTBP-101 when present in excess. Therefore, the binding site of AptTBP-101 must overlap with the site recognized by TFIIA. Referring to the model in FIG. 11A, this site is on the side of TBP opposite to the side that binds TFIIB.

Several Class 1 aptamers were able to inhibit RNA polymerase II (“Pol II”) dependent transcription efficiently (Fan et al., “Probing TBP Interactions in Transcription Initiation and Reinitiation with RNA Aptamers that Act in Distinct Modes,” PNAS 101(18):6934-6939 (2004), which is hereby incorporated by reference in its entirety). The effects of the Class 2 aptamer AptTBP-101 was also tested in a similar in vitro assay. When a DNA template containing an Adenovirus major late (“AdML”) promoter was used with yeast whole cell extracts, as shown in FIG. 11D, AptTBP-101 inhibited Pol II dependent transcription as potently as AptTBP-12, when they were present during the process of preinitiation complex (PIC) formation. Since AptTBP-101 has little effect on TBP.TATA interaction and mainly interfered with interaction between TBP and TFIIA, this result suggested that the TBP.TFIIA interaction was important for basal transcription of some genes, at least in the in vitro transcription system. Alternatively, it is also possible that the binding of AptTBP-101 to TBP precluded the recruitment of other factors such as Pol II or TFIIF.

### Prophetic Example 14

### Generation of Multiple Aptamers Directed to Different Transcription Factors and Different Sites on Single Transcription Factors

In a formal sense, almost all in vitro evolution experiments involve multiple targets. The target set can be a mixture of multiple distinctive molecules, a single molecule containing multiple discrete binding sites on its surface, or a presumably pure target preparation with unknown contaminants. A conventional single-target selection scheme often requires a partitioning matrix (e.g., filters used to collect RNA-protein complexes) that functions as an unwanted target largely inseparable from the intended one. To isolate aptamers for multiple targets, a model of in vitro evolution with changing fitness landscape is proposed and two types of experimental manipulations that execute these changes have been devised.

An RNA aptamer for a protein or other types of targets often mimics the shape of a natural ligand. Although RNA is an extraordinarily versatile type of molecule, it can not be guaranteed that an RNA ligand always exists for a particular binding site on a protein domain naturally recognized by a non-RNA molecule. On the other hand, multiple different RNA sequence/structure solutions may exist to fit a single site, as seen in the TBP aptamers that bind the DNA-binding surface (Fan et al., “Probing TBP Interactions in Transcription Initiation and Reinitiation with RNA Aptamers that Act in Distinct Modes,” PNAS 101(18):6934-6939 (2004), which is hereby incorporated by reference in its entirety). The approach pursues exhaustiveness in aptamer identification in a practical sense: every RNA ligand existing in the starting sequence pool can be selected using the procedures described above and disclosed in U.S. Patent Application Publication No. 2004/0053310 to Shi et al., which is hereby incorporated by reference in its entirety. In the scheme, the most fit aptamer clone or clones are converted to the least fit one(s) after their identification, thus allowing clones to dominate the selected pools in successive stages in an order according to their original rank of fitness. This scheme should be equally viable for DNA aptamers.

The change of the relative growth rate of a clone is executed using identified aptamers. During further evolution, their genotype (sequence information) is used to decrease the viability of individuals in the same clone, and their phenotype (affinity determined by their shapes) is utilized to decrease the availability of their targets. These two methods can be combined in a single experiment to achieve desirable results. The ease of use allows a systematic search for aptamers binding to transcription factors. While AptTBP-101 was isolated by “masking” the DNA-binding surface with AptTBP-12, an aptamer binding to yet another site on TBP was isolated from a pool dominated by AptTBP-101, by eliminating this clone using RNaseH treatment with a DNA oligonucleotide bearing the unique sequence of AptTBP-101.

The two aptamers described above, RA1-HSF and AptTBP-101, stemmed from different ancestral pools, were selected for disparate targets, and possessed unrelated activities. Yet their secondary structures share striking similarities. In particular, in both cases an almost identical gap in the sequence of the “true” aptamer was identified—the loop consisting of 5′ constant sequences. The constant regions were included for the purpose of amplification, not preordained to be part of an aptamer for a specific target. However, utilizing small segments in the constant regions increased the information content and structural complexity of an aptamer, which possibly resulted in a higher affinity (Carothers et al., “Informational Complexity and Functional Activity of RNA Structures,” J. Am. Chem. Soc. 126(16):5130-7 (2004), which is hereby incorporated by reference in its entirety).

The newly developed methods reported here allowed the generation of multiple aptamers directed to different transcription factors and different sites on single transcription factors. The availability of these aptamers will enable the manipulation of the process of transcription in vivo with spatial and temporal precision. General methods to regulate the production of aptamers in vivo has been established (Shi et al., “RNA Aptamers as Effective Protein Antagonists in a Multicellular Organism,” Proc. Natl. Acad. Sci. USA 96(18):10033-8 (1999); U.S. Patent Application Publication No. 2004/0053310 to Shi et al., which are hereby incorporated by reference in their entirety), which can be coupled with the control of aptamer activity through allosteric transitions (Buskirk et al., “Engineering a Ligand-Dependent RNA Transcriptional Activator,” Chem. Biol. 11 (8): 1157-63 (2004), which is hereby incorporated by reference in its entirety). Using these reagents and methods, “plug-and-play” molecular devices are constructed to test the effects of perturbing functions of many proteins, especially those “hubs” in regulatory networks, in different ways and in different combinations.

All aptamers mentioned herein can be incorporated readily into aptabodies, as described supra in Examples 7, 8, and 9, by applying the design procedure set forth therein. While the aptabodies described in these examples are simple mimics of monoclonal antibodies, the design principle of aptabodies can also yield constructs with functionality beyond the capability of antibodies. As examples, two instances with combinatorial specificities directed to close target proximity are presented.

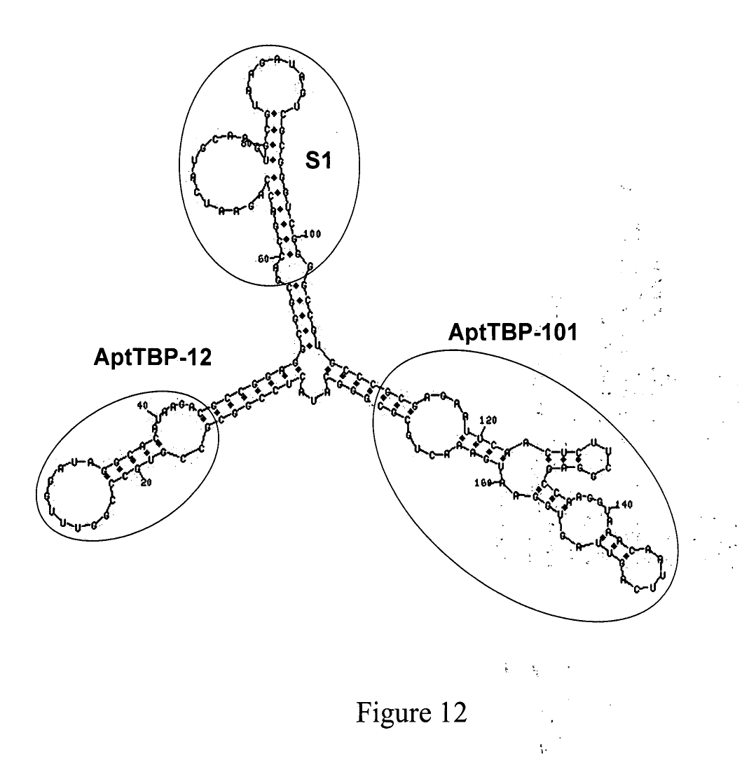

The first type of the aptabody variants contains multiple aptamers directed to distinct sites on a single protein target, thus functioning as a chelator. An example of this type comprises one Streptavidin aptamer, S1, and two TBP aptamers, AptTBP12 and AptTBP101, as depicted in FIG. 12. The two TBP aptamers, as shown in Example 13, bind to different sites of their target. This construct has the following sequences:

Aptabody-12/S1/101 has a nucleotide sequence corresponding to SEQ ID NO: 48 as follows:

GGGCAUACUC CGGCGCCGUG CCCGGUUUGG AUAGGCACAU AAGACGCCGG AGGCGGCGAC
CGACCAGAAU CAUGCAAGUG CGUAAGAUAG UCGCGGGUCG GGGCCGUGCC CGCGAGAAUU
CAACUCUUCG GAGCCAAGGU AAACAAUUCA GUUAGUGGAA UGAAACUGCG C

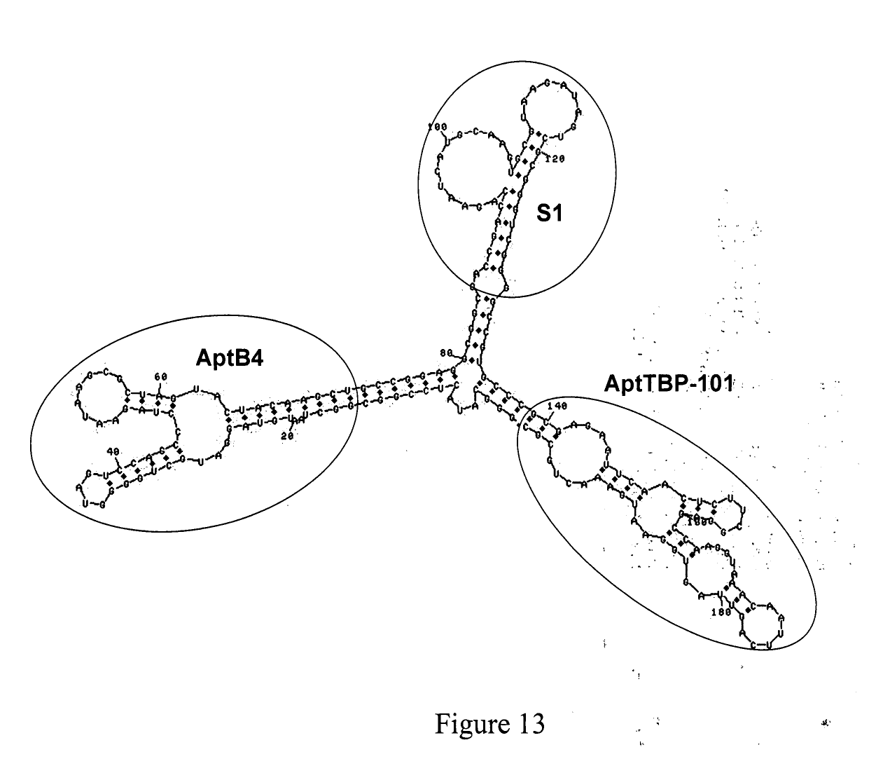

The second type of the aptabody variants contains aptamers directed to multiple protein targets in a supramolecular assembly and functions as complex-specific antibodies. As depicted in FIG. 13, an example of this type comprises one Streptavidin aptamer, S1, one TBP aptamer, AptTBP101, and one aptamer directed to TFIIB, AptB4, which has a nucleotide sequence corresponding to SEQ ID NO: 50 as follows:

GGGAGAAUUC AACUGCCAUC UAGGCAAAGA GCUAAUGUAG GAUGCUGGGG UAGUCCAGCC
CUAGAAUAAG CGCUAGUACU ACAAGCUUCU GGAGCUCGGU

TFIIB is a general transcription factor of RNA polymerase II. The aptamer AptB4 has an affinity to TFIIB at 7 nM. The binding of AptB4 does not interfere with the interaction between TFIIB and TBP. Hence, this construct is specific not only to TBP or TFIIB, but also to the TATA DNA.TBP.TFIIB complex, which is a partial pre-initiation complex formed in early steps of transcription initiation. And, the avidity of this construct to the TATA DNA.TBP.TFIIB complex is expected to exceed that to either TBP or TFIIB along. This construct has the following sequences:

Aptabody-B4/S1/101, which has a nucleotide sequence corresponding to SEQ ID NO: 49 as follows:

GGGCAUACUC CGGCGGCUAA UGUAGGAUGC UGGGGUAGUC CAGCCCUAGA AUAAGCGCUA
GUACUACAAG CUGCCGGAGG CGGCGACCGA CCAGAAUCAU GCAAGUGCGU AAGAUAGUCG
CGGGUCGGGG CCGUGCCCGC GAGAAUUCAA CUCUUCGGAG CCAAGGUAAA CAAUUCAGUU
AGUGGAAUGA AACUGCGC

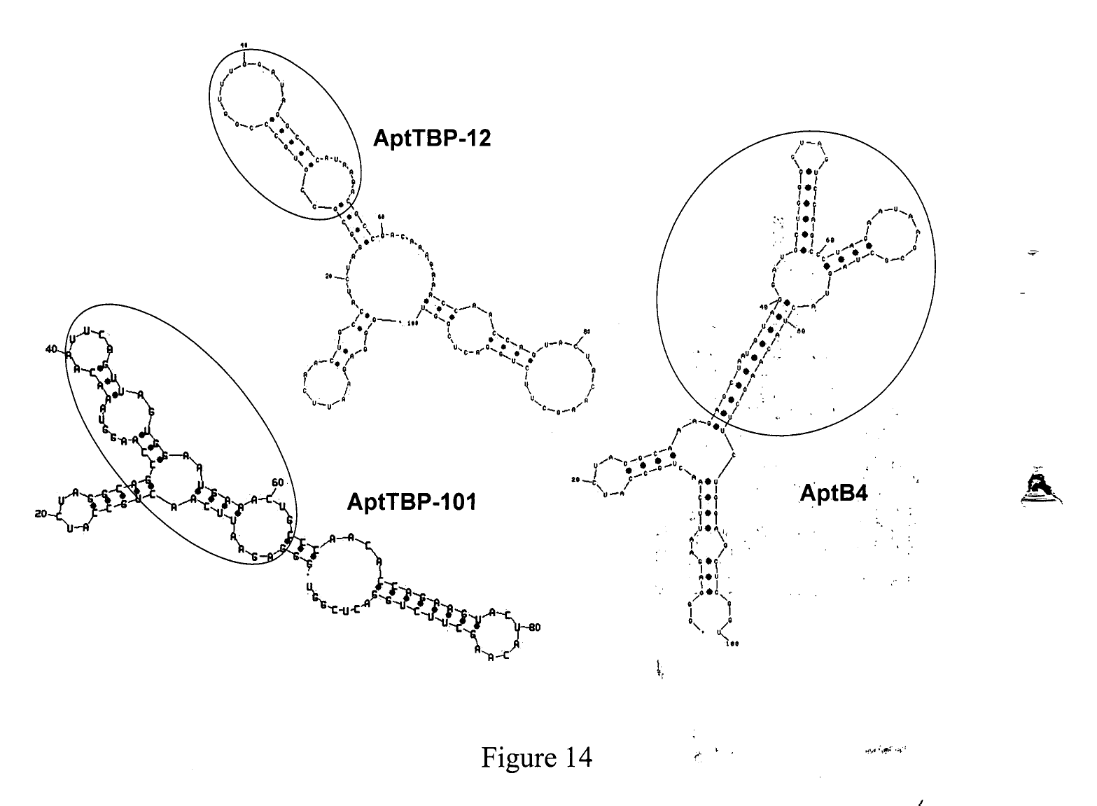

FIG. 14 depicts the secondary structure of the building-block aptamers in the form in which they were isolated from a combinatorial pool. The structures in the circles were confirmed by mutational studies to be the active aptamer moieties. Comparing these structures with corresponding parts annotated in FIGS. 12 and 13 demonstrates the successful preservation of these structures, and in turn functions thereof, in the new context of aptabodies.

Although the invention has been described in detail for the purpose of illustration, it is understood that such detail is solely for that purpose, and variations can be made therein by those skilled in the art without departing from the spirit and scope of the invention which is defined by the following claims.

1. A nucleic acid molecule comprising:
first and second nucleic acid elements that each bind a target molecule and
a three-way junction comprised of the same type of nucleic acid as the first and second nucleic acid elements, wherein the three-way junction operably links the first and second nucleic acid elements.

2. The nucleic acid molecule according to claim 1 further comprising:
a plurality of three-way junctions (n), wherein n is a positive integer greater than 1 and
a plurality of nucleic acid elements (≦n+2), each of which binds a target molecule,
wherein each of the nucleic acid elements is operably linked to a three-way junction, and wherein each of the three-way junctions is comprised of the same type of nucleic acid as the nucleic acid elements and is operably linked to another of the plurality of three-way junctions by a linker region.

3. The nucleic acid molecule according to claim 1, wherein the first and second nucleic acid elements are RNA aptamers or DNA aptamers.

4. The nucleic acid molecule according to claim 1, wherein the three-way junction comprises three double-stranded stems radiating from a junction region, wherein each stem comprises two or more consecutive, canonical base-pairs between anti-parallel strands.

5. The nucleic acid molecule according to claim 1, wherein the first and second nucleic acid elements each bind distinct target molecules or distinct sites on a single target molecule.

6. The nucleic acid molecule according to claim 1, wherein the first and second nucleic acid elements each bind the same target molecule.

7. The nucleic acid molecule according to claim 1, wherein the three-way junction is selected from the group consisting of Loop A and any associated 5S RNA, System D, and System F.

8. The nucleic acid molecule according to claim 1, wherein the termini of the nucleic acid molecule are resistant to exonucleases.

9. The nucleic acid molecule according to claim 1 further comprising:
third and fourth nucleic acid elements that each bind a target molecule; and
a second three-way junction operably linking the third and fourth nucleic acid elements to the first three-way junction.

10. The nucleic acid molecule according to claim 9, wherein the third and fourth nucleic acid elements are RNA aptamers or DNA aptamers.

11. The nucleic acid molecule according to claim 9 further comprising:
a nucleic acid linker region between the first and second three-way junctions.

12. The nucleic acid molecule according to claim 9, wherein the first and second nucleic acid elements bind a first target molecule and the third and fourth nucleic acid elements bind a second target molecule that is distinct from the first target molecule.

13. The nucleic acid molecule according to claim 1, wherein the nucleic acid molecule is RNA.

14. The nucleic acid molecule according to claim 1, wherein the nucleic acid molecule is DNA.

15. A constructed DNA molecule encoding an RNA molecule according to claim 13.

16. An expression system comprising an expression vector into which is inserted a DNA molecule according to claim 15.

17. A host cell containing a DNA molecule according to claim 15.

18. A constructed DNA molecule comprising the DNA molecule according to claim 14.

19. A host cell containing the DNA molecule according to claim 14.

20. An engineered gene comprising:
a DNA sequence encoding an RNA molecule according to claim 13; and
a regulatory sequence operably coupled to the DNA sequence to control expression of the encoded RNA molecule.

21. The engineered gene according to claim 20, wherein the DNA sequence comprises a plurality of monomeric DNA sequences linked together in a single DNA chain, each of the monomeric DNA sequences encoding one of the RNA molecules.

22. The engineered gene according to claim 21, wherein each of the plurality of monomeric DNA sequences also encodes a cis-acting ribozyme.

23. An expression system comprising an expression vector into which is inserted an engineered gene according to claim 20.

24. A host cell containing an engineered gene according to claim 20.

25. A transgenic non-human organism whose somatic and/or germ cell lines contain an engineered gene according to claim 20.

26. An RNA scaffold comprising:
first and second RNA receptor regions operably linked by a three-way junction, wherein the first and second RNA receptor regions each comprise a stem defined by at least two sets of consecutive, canonic paired bases.

27. The RNA scaffold according to claim 26 further comprising:
a plurality of three-way junctions (n), wherein n is a positive integer greater than 1 and
a plurality of receptor regions (≦n+2), wherein each of the receptor regions is operably linked to a three-way junction, each receptor region being comprised of a stem defined by at least two sets of consecutive, canonic paired bases, and wherein each of the three-way junctions is operably linked to at least one of the plurality of three-way junctions by a linker region.

28. The RNA scaffold according to claim 26 further comprising:
a third RNA receptor region operably linked to the three-way junction, said third RNA receptor region comprising a stem defined by at least two sets of consecutive, canonic paired bases.

29. The RNA scaffold according to claim 26 further comprising:
an RNA structure resistant to exonuclease digestion operably linked to the three-way junction.

30. The RNA scaffold according to claim 26 further comprising:
third and fourth RNA receptor regions operably linked by a second three-way junction, the second three-way junction being joined to the first three-way junction by a linker region, wherein the third and fourth RNA receptor regions each comprise a stem defined by at least two sets of consecutive, canonic paired bases.

31. A functional RNA molecule comprising:
an RNA scaffold according to claim 26; and
one or more functional modules operably linked to one or more of the RNA receptor regions.

32. The functional RNA molecule according to claim 31, wherein the functional modules serve a function selected from the group consisting of: accumulation, stability, aptamer presentation, oligomerization, transportation, and localization.

33. The functional RNA molecule according to claim 31, wherein each of the functional modules is independently selected from the group of ribozyme, RNA aptamer, DNA aptamer, a tetraloop receptor, a transport element, and a target ligand.

34. A constructed DNA molecule encoding an RNA scaffold according to claim 26.

35. An expression system comprising an expression vector into which is inserted a DNA molecule according to claim 34.

36. A host cell containing a DNA molecule according to claim 34.

37. An engineered gene encoding an RNA scaffold comprising:
a DNA sequence encoding an RNA scaffold according to claim 26; and
a regulatory sequence which controls expression of the DNA sequence encoding an RNA scaffold.

38. An expression system comprising an expression vector into which is inserted an engineered gene according to claim 37.

39. A host cell containing an engineered gene according to claim 37.

40. A transgenic non-human organism whose somatic and/or germ cell lines contain an engineered gene encoding the RNA scaffold according to claim 26.


SEQUENCE LISTING (50 sequences)

SEQ ID NO: 1 - DNA, 76 bp, Artificial Sequence
  Description of Artificial Sequence transcription template BBS-5′
gtaatacgactcactatagggatcgccgcggctggtcaaccaggcgaccgccgcggccacagcggtgggctggtca

SEQ ID NO: 2 - DNA, 78 bp, Artificial Sequence
  Description of Artificial Sequence transcription template BBS-3′
gaatcccgaaggatccgggaacgctggtgggcggtcgcctggttgaccagcccaccgctgtggccgcggcggtcgcct

SEQ ID NO: 3 - DNA, 80 bp, Artificial Sequence
  Description of Artificial Sequence transcription template SAa-5′
gtaatacgactcactataggatccgtgaccgaccagaatcatgcaagtgcgtaagatagtcgcgggtcgggtcatactcc

SEQ ID NO: 4 - DNA, 80 bp, Artificial Sequence
  Description of Artificial Sequence transcription template SAa-3′
gaatccgcctcccggcccgcgactatcttacgcacttgcatgattctggccgggagtatgacccgacccgcgactatctt

SEQ ID NO: 5 - DNA, 65 bp, Artificial Sequence
  Description of Artificial Sequence transcription template TAR Mal-5′
gtaatacgactcactatagggatcgccgccgagcccgggagctcggcggccacagcggtgggagc

SEQ ID NO: 6 - DNA, 60 bp, Artificial Sequence
  Description of Artificial Sequence transcription template TAR Mal-3′
gaatcccgaaggatccgggaacgctggtgggagctcccgggctcccaccgctgtggccgc

SEQ ID NO: 7 - DNA, 62 bp, Artificial Sequence
  Description of Artificial Sequence transcription templace R-06-5′
gtaatacgactcactataggatccgtgacgtcaacacggtcccggacgtgttgacgtccata

SEQ ID NO: 8 - DNA, 60 bp, Artificial Sequence
  Description of Artificial Sequence transcription template R-06-3′
gaatccgcctcctcaacacgtccgggaccgtgttgaggagtatggacgtcaacacgtccg

SEQ ID NO: 9 - DNA, 45 bp, Artificial Sequence
  Description of Artificial Sequence transcription template 5′S1cons
gtaatacgactcactatagggaccgaccagaatcatgcaagtgcg

SEQ ID NO: 10 - DNA, 45 bp, Artificial Sequence
  Description of Artificial Sequence transcription template 3′S1cons
cccggcccgcgactatcttacgcacttgcatgattctggtcggtc

SEQ ID NO: 11 - DNA, 217 bp, Artificial Sequence
  Description of Artificial Sequence transcription templace Temp-BS
gtaatacgactcactatagggatcgccgcggctggtcaaccaggcgaccgccgcggccacagcggtgggctggtcaaccaggcgaccgcccaccagcgttcccggatccgtgaccgaccagaatcatgcaagtgcgtaagatagtcgcgggtcgggtcatactcccggccagaatcatgcaagtgcgtaagatagtcgcgggccgggaggcggattc

SEQ ID NO: 12 - DNA, 187 bp, Artificial Sequence
  Description of Artificial Sequence transcription template Temp-BR
gtaatacgactcactatagggatcgccgcggctggtcaaccaggcgaccgccgcggccacagcggtgggctggtcaaccaggcgaccgcccaccagcgttcccggatccgtgacgtcaacacggtcccggacgtgttgacgtccatactcctcaacacggtcccggacgtgttgaggaggcggattc

SEQ ID NO: 13 - DNA, 203 bp, Artificial Sequence
  Description of Artificial Sequence transcription template Temp-TS
gtaatacgactcactatagggatcgccgccgagcccgggagctcggcggccacagcggtgggagcccgggagctcccaccagcgttcccggatccgtgaccgaccagaatcatgcaagtgcgtaagatagtcgcgggtcgggtcatactcccggccagaatcatgcaagtgcgtaagatagtcgcgggccgggaggcggattc

SEQ ID NO: 14 - RNA, 84 nt, Artificial Sequence
  Description of Artificial Sequence RNA Aptamer S1
gggagucgaccgaccagaaucaugcaagugcguaagauagucgcgggccgggggcguauuaugugcgucuacaucuagacucau

SEQ ID NO: 15 - RNA, 90 nt, Artificial Sequence
  Description of Artificial Sequence  RNA Aptamer RA1-HSF
gggagaauucaacugccaucuaggcaucgcgauacaaaauuaaguugaacgcgaguucuccaucuaguacuacaagcuucuggacucggu

SEQ ID NO: 16 - RNA, 47 nt, Artificial Sequence
  Description of Artificial Sequence  reduced RNA aptamer S1con
gggaccgaccagaaucaugcaagugcguaagauagucgcgggccggg

SEQ ID NO: 17 - RNA, 143 nt, Artificial Sequence
  Description of Artificial Sequence  HSF antibody-like construct
gggauucaacugccgaccgaccagaaucaugcaagugcguaagauagucgcgggucggguggcaucgcgauacaaaauuaaguugaacgcgagucccggccggccagaaucaugcaagugcguaagauagucgcgggccggcc

SEQ ID NO: 18 - DNA, 150 bp, Artificial Sequence
  Description of Artificial Sequence HSF-aptalike (DNA) construct
agcggccgtcgttgtcgaatgttctagaaaagcgaaagcttttctagaacattcgacctctgtgagacgacgcaccggtcgcaggttttgtctcacagagcgacggccgctctgtgagacgacgcaccggtcgcaggttttgtctcacag

SEQ ID NO: 19 - DNA, 130 bp, Artificial Sequence
  Description of Artificial Sequence TAQ-aptalike (DNA) construct
agcggccgtcgttgtgacaatgtacagtattggccacctctgtgagacgacgcaccggtcgcaggttttgtctcacagagcgacggccgctctgtgagacgacgcaccggtcgcaggttttgtctcacag

SEQ ID NO: 20 - RNA, 199 nt, Artificial Sequence
  Description of Artificial Sequence  aptabody B-S
gggaucgccgcggcuggucaaccaggcgaccgccgcggccacagcggugggcuggucaaccaggcgaccgcccaccagcguucccggauccgugaccgaccagaaucaugcaagugcguaagauagucgcgggucgggucauacucccggccagaaucaugcaagugcguaagauagucgcgggccgggaggcggauuc

SEQ ID NO: 21 - RNA, 169 nt, Artificial Sequence
  Description of Artificial Sequence Aptabody B-R
gggaucgccgcggcuggucaaccaggcgaccgccgcggccacagcggugggcuggucaaccaggcgaccgcccaccagcguucccggauccgugacgucaacacggucccggacguguugacguccauacuccucaacacggucccggacguguugaggaggcggauuc

SEQ ID NO: 22 - RNA, 185 nt, Artificial Sequence
  Description of Artificial Sequence  aptabody T-S
gggaucgccgccgagcccgggagcucggcggccacagcggugggagcccgggagcucccaccagcguucccggauccgugaccgaccagaaucaugcaagugcguaagauagucgcgggucgggucauacucccggccagaaucaugcaagugcguaagauagucgcgggccgggaggcggauuc

SEQ ID NO: 23 - RNA, 9 nt, Artificial Sequence
  Description of Artificial Sequence  functional element N1
uccauacuc

SEQ ID NO: 24 - RNA, 15 nt, Artificial Sequence
  Description of Artificial Sequence  functional element N2
gaggcggcgguucgc

SEQ ID NO: 25 - RNA, 12 nt, Artificial Sequence
  Description of Artificial Sequence  functional element N3
gccacagcggug

SEQ ID NO: 26 - RNA, 19 nt, Artificial Sequence
  Description of Artificial Sequence  functional element N4
caccagcguuccgccguga

SEQ ID NO: 27 - RNA, 9 nt, Artificial Sequence
  Description of Artificial Sequence  functional element N1
uccauacuc

SEQ ID NO: 28 - RNA, 23 nt, Artificial Sequence
  Description of Artificial Sequence  functional element N2
gaggcggcaguauuccgguucgc

SEQ ID NO: 29 - RNA, 12 nt, Artificial Sequence
  Description of Artificial Sequence  functional element N3
gccacagcggug

SEQ ID NO: 30 - RNA, 27 nt, Artificial Sequence
  Description of Artificial Sequence  functional element N4
caccagcguuccggaguacugccguga

SEQ ID NO: 31 - RNA, 58 nt, Artificial Sequence
  Description of Artificial Sequence  modified hammerhead ribozyme
cucgacgucuagcgaugugguuucgcuacugaugaguccgugaggacgaaacgucgag

SEQ ID NO: 32 - RNA, 43 nt, Artificial Sequence
  Description of Artificial Sequence  module N3-1
gcgauauggaaguuccggggaaacuugguucuuccuaagucgu

SEQ ID NO: 33 - RNA, 14 nt, Artificial Sequence
  Description of Artificial Sequence  module N3-2A (TAR-MAL)
gagcccgggagcuc

SEQ ID NO: 34 - RNA, 24 nt, Artificial Sequence
  Description of Artificial Sequence  module N3-2B (R-0624A54G)
ucaacacggucccggacguguuga

SEQ ID NO: 35 - RNA, 162 nt, Artificial Sequence
  Description of Artificial Sequence module N4-1 (MPMVCTE8009-8170)
uccccugugagcuagacuggacagccaaugacggguaagagagugacauuucucacuaaccuaagacaggagggccgucaaagcuacugccuaauccaaugacggguaauagugacaagaaauguaucacuccaaccuaagacaggcgcagccuccgaggga

SEQ ID NO: 36 - RNA, 21 nt, Artificial Sequence
  Description of Artificial Sequence  module N4-2 (MS2SELECT)
aaacaugaggaucacccaugu

SEQ ID NO: 37 - RNA, 213 nt, Artificial Sequence
  Description of Artificial Sequence  RNA construct without aptamer RNAHYB-VEC
uagcgaugugguuucgcuacugaugaguccgugaggacgaaacgucgaguccauacucgcggccgcnngcggccgcgaggcggcaguauuccgguucgcgcgauauggaaguuccggggaaacuugguucuuccuaagucgugccacagcggugaaacaugaggaucacccaugucccaccagcguuccggaguacugccgugacucgacguc

SEQ ID NO: 38 - RNA, 262 nt, Artificial Sequence
  Description of Artificial Sequence  RNA construct with HSF aptamer RA1-HSF (RNAHYB-RA1)
uagcgaugugguuucgcuacugaugaguccgugaggacgaaacgucgaguccauacucgcggccgcauucaacugccguucgcggcaucgcgauacaaaauuaaguugaacgcgagugcggccgcgaggcggcaguauuccgguucgcgcgauauggaaguuccggggaaacuugguucuuccuaagucgugccacagcggugaaacaugaggaucacccaugucccaccagcguuccggaguacugccgugacucgacguc

SEQ ID NO: 39 - RNA, 242 nt, Artificial Sequence
  Description of Artificial Sequence  RNA construct with NF-KB aptamer ap50 (RNAHYB-NF)
uagcgaugugguuucgcuacugaugaguccgugaggacgaaacgucgaguccauacucgcggccgcgauccugaaacuguuuuaagguuggccgaucgcggccgcgaggcggcaguauuccgguucgcgcgauauggaaguuccggggaaacuugguucuuccuaagucgugccacagcggugaaacaugaggaucacccaugucccaccagcguuccggaguacugccgugacucgacguc

SEQ ID NO: 40 - RNA, 215 nt, Artificial Sequence
  Description of Artificial Sequence  construct AptTBP12(2)MS2(1)
ugggcuaagcccacugaugagucgcugaaaugcgacgaaaccucgagucauacucgcggccgcugacgcgccgugcccgguuuggauaggcacauaagacgcgucauacuccgccgugcccgguuuggauaggcacauaagacggagggcggccgcgaggcggcaguauuaaacaugaggaucacccauguccaguacugccgugacucgacguc

SEQ ID NO: 41 - RNA, 248 nt, Artificial Sequence
  Description of Artificial Sequence  RNA construct containing one AptTBP-101 aptamer and two MS2 binding sites
ugggcuaagcccacugaugagucgcugaaaugcgacgaaaccucgagucauacucgcggccgcagaauucaacucuucggagccaagguaaacaauucaguuaguggaaugaaacuggcggccgcgaggcggcaguauuccgguucgcgcagaaacaugaggaucacccauguccugugccacagcggugaaacaugaggaucacccauguccaccagcguuccggaguacugccgugacucgacguc

SEQ ID NO: 42 - RNA, 99 nt, Artificial Sequence
  Description of Artificial Sequence  Apt TBP-101 aptamer
gggagaauucaacugccaucuaggcagccaagguaaacaauucaguuaguggaaugaaacugcccaacaccagaaguacuacaagcuucuggacucggu

SEQ ID NO: 43 - RNA, 171 nt, Artificial Sequence
  Description of Artificial Sequence Aptabody-12/S1/101
gggcauacuccggcgccgugcccgguuuggauaggcacauaagacgccggaggcggcgaccgaccagaaucaugcaagugcguaagauagucgcgggucggggccgugcccgcgagaauucaacucuucggagccaagguaaacaauucaguuaguggaaugaaacugcgc

SEQ ID NO: 44 - RNA, 198 nt, Artificial Sequence
  Description of Artificial Sequence Aptabody-B4/S1/101
gggcauacuccggcggcuaauguaggaugcugggguaguccagcccuagaauaagcgcuaguacuacaagcugccggaggcggcgaccgaccagaaucaugcaagugcguaagauagucgcgggucggggccgugcccgcgagaauucaacucuucggagccaagguaaacaauucaguuaguggaaugaaacugcgc

SEQ ID NO: 45 - RNA, 100 nt, Artificial Sequence
  Description of Artificial Sequence  Aptamer directed to TFIIB, AptB4
gggagaauucaacugccaucuaggcaaagagcuaauguaggaugcugggguaguccagcccuagaauaagcgcuaguacuacaagcuucuggagcucggu

SEQ ID NO: 46 - RNA, 37 nt, Artificial Sequence
  Description of Artificial Sequence  mini version of AptTBP-12 (m12)
ggcgccgugcccgguuuggauaggcacauaagacgcc

SEQ ID NO: 47 - RNA, 405 nt, Artificial Sequence
  Description of Artificial Sequence  precursor construct
guuuuacguuugaggccucguggcgcacaugguacgcuguggugcucgcggcugggaacgaaacucugggagcugcgauuggcagaauucacuugaggucugggcuaagcccacugaugagucgcugaaaugcgacgaaaccucgagucauacucgcggccgcugacgcgccgugcccgguuuggauaggcacauaagacgcgucauacuccgccgugcccgguuuggauaggcacauaagacggagggcggccgcgaggcggcaguauuaaacaugaggaucacccauguccaguacugccgugacucgacgucuagcgaugugguuucgcuacugaugaguccgugaggacgaaacgucgacgaauucccccauauccaacuuccaauuuaaucuuucuuuuu

SEQ ID NO: 48 - RNA, 438 nt, Artificial Sequence
  Description of Artificial Sequence  precursor construct
guuuuacguuugaggccucguggcgcacaugguacgcuguggugcucgcggcugggaacgaaacucugggagcugcgauuggcagaauucacuugaggucugggcuaagcccacugaugagucgcugaaaugcgacgaaaccucgagucauacucgcggccgcagaauucaacucuucggagccaagguaaacaauucaguuaguggaaugaaacuggcggccgcgaggcggcaguauuccgguucgcgcagaaacaugaggaucacccauguccugugccacagcggugaaacaugaggaucacccauguccaccagcguuccggaguacugccgugacucgacgucuagcgaugugguuucgcuacugaugaguccgugaggacgaaacgucgacgaauucccccauauccaacuuccaauuuaaucuuucuuuuu

SEQ ID NO: 49 - RNA, 215 nt, Artificial Sequence
  Description of Artificial Sequence  RNA construct without aptamer
uagcgaugugguuucgcuacugaugaguccgugaggacgaaacgucgaguccauacucgcggccgcuucggcggccgcgaggcggcaguauuccgguucgcgcgauauggaaguuccggggaaacuugguucuuccuaagucgugccacagcggugaaacaugaggaucacccaugucccaccagcguuccggaguacugccgugacucgacguc

SEQ ID NO: 50 - RNA, 100 nt, Artificial Sequence
  Description of Artificial Sequence  two AptTBP-12 aptamers
gggagaauucaacugccaucuaggcgccgugcccgguuuggauaggcacauaagacgccgacaaagaaaccaaccaguacuacaagcuucuggacucggu